(19) United States
(12) Patent Application Publication
(10) Pub. No.: US 2021/0165003 A1
(43) Pub. Date: Jun. 3, 2021

(54) ASSESSMENT OF THE RISK OF COMPLICATION IN A PATIENT SUSPECTED OF HAVING AN INFECTION, HAVING A SOFA SCORE LOWER THAN TWO

(71) Applicant: BIOMÉRIEUX, Marcy L'Etoile (FR)

(72) Inventors: Marie-Angélique CAZALIS, Vaulx en Velin (FR); Karine KAISER, Lezoux (FR); Alexandre PACHOT, Sulignat (FR)

(73) Assignee: BIOMÉRIEUX, Marcy L'Etoile (FR)

(21) Appl. No.: 17/048,131

(22) PCT Filed: Apr. 15, 2019
(86) PCT No.: PCT/FR2019/050884

§ 371 Date: Oct. 16, 2020

(30) Foreign Application Priority Data
Apr. 16, 2018 (FR) 1853295

(51) Int. Cl. G01N 33/74

(52) U.S. Cl. CPC G01N 33/74; G01N 2800/56; G01N 2333/71; G01N 2333/9723; G01N 2333/9726; G01N 2800/26

(57) ABSTRACT

A method for the in vitro or ex vivo assessment of the risk of complications in a patient suspected of having an infection, having a SOFA score of less than two, including measuring the level of expression, in a biological sample obtained from said patient, of at least one expression product of the VEGFR2 gene.

The present invention relates to the field of medicine in general, and in particular to the field of in vitro prognostics. More specifically, it relates to the assessment of the risk of complications in a patient suspected of having an infection, having a SOFA score of less than two.

One of the principal risks of complications for a patient who has contracted an infection is the development of a septic syndrome. Sepsis and septic shock are medical emergencies which affect almost 18 million people across the globe. In Europe, the annual mortality rate is of the order of 30% to 40%. These patients need care in specialized units with hospital stays which are often of more than a month and generate very high associated costs.

Sepsis is a complex syndrome which passes through several clinical phases. It is associated with a high mortality rate. Early recognition of patients at risk of an unfavorable outcome is one of the determining elements of prognostics.

Until 2016, the identification of sepsis was based on a classification from 1991 and updated in 2001. It distinguished four phases considered to be phases of progressive aggravation of the infection and of the inflammatory response to it: infection, sepsis, severe sepsis and septic shock. Thus, before 2016, sepsis was defined as the presence of an infection associated with signs of systemic inflammation of the organism (Systemic Inflammatory Response Syndrome, SIRS). A group of experts proposed criteria for defining the following clinical syndromes (International Sepsis Definitions Conference, Crit. Care Med. 31; 2003):
SIRS is a systemic inflammatory response triggered by a variety of causes, infectious or otherwise. SIRS conditions triggered by non-infectious causes that may be cited include trauma, burns, pancreatitis, acute respiratory syndromes. An inflammatory systemic response manifest itself with by at least two of the following signs: a) a temperature of less than 38° C. or less than 36° C.; b) heart rate of more than 90 beats per minute; c) respiratory rate of more than 20 breaths per minute; d) number of leukocytes more than 12000/mm³ or less than 4000/mm³,
sepsis is a systemic inflammatory response syndrome related to an infection,
severe sepsis is sepsis associated with arterial hypotension and/or hypoperfusion and/or dysfunction of at least one organ,
septic shock is severe sepsis associate with persistent hypotension and may be qualified by:
   the presence of an identified infectious site,
   persistento hypotension despite adequate fluid replacement and treatment with vasopressors.

These definitions suffers from a number of limitations, in particular an excessive focus on inflammation, as well as an erroneous model of sepsis as a continuum moving from severe sepsis then to septic shock. In addition, the specificity and the sensitivity of the criteria for systemic inflammatory response syndrome (SIRS) were insufficient. An international group of experts (SEPSIS-3), seeking to simplify and improve the classification of acute septic conditions emphasized that many definitions and a lot of different terminologies were used for septicemia, septic shock and for the dysfunction of certain organs, leading to anomalies in the reported incidence and observed mortality.

Thus, at the beginning of 2016, the SEPSIS-3 international consensus conference redefined sepsis as being a life-threatening organ dysfunction and caused by an inappropriate response by the host to an infection. Since then, only sepsis and septic shock have been distinguished. These new definitions, which emphasize the notion of life-threatening organ dysfunction, uses a score termed the SOFA score (sequential organ failure assessment score) and its simplified version, qSOFA (quick SOFA), defined below, in order to determine the association between mortality and organ failure. From a clinical viewpoint, organ dysfunction is characterized either by an infection associated with a SOFA score of 2 or higher, or by an increase in the SOFA score of at least 2 points.

The SOFA score (sequential organ failure assessment score) is used to monitor the status of a person during their hospital stay in order to determine the extent of organ failures (Vincent J L et al., 1996). The score is based on six different scores, one for each of the following systems: respiratory, cardiovascular, hepatic, coagulation, renal and neurological. The mode of calculation for each system is indicated in Table 1 below:

TABLE 1
RESPIRATORY SYSTEM

| PaO₂/FiO₂ (mmHg) | SOFA score |
|---|---|
| ≥400 | 0 |
| <400 | 1 |
| <300 | 2 |
| <200 and mechanical ventilation | 3 |
| <100 and mechanical ventilation | 4 |

NEUROLOGICAL SYSTEM

| Glasgow scale | SOFA score |
|---|---|
| 15 | 0 |
| 13-14 | 1 |
| 10-12 | 2 |
| 6-9 | 3 |
| <6 | 4 |

CARDIOVASCULAR SYSTEM

| Arterial pressure and vasopressor treatment | SOFA score |
|---|---|
| Mean arterial pressure (MAP) ≥ 70 mm/Hg | 0 |
| Mean arterial pressure (MAP) < 70 mm/Hg | 1 |
| Dopamine ≤ 5 μg/kg/min or dobutamine | 2 |
| Dopamine > 5 μg/kg/min or epinephrine ≤ | 3 |
| 0.1 μg/kg/min or norepinephrine ≤ | |
| 0.1 μg/kg/min | |
| dopamine > 15 μg/kg/min or epinephrine > | 4 |
| 0.1 μg/kg/min or norepinephrine > | |
| 0.1 μg/kg/min | |

HEPATIC SYSTEM

| | |
|---|---|
| Bilirubin (mg/dl) [μmol/L] | SOFA score |

| | | |
|---|---|---|
| <1.2 | [<20] | 0 |
| 1.2-1.9 | [20-32] | 1 |
| 2.0-5.9 | [33-101] | 2 |
| 6.0-11.9 | [102-204] | 3 |
| >12.0 | [>204] | 4 |

COAGULATION

| Platelets × 10³/μl | SOFA score |
|---|---|
| ≥150 | 0 |
| <150 | 1 |
| <100 | 2 |
| <50 | 3 |
| <20 | 4 |

RENAL SYSTEM

| | |
|---|---|
| Creatinine (mg/dl) [μmol/L] (or urine output) | SOFA score |

| | | |
|---|---|---|
| <1.2 | [<110] | 0 |
| 1.2-1.9 | [110-170] | 1 |
| 2.0-3.4 | [171-299] | 2 |
| 3.5-4.9 | [300-440] (or <500 ml/d) | 3 |
| >5.0 | [>440] (or <200 ml/d) | 4 |

The qSOFA score (or quickSOFA) was introduced in February 2016 in the form of a simplified version of the SOFA score as an initial means for identifying patients at high risk of a poor outcome after an infection (Eamon P. Raith, et al., 2017). The qSOFA considerably simplifies the SOFA score by only including 3 clinical criteria. The qSOFA can therefore be calculated easily and rapidly and can be carried out repeatedly on patients. Table 2 below gives the method for calculating qSOFA.

TABLE 2

| Assessment | qSOFA score |
|---|---|
| Systolic arterial pressure ≤ 100 mmHg. | 1 |
| Respiratory rate ≥ 22/min | 1 |
| Higher function problems (confusion, Glasgow < 15) | 1 |

The qSOFA score can thus vary from 0 to 3 points.

The presence of 2 or more qSOFA points close to the onset of infection is associated with a greater risk of death or a prolonged stay in an intensive care unit.

Any patient presenting with sepsis can develop rapidly towards much more severe and fatal forms such as septic shock. The challenge in patient care is therefore to intervene as rapidly as possible, and in fact even before the patient presents with sepsis, in order to prevent tissue hypoxia from occurring and shock, which could lead to an irretrievable solution.

In current practice, early recognition among patients who are suspected of only having an infection without a risk of aggravation from those who are at risk of complications is not always simple. There are currently no tools that are available. In this context, a triage system which could identify patients presenting risks of major clinical complications, in particular complications leading to sepsis, is a recurring medical demand.

The identification among patients suspected of having a non-serious infection (having a SOFA score of less than 2) who are the most at risk of complications and as early as possible and in the absence of sepsis would enable a strategy for limiting the risk to them of complications to be put in place as early as possible. As an example, a prophylactic antibiotic treatment could be administered (Puisieux F et al., 1993; Jensen J U et al., 2011), or in fact a preventative treatment (K. Asehnoune et al., 2014), or in fact targeted immunotherapy (Chahin A et al., 2015; Ali Y M et al., 2014) , or more simply, pathogen access pathways could be limited (i.e. removal of catheters as soon as possible, etc.). These measures would enable better care of the patient, leading to:
a reduction in the time spent in intensive care and in hospital, which would reduce the associated costs (Lambert M L S C et al., 2011);
a decrease in septic complications; and
a decrease in mortality rate.

There is therefore an urgent need, which has not been resolved for many years, to find good tools, in particular including good markers, which are capable of assessing the risk of complications in patients suspected of having an infection but without signs of severity and characterized by a SOFA score of less than two, and therefore enable them to be stratified as a function of their risk of complications.

Various markers have already been described in order to assist in the diagnosis of sepsis and sometimes in the context of assessing the risk of complications in patients who already present with sepsis. Examples of markers of this type include procalcitonin (PCT) or C-Reactive Protein (CRP).

In addition to procalcitonin, application WO 2013/152047 discloses more than a hundred biomarkers for their use in the diagnosis of sepsis, or in fact of septic shock, but also in the prognosis of the development of sepsis already developing into septic shock, in accordance with the old definition. These biomarkers include the vascular endothelial growth factor receptor 2 (VEGFR2). However, the patient cohorts described in the examples concerning this marker for the prognosis of development were composed of patients who had been admitted to an intensive care unit for at least 48 hours, specialized hospital units dedicated to the care of critically ill patients or particularly seriously ill patients necessitating permanent monitoring. These patients had a SOFA score of three or more. They were patients for whom the clinical condition was particularly serious, which does not in any way correspond to patients suspected of having an infection, having a SOFA score of less than two. The patients simply suspected of having an infection and having a SOFA score of less than two would prima facie not be considered by health professionals to be serious. The patients described in that patent application resembling patients suspected of having an infection and having a SOFA score of less than two are those of Examples 4 and 11. However, for those patients, VEGFR2 was never described as being associated with a risk of aggravation.

In the same way, Wada and his team (Wada T et al., 2013) describe soluble receptors with angiogenic factors, such as sVEGFR2 or in fact angiopoietin 2, a growth factor involved in angiogenesis, as being predictive of the development of an acute respiratory distress syndrome (ARDS) in seriously ill patients. Here again, the patients included in the cohort for that publication were seriously sick patients, already hospitalized, all breathing under mechanical ventilation and having either sepsis or severe trauma, or being in intensive care following a cardiac arrest. Those patients had very high SOFA scores ranging from four to almost ten. Here again, patients receiving respiratory assistance are not patients simply suspected of having an infection, having a SOFA score of less than two, not considered to be serious.

VEGFR2 is one of the vascular endothelial growth factor receptors (VEGF). This latter is the principal factor involved in neoangiogenesis. The VEGF family in mammals is composed of four glycoproteins referred to as A to D and placental growth factor (PIGF) (Koch and Claesson-Welsh, 2012). Each of them is expressed in different isoforms due to alternative splicing and proteolitic cleavage. These VEGFs bind and activate three types of tyrosine kinase receptors, VEGFR2, but also VEGFR 1 and 3 respectively known as KDR, Flt1 and VEGFR3. VEGFs also bind the neuropilins NP1 and NP2, which act as co-factors for the VEGFRs.

The VEGFR2 gene, also known as KDR, (this gene will only be referred to below as VEGFR2) codes for the vascular endothelium growth factor type 2 receptor. It is a tyrosine kinase protein that acts as a cellular surface receptor for VEGFA, VEGFC and VEGFD. It plays a vital role in the regulation of angiogenesis, vascular development, vascular permeability and embryonic hematopoiesis. This receptor promotes proliferation, survival, migration and differentiation of endothelial cells, but also the reorganization of the actin cytoskeleton. This receptor is constituted by an extracellular portion constituted by 7 immunoglobulin type domains, a transmembrane region and an intracellular portion containing a tyrosine kinase domain (Shibuya, 2011).

Thus, the pharmaceutical industry has become very interested in these receptors in the context of cancer treatments. Molecules intended to block the activation of VEGFR have been developed, enabling angiogenesis and, as a consequence, tumor proliferation, to be controlled.

Although vascular endothelium growth factor type 2 receptor or VEGFR2 has many applications in the medical field, it has never before been described as a marker for complications in a patient simply suspected of having an infection and having a SOFA score of less than two, in other words in a patient not considered to be seriously sick. Counter to expectations, the Applicant has shown that measuring the level of expression of the VEGFR2 gene can be used to assess the risk of complications in a patient suspected of having an infection at a very early stage even before the appearance of signs of severity.

For this reason, the invention therefore presents a major advance in the field of stratification of patients. The method in accordance with the invention has the advantage of easily being able to assess the risk of complications in a patient suspected of having an infection and presenting no clinical signs of severity (SOFA score of less than two), by providing a marker that is directly measurable, in particular by automated analytical instruments, and wherein the measurement can be made in a local laboratory upon arrival at an emergency center, by the treating physician or even at the patient's bedside, and thus carry out triage linked to the necessity of caring for patients who are the most at risk from among all of these patients suspected of having an infection.

Thus, in a first aspect, the invention provides a method for the in vitro or ex vivo assessment of the risk of complications in a patient suspected of having an infection, having a SOFA score of less than two, comprising measuring the level of expression, in a biological sample obtained from said patient, of at least one expression product of the VEGFR2 gene.

The invention also concerns a method for stratification and treatment of a patient suspected of having an infection, having a SOFA score of less than two, characterized in that it comprises the steps consisting of:
identifying the patients presenting a risk of complications by carrying out the method in accordance with the invention for the assessment, in vitro or ex vivo, of the risk of complications in that patient, and
adapting the health care management of said patient identified in the preceding step in order to reduce the risk of complications.

In another aspect, the invention concerns a kit for measuring, in vitro or ex vivo, the level of expression of at least one expression product of the VEGFR2 gene and of at least one expression product of the uPAR gene in a patient suspected of having an infection, having a SOFA score of less than two, comprising at least one specific binding partner for said at least one expression product of the VEGFR2 gene and at least one specific binding partner for said at least one expression product of the uPAR gene.

Finally, in a final aspect, the present invention provides a kit for measuring, in vitro or ex vivo, the level of expression of at least one expression product of the VEGFR2 gene and at least one expression product of the uPAR gene, in a biological sample, comprising:
specific tools or reagents enabling the quantities of said at least one expression product of the VEGFR2 gene and of said at least one expression product of the uPAR gene in said biological sample to be measured, and
a control calibrated to contain the quantities of said at least one expression product of the VEGFR2 gene which correspond to known quantities of said at least one expression product of the VEGFR2 gene, and
a control calibrated to contain the quantities of said at least one expression product of the uPAR gene which correspond to known quantities of said at least one expression product of the uPAR gene.

Before going further in the description of the invention, the following definitions shall be provided in order to facilitate comprehension.

In the context of the invention, “patients” are patients suspected of having an infection and having a SOFA score of less than two. In other words, they are patients not presenting clinical signs of severity.

The term “patient suspected of having an infection” means a patient having a proven infection or being suspected of having an infection by a health professional on clinical or paraclinical signs.

A “health professional” is any person using medical skill and judgement, providing a service linked to maintaining or improving the health of patients, or treating individuals who are injured, sick, suffering from a disability or infirmity and providing care.

By way of example, we can cite the following health professionals: the physician, nurse or in fact the midwife.

By way of non-exhaustive example of clinical signs, we may cite the following:
for a pulmonary site of infection: thoracic pain, dyspnea, anything directing the health professional towards a pulmonary site of infection;
for a cutaneous site of infection: erythema or other inflammatory cutaneous lesion;
for a urinary tract infection (lower or upper): micturition burning, pollakiuria or dysuria, pelvic pain, emission of odor from the urinary meatus, unilateral lumbar pain;
for a neuromeningeal site of infection: headaches, photophobia, myalgia and arthralgia, difficulty concentrating;
a digestive site of infection: abdominal pains, bowel problems;
temperature higher than 38° C. or lower than 36° C.;
heart rate more than 90 beats per minute;
respiratory rate more than 20 breaths per minute;
number of leukocytes more than 12000/mm³ or less than 4000/mm³.

Non-exhaustive examples of paraclinical signs that we can cite are:
measuring procalcitonin;
lactatemia;
measuring C-reactive protein;
hepatic balance;
measuring ALAT/SGPT (Alanine-Aminotransferase/Serum Glutamopyruvate Transferase) and ASAT/SGOT (Aspartate-Aminotransferase/Serum Glutamooxaloacetate Transferase);
renal balance (urea and creatinine);
blood electrolytes;
cytobacteriological urine examination;
arterial gasometry;
coagulation.

With the aid of clinical or paraclinical signs, the health professional will determine whether the patient is suspected of having an infection or not. The patient suspected of having an infection will present one or more clinical and/or paraclinical signs, for example those exemplified above.

In the context of the invention, the infection may be caused by contamination of the patient with any infectious agent such as a virus, a bacterium, a parasite, a fungus or indeed a protozoan.

Examples of infectious agents that we can cite are viruses such as the HIV, SIV, FIV, VHC, VHB, VHA, VHE virus, the VZV, CMV, EBV, VHS1, VHS2 virus, bacteria such as Mycobacterium tuberculosis, Mycobacterium bovis, Mycobacterium leprae, Borrelia burgdorferi stricto sensu, Borrelia afzelii, Borrelia garinii, Borrelia spielmanii, Clostridium difficile, Clostridium botulinum, Salmonellas, Klebsiella, Legionella, Proteus, Klebsiella, Escherichia coli, Shigella, Pseudomonas aeruginosa, Staphylococcus aureus, Treponema pallidum, yeasts such as Candida albicans, fungi such as Aspergillus fumigatus, Mucorales, etc. and protozoa such as Leishmania, Trichomonas vaginalis, Plasmodium, etc.

Similarly, when the patient is suspected of having an infection, it can affect any tissue or organ. Examples of infections that may be cited are those:
of the urinary tract and genitalia;
of the abdominal organs;
from wounds and soft tissues;
of the skin;
via catheter;
of the central nervous system;
of the heart valves;
of the digestive system in its entirety or in part (stomach, duodenum, small intestine, colon, rectum and anus),
the bladder,
the intestines (duodenum, small intestine, colon, rectum and anus);
of the ear, nose and throat.

In accordance with a particular embodiment of the invention, the infection is bacterial in origin.

In accordance with the present invention, the patient is characterized by their SOFA score which is strictly less than 2, in other words a patient having a SOFA score of either 0 or of 1. In other words, in accordance with the new definition from 2016 (SEPSIS-3), they are patients not presenting with sepsis.

The patients in accordance with the invention are therefore patients not presenting a serious clinical condition. As an example, the patients are not in intensive care and/or are not being mechanically ventilated.

The patients in accordance with the invention, who are not in a serious clinical condition, will be brought into contact with health professionals who will suspect an infection, for example in the following cases upon arrival in the emergency center, by the treating physician themselves, or in fact in hospital at their bedside, which constitutes another particular embodiment.

The term “assessment of the risk of complications” means identifying, from among patients suspected of having an infection and having a SOFA score of less than two, those whose condition will deteriorate over the hours following the measurement of the level of expression of the marker and those whose condition will not deteriorate over the following hours and who, as a consequence, will not require care by a specialized hospital unit.

In accordance with the present invention, the complications are characterized by at least one of the following events:
an increase of at least one point in the SOFA score,
the appearance of failure of at least one organ,
the need to go into intensive care, and
death.

In the context of the invention, the term “organ failure” means abnormal function of an organ, which results in a clinical or biological anomaly in the organ (see below for calculation of the SOFA score).

Examples of organ failure that may be cited are respiratory, cardiovascular, renal, neurological, hepatic failure, or in fact hematological failures.

With respect to clinical or biological anomalies, the definitions according to Fagon regarding organ failure may be cited by way of example (Fagon et al., 1993):
respiratory failure (at least one of the following criteria):
   PaCO₂<60 mmHg with FIO2=0.21
   artificial ventilation
cardiovascular failure (at least one of the following criteria in the absence of hypovolemia):
   systolic arterial pressure<90 mmHg with signs of peripheral hypoperfusion
   use of inotropic drugs or vasopressors in order to maintain a systolic arterial pressure>90 mmHg
cardiovascular failure (at least one of the following criteria in the absence of hypovolemia):
   systolic arterial pressure<90 mmHg with signs of peripheral hypoperfusion
   use of inotropic drugs or vasopressors in order to maintain a systolic arterial pressure>90 mmHg
renal failure (at least one of the following criteria in the absence of chronic renal insufficiency):
   Creatininemia>300 μmol/L
   Diuresis<500 mL/24 h or <180 mL/8 h
   Need for renal dialysis
neurological failure (at least one of the following criteria):
   Glasgow score≤6 (in the absence of sedation)
   Sudden onset of delirium
hepatic failure (at least one of the following criteria):
   bilirubin>100 μmol/L
   alkaline phosphate>x3
hematological failure (at least one of the following criteria):
   hematocrit≤20%
   leukocytosis<2 000/mm³
   platelets<40 000/mm³

In the context of the present invention, a patient needs to go into intensive care when their condition necessitates continuous monitoring of vital functions and, if appropriate, recourse to support methods (transfusion of blood derivatives, vascular fluid replacement, mechanical ventilation, catecholamines, hemodialysis, extracorporeal circulation, etc.). The final objective of intensive care is to restore homeostasis.

The presence of a risk of complications corresponds to the risk that the complication will develop in the 5 days, in particular in the 4 days, especially in the 3 days, for example, which follow removal of a sample and when several samples have been taken in the 5 days, in particular in the 4 days, especially in the 3 days which follow taking the first sample.

The method in accordance with the invention comprises measuring the level of expression, in a biological sample obtained from said patient, of at least one expression product of the VEGFR2 gene.

In general, the term “sample” refers to a portion or to a quantity, more particularly a small portion or a small quantity, removed from one or more entities for the purpose of analysis. This sample may optionally have undergone a previous treatment, for example involving steps for mixing and dilution.

The sample in the context of the method of the invention is a biological sample from a patient suspected of having an infection, having a SOFA score of less than two for whom the risk of complications in the hours which follow measuring the level of expression of the marker or markers is to be assessed. In particular, a biological sample of this type is selected from those which are susceptible of containing an expression product of VEGFR2 or any other marker described below.

The biological sample in accordance with the present invention may vary in nature. In particular, this sample is a biological fluid, for example selected from whole blood (such as that collected from a vein, i.e. containing white and red cells, platelets and plasma), serum, plasma, bronchoalveolar lavage fluid, cerebrospinal fluid, also known as spinal fluid, and urine. Preferably, the biological sample obtained from the patient is a sample of whole blood, plasma, serum or any derivative.

In accordance with the present invention, the measurement of the level of expression of the VEGFR2 gene (SEQ ID No 1) consists of quantifying at least one expression product of this gene.

The “expression product” in the context of the invention is any biological molecule obtained from expression of the gene for VEGFR2. Examples which may be mentioned are an RNA transcript, a protein or a polypeptide.

In accordance with one embodiment of the invention, the expression product of the gene is an RNA transcript. The term “transcript” means RNA, and in particular messenger RNA, obtained from transcription of the VEGFR2 gene. More precisely, the transcripts are the RNAs produced by gene splicing. Thus, in this embodiment, the measurement of the level of expression of one or more RNA transcripts of the VEGFR2 gene may be carried out.

The VEGFR2 gene has three currently known transcripts, recorded in the Ensembl (GRCh38.p10) database and identified in Table 3 below.

TABLE 3

| Name of | Identification | Theoretical | |
|---|---|---|---|
| transcript | number | protein size | Sequences |
| KDR-201 | ENST00000263923.4 | 1356 aa | SEQ ID No2 |
| KDR-202 | ENST00000509309.1 | No protein | — |
| KDR-203 | ENST00000512566.1 | No protein | — |

In accordance with one embodiment, the expression of one, two or three transcripts selected from the transcripts KDR-201 (SEQ ID No 2), KDR-202 and KDR-203 and their variants is carried out. The term “variant” means an RNA with a sequence having at least 99% identity with one of said sequences for the transcripts KDR-201, KDR-202, KDR-203. The percentage identity is determined by means of sequence alignment software, such as CLUSTALW (Thompson et al., 1994). In particular, a variant will correspond to a polymorphism of the sequence for the VEGFR2 gene. The preferred transcript of the VEGFR2 gene is the transcript KDR-201 or one of its variants having at least 99% identity with the sequence of said transcript, the expression of which will be determined alone or in association with that of other variants. Preferably, only the expression of transcript KDR-201 or of one of its variants having at least 99% identity with the sequence of said transcript will be determined.

Any method for measuring the level of expression of the RNA transcript that is well known to the person skilled in the art may be used to carry out the invention.

As a general rule, measuring the level of expression of an RNA transcript of a target gene may comprise a preliminary step for extraction of total RNA from a biological sample. This step is followed by a step for reverse transcription of these various RNAs in order to obtain their complementary DNA (cDNA). Next, the specific cDNAs of the target gene are amplified then quantified.

Extraction is carried out using any of the protocols for the extraction and purification of nucleic acids which are well known to the person skilled in the art. By way of indication, nucleic acids may be extracted by lysis of cells present in the biological sample followed by purification, or in fact by extraction with phenol, chloroform and alcohol. These steps are well known to the person skilled in the art and have been described by Sambrook J and Russell DW (2017), for example.

These steps can be used to extract total RNAs from the biological sample comprising ribosomal RNA, transfer RNA and messenger RNA.

Next, a reverse transcription reaction is carried out with the aid of a reverse transcriptase enzyme which can be used to obtain a complementary DNA fragment (cDNA) from an RNA fragment. Carrying out a step of this type is well known to the person skilled in the art (Bustin S A Journal of Molecular Endocrinology, 2002, 29: 23-39; Giulietti A Methods, 2001, 25: 386-401). When, in particular, only complementary DNA is to be obtained from messenger RNA, this enzymatic step is carried out in the presence of nucleotide fragments comprising only thymine bases (polyT), which hybridize to the polyA of the various mRNAs by complementarity in order to form a polyT-polyA complex which then acts as a starting point in the reverse transcription reaction carried out by the reverse transcriptase enzyme. This produces different complementary DNAs of the different messenger RNAs initially present in the biological sample.

Next, an enzymatic amplification reaction is carried out with the aim of specifically amplifying the specific cDNAs for the target gene. An enzymatic amplification reaction is a process generating multiple copies of a nucleotide fragment by the action of at least one enzyme. Amplification reactions of this type are well known to the person skilled in the art and the following techniques in particular may be cited:
PCR (Polymerase Chain Reaction), as described in patents U.S. Pat. Nos. 4,683,195, 4,683,202 and 4,800,159;
LCR (Ligase Chain Reaction), disclosed, for example, in patent application EP 0 201 184;
RCR (Repair Chain Reaction), described in patent application WO 90/01069;
3SR (Self Sustained Sequence Replication) with patent application WO 90/06995;
NASBA (Nucleic Acid Sequence-Based Amplification) with patent application WO 91/02818;
TMA (Transcription Mediated Amplification) in patent U.S. Pat. No. 5,399,491, and
LAMP (Loop mediated isothermal amplification) with patent U.S. Pat. No. 6,410,278.

The term “amplicons” is used to designate polynucleotides generated by an enzyme amplification technique. Preferably, when the enzymatic amplification is a PCR, the specific reagent comprises at least two specific amplification primers in order to amplify a particular region of the complementary DNA of the mRNA obtained from the target gene. When the enzymatic amplification is a PCR carried out after a reverse transcription reaction, this is known as RT-PCR.

Following this amplification step, the level of expression of the target gene is determined by hybridization with the aid of at least one specific hybridization probe for the expression product of this target gene.

The term “hybridization probe” means a nucleotide fragment comprising 5 to 100 nucleotide motifs, in particular 6 to 35 nucleotide motifs, having a hybridization specificity under predetermined conditions in order to form a hybridization complex with a target nucleotide fragment. In the present invention, the target nucleotide fragment may be a nucleotide sequence included in a messenger RNA or a nucleotide sequence included in a complementary DNA obtained by reverse transcription of said messenger RNA.

Hybridization techniques are well known to the person skilled in the art; the Northern Blot technique in particular may be cited.

The quantification of mRNAs, expression products of the target gene, involves a step for detecting the hybridization reaction between the hybridization probe and the target nucleotide fragment.

The term “detection” means either direct detection using a physical method, or a detection method with the aid of a marker. Many detection methods exist for the detection of nucleic acids (Keller G. H., 1993; Kricka, 1999). In the majority of cases, this detection step does not give rise to a result during the method in accordance with the invention which is visible by the individual carrying out the invention. In accordance with one embodiment, the quantity of said at least one transcript of the VEGFR2 gene will be determined by at least one of the following characteristics, taken alone or in combination:
by quantitative enzymatic amplification, preferably by real time PCR (quantitative RT-PCR or QPCR);
with the aid of at least one hybridization probe and/or
with the aid of at least one amplification primer.

The determination of the quantity of several transcripts may be carried out sequentially or simultaneously, using conventional methods that are known to the person skilled in the art, as described above.

In another embodiment of the invention, the expression product that is measured is a protein and/or a polypeptide which is the product of translation of at least one of the transcripts described above or a transcript that has not yet been described. Thus, in this embodiment, the measurement of the level of expression of one or more proteins and/or polypeptides may be carried out.

Currently, three isoforms of VEGFR2 obtained from the KDR-201 transcript are known, including isoform 1 (SEQ ID No 3), No UniProt P35968-1, which is the transmembrane form, and isoforms 2 (SEQ ID No 4), No UniProt P35968-2, and 3 (SEQ ID No 5), No UniProt P35968-3, which are the secreted plasma forms, or soluble forms, of the receptor. These latter are designated as sVEGFR2.

All of the isoforms of VEGFR2 may be used, alone or in combination, as marker(s) in order to assess the risk of complications in a patient suspected of having an infection, having a SOFA score of less than two.

The determination of the quantity of one or more isoforms in a biological sample may be carried out in accordance with techniques which are well known to the person skilled in the art for determining the quantity, or dose, of one or more analytes in a biological sample. Examples that may be cited are assays by immunoassay, such as ELISA (Enzyme Linked Immuno Sorbent Assay), ELFA (Enzyme Linked Fluorescent Assay) and RIA (Radio Immuno Assay), and assays using mass spectroscopy, constituting one embodiment of the invention.

Assay by immunoassay is a method that is well known to the person skilled in the art and widely used in the field of the analysis of biological samples. It can be used to quantify analytes in samples, in particular in the form of proteins (antigens/antibodies), peptides and haptenes such as steroids, for example, or vitamins, involving immunological reactions between the analyte to be detected, in the present case one of the isoforms of VEGFR2, and one or more binding partner(s) for this analyte. These immunoassay methods are based on measurements for quantifying the signals emitted during the analysis of the biological sample. The quantity of signals detected is generally proportional to the quantity, or dose, of the analyte to be measured (for example during a sandwich assay) or inversely proportional to the quantity, or dose, of analyte to be measured (for example competitive assay). Clearly, the term “immuno” in “immunoassay”, for example, should not be considered in the present application to strictly indicate that the binding partner is an immunological partner such as an antibody. In fact, the person skilled in the art also uses this term broadly when the binding partner, also termed the ligand, is not an immunological partner but is, for example, a receptor for the analyte which is to be quantified. Thus, it is known to use the term ELISA (“Enzyme-Linked Immunosorbent Assay”) for assays which use non-immunological binding partners, more widely known by the English term “Ligand Binding Assay”, while the term “immuno” is included in the acronym ELISA. For the purposes of clarity, the Applicants will use the term “immuno” throughout the application for any assay using a binding partner even when it is not an immunological partner.

An example of a binding partner for isoforms of VEGFR2 that may be cited are antibodies, antibody fractions, nanofitins, aptamers (Ochsner U. A. et al., 2014) or any other molecule that is known to have an interaction with the VEGFR2 to be investigated, such as lipopolysaccharides (Bucki R. et al., 2005).

Examples of antibody binding partners are either polyclonal antibodies or monoclonal antibodies, the production of which is well known to the person skilled in the art. Antibodies of this type for the isoforms of VGEFR2 are commercially available such as, for example, the following polyclonal antibodies: Human VEGF R2/KDR/Flk-1 Biotinylated Antibody ref: BAF357 Bio-Techne® and the following monoclonal antibodies: Human VEGF R2/KDR/Flk-1 Antibody ref: MAB3573 Bio-Techne®.

Examples of antibody fragments that may be cited are the fragments Fab, Fab′, F(ab′)2 as well as scFv (Single chain variable fragment), dsFv (Double-stranded variable fragment) chains. These functional fragments may in particular be obtained by genetic engineering.

Immunoassay for determining the quantity of the isoform or isoforms of VEFGR2 preferably employs two binding partners of the isoforms of VEGFR2. One of the two partners may be coupled to a marker in order to form a conjugate or tracer. The other binding partner may be captured on a solid support. This is then known as a capture partner for the latter and detection partner for the former.

As indicated above, the measured signal emitted during the immunoassay is then proportional to the quantity, or dose, of the VEGFR2 isoform in the biological sample.

In order to correlate the signal obtained with the quantity or with the concentration in the biological sample, a mathematical model pre-established from a standard curve is utilized. This standard curve will have been obtained earlier in known manner. Briefly, obtaining a standard curve consists of measuring the signal generated by increasing quantities or concentrations of the isoform VEGFR2 which are known, of plotting the curve giving the signal as a function of the quantity or concentration, and of finding a mathematical model which represents this relationship as closely as possible. The mathematical model will be used to determine the quantities or concentrations of unknown VEGFR2 isoforms contained in the biological sample to be tested, by extrapolation.

The term “marker used to form the conjugate” in particular means any molecule containing a group which reacts with a group of the binding partner directly without chemical modification or after chemical modification in order to include such a group, the molecule being capable of directly or indirectly generating a detectable signal. A non-limiting list of these direct detection markers consists of:
enzymes that produce a signal which is detectable by colorimetry, fluorescence, luminescence, such as horseradish peroxidase, alkaline phosphatase, β-galactosidase, glucose-6-phosphate dehydrogenase;
chromophores such as fluorescent, luminescent, dye compounds;
radioactive molecules such as 32P, 35S or 125I;
fluorescent molecules such as Alexa or phycocyanins; and
electrochemiluminescent salts such as organometallic derivatives based on acridinium or ruthenium.

Indirect detection systems may also be used such as, for example, ligands that are capable of reacting with an anti-ligand. The ligand then corresponds to the marker in order to constitute the conjugate with the binding partner.

Ligand/anti-ligand pairs are well known to the person skilled in the art; this is the case, for example, with the following pairs: biotin/streptavidin, haptene/antibody, antigen/antibody, peptide/antibody, sugar/lectin, polynucleotide/polynucleotide complement.

The anti-ligand can then be detected directly by the direct detection markers described above or can themselves be detected by another ligand/anti-ligand pair, and so on.

Under certain conditions, these indirect detection systems can result in signal amplification. This signal amplification technique is well known to the person skilled in the art; reference should be made to the Applicants prior patent applications FR 2 781 802 or WO 95/08000.

Depending on the type of labeling used, the person skilled in the art will add reagents enabling the labeling to be visualized or enabling emission of a signal that can be detected using any type of appropriate measuring apparatus such as a spectrophotometer, a spectrofluorometer, a densitometer or in fact a high definition camera, for example.

The immunoassay may also include other steps that are known to the person skilled in the art, such as washing and incubation steps.

The immunoassay may be a one-step or two-step assay, as is well known to the person skilled in the art. briefly, a one-step immunoassay comprises bringing the sample to be tested into the simultaneous presence of two binding partners, while a two-step immunoassay comprises firstly bringing the test sample into the presence of the first binding partner, then the analyte—first binding partner complex formed in this manner is brought into the presence of the second binding partner.

Mass spectrometry may be used instead of the techniques described above, which are largely immunoassays. It is carried out in a mass spectrometer. This is a powerful tool, which is being used more and more for the analysis and quantification of different types of molecules in biological samples. In general, any type of molecule that can be ionized may be quantified as a function of its molecular mass with the aid of a mass spectrometer. Depending on the nature of the molecule to be quantified, of protein or metabolic origin, certain mass spectrometry technologies may be more suitable. Nevertheless, irrespective of the mass spectrometry method used for quantification, this latter comprises a step for ionization of the target molecule into what are known as molecular ions, and a step for separation of the molecular ions obtained as a function of their mass. A mass spectrometer measures the mass to charge (m/z) ratio of ionized molecules which is correlated to the target molecule to be analyzed.

This is a method that is widely known to the person skilled in the art and described in patent application WO 2016/181066 filed by the Applicant.

In the context of the invention, when a plurality of expression products are measured, they may be of the same nature, but also of different natures. In other words, they may be molecular in nature (RNA transcript, mRNA) and/or be a protein in nature (protein, polypeptide).

Whether the expression product of the gene is an RNA or a protein, the method of the invention may comprise or consist of carrying out the steps consisting of:
measuring the quantity of at least one expression product of the VEGFR2 gene in said biological sample from the patient,
comparing the quantity of said at least one expression product determined for said biological sample, or a value derived from this quantity, with a predetermined reference value, and
drawing a conclusion regarding the risk of complications from the result of the comparison.

The various steps of the above method are carried out sequentially.

The measurement of the quantity of at least one expression product of the VEGFR2 gene is carried out as described above. For convenience, the biological sample obtained from a patient for whom the risk of complications is to be assessed will be called the test sample.

In the second step of the method, the quantity of said at least one expression product or a value derived from this quantity will be compared with a predetermined reference value in order to assess the risk of complications.

The determination of a reference value is well known to the person skilled in the art. In particular, it consists of carrying out an assay method which is identical to or at least comparable to that carried out in the method of the invention, in biological samples from two studied populations, and of determining the test value (quantity) which can be used to enable a distinction to be made between these two populations, in the present case between that for which complications will arise and that for which complications will not arise.

A value derived from the quantity may, for example, be the absolute concentration, calculated by means of a calibration curve.

The predetermined reference value used to compare the quantity measured in the context of the invention will be determined from the same expression product or products for the VEGFR2 gene as that or those quantified in the biological sample to be tested.

The samples from which the reference values are determined, also called the “reference samples”, may be of different natures and in particular of a biological nature as mentioned above in respect of the test sample (biological fluids). Advantageously, these reference biological samples are of the same nature as that of the biological sample to be tested, or at least of a compatible nature, in order to constitute a reference for the quantification of the selected expression product or expression products of the VEGFR2 gene. As an example, these will be biological samples corresponding to the same biological fluid as that of the test sample, such as whole blood, serum or plasma samples. The reference sample or samples used are preferably obtained from individuals having the same characteristics or a majority of characteristics in common, in particular of the same sex and/or similar or identical age and/or of the same ethnic origin, as those of the subject or patient suspected of having an infection without sepsis in whom the risk of complications is to be assessed.

In contrast, the reference sample or samples used will be obtained from patients suspected of an infection and having a SOFA score of less than two at the time the biological sample is taken. Their development or not into complications will nevertheless have been documented a posteriori in order to know whether they belong to the population in which complications develop or to that in which complications do not develop.

In order to determine the reference value and in order to quantify said at least one expression product of the VEGFR2 gene in the test sample, samples that are taken at the same time are preferably used, i.e. as soon as the patient is characterized as being suspected of having an infection, having a SOFA score of less than two, or 24 h later at the latest.

It is known that in general, the results of analyte measurement tests depend to a large extent on the characteristics of the binding partner or partners used. Thus, in the case of the quantification of proteins or polypeptides with the aid of a binding partner, such as antibodies, the results in particular depend on the characteristics of the partners used such as the nature, the degree of affinity with the analyte or the size, the characteristics of the compositions, etc., and whether these characteristics have an influence on the measured values. Thus, it will be realized that it is not possible to provide precise reference values and that the reference value or values adapted to the test employed may be determined in each case by simple routine experiments. This is also the case with molecular detection.

The determination of the reference value in accordance with the invention is carried out on a significant sample of patients, i.e. on a minimum number of samples to obtain statistically pertinent results and therefore results that represent the population being studied.

The person skilled in the art will know how to determine the reference value with which the quantity of said at least one expression product determined for said biological sample or the value derived from this quantity used for the comparison may be compared, as a function of the comparison to be carried out. It should be understood that the term “reference value” is used either for a discrete value or for a range of values corresponding to a zone of uncertainty. Clearly, when the measured value is included in the range of uncertainty, or is very close to the reference value in the case of a discrete value, a definitive conclusion cannot be drawn and supplemental investigations should be carried out.

The determination of the reference value or test value (quantity) meaning that a distinction can be made between these two populations may be calculated using the ROC curve (Receiver Operating Characteristic Curve). This curve is a graph obtained by plotting the fraction of false positives, i.e. the Specificity as defined below, along the abscissa and by plotting the fraction of false positives, i.e. the Sensitivity as will be defined below, up the ordinate for different fixed threshold values. It represents all of the Sensitivity/Specificity pairs when the decision threshold varies over the range of values observed in the test. An overall way of quantifying the diagnostic effect of a test is to express its performance as the area under the ROC curve. By convention, this area is always ≥0.5. The values for the area under the ROC curve vary between 0.5 (no difference in the distribution of values for assay between the two sub-groups: the ROC curve corresponds to the bisector) and 1 (complete separation of the assay values for the two sub-groups; the ROC curve passes through the point (0, 1). The area under the ROC curve is a quantitative expression of the position of the ROC curve with respect to the point (0, 1) (Hanley, J. A. and McNeil, B. J, 1982; Zweig, M. H. and Campbell G., 1993).

The sensitivity represents the percentage of True Positives in the totality of Positives recognized as such. It expresses the ability of the test to detect genuinely positive biological samples which correspond to the pathology. In “probabilistic” terminology, it corresponds to the probability of observing a Positive result knowing the Positive sample.

The specificity represents the percentage of False Negatives in the totality of Negatives, recognized as such. It expresses the ability of the test not to diagnose genuinely negative samples, which corresponds to a health individual. In “probabilistic” terminology, it corresponds to the probability of observing a Negative result knowing the Negative sample.

In accordance with the method of the invention, when the value for the quantity of said at least one expression product of the VEGFR2 gene is less than the reference value, then the patient suspected of having an infection who is being tested is a patient at increased risk of complications. In contrast, when the value for the quantity of said at least one expression product of the VEGFR2 gene is higher than said reference value, this signifies that the patient suspected of having an infection who is being tested is not a patient at increased risk of complications. A “patient with an increased risk” has been defined above.

The determination of the risk of complications may also be carried out by measuring the quantity of at least one expression product of the VEGFR2 gene in a biological sample to be tested at two different times.

Thus, in a particular embodiment of the invention, the method may comprise or consist of carrying out the following steps consisting of:
measuring a first quantity of at least one expression product of the VEGFR2 gene in said patient's biological sample obtained by taking a first sample at time T1,
measuring a second quantity of said at least one expression product of the VEGFR2 gene in said patient's biological sample obtained by taking a second sample at time T2,
calculating the variation between the quantity of said at least one expression product of the VEGFR2 gene at T2 and the quantity of said at least one expression product of the VEGFR2 gene at T1, giving a value Δ,
comparing the value 4 obtained in the preceding step with a reference value determined from two populations of patients suspected of having an infection, having a SOFA score of less than two, one developing complications and the other not,
drawing a conclusion regarding the risk of complications from the result of the comparison.

Preferably, the first sample at time T1 will be taken in the 12 hours which follow the identification of the patient as being suspected of having an infection without sepsis and the second sample at time T2 will be taken in the 24 hours (T24) which follow the first sample at time T1.

After determination of the second quantity, this particular embodiment of the invention comprises a step for calculating the variation between the quantity of expression product of the VEGFR2 gene at T2 and that at T1, giving a value Δ.

The calculation of the value Δ may be carried out using any calculation that is known to the person skilled in the art for demonstrating a difference between a quantity at T1 and a quantity at T2.

By way of examples, the following three ways of calculating the value Δ may be cited:
in accordance with the following formula (I):

(VEGFR2 at T1)−(VEGFR2 at T2) (I).

In this case, the value Δ has the same units as the determined quantity (VEGFR2 at T1) or (VEGFR2 at T2).
the value Δ may correspond to the relative variation and is calculated in accordance with one of the following formulas (II) or (II)′:

$$\begin{matrix}\frac{VEGFR 2 at T 1 - VEGFR 2 at T 2}{VEGFR 2 at T 1} or & ( II ) \\ \frac{VEGFR 2 at T 1 - VEGFR 2 at T 2}{VEGFR 2 at T 1} \times 100 & {( II )}^{′}\end{matrix}$$

In this case, the value Δ is either a ratio (II) or a percentage (II)'.
the value Δ may correspond to the difference in quantity per unit time and is calculated in accordance with the following formula (III):

$$\frac{VEGFR 2 at T 2 - VEGFR 2 at T 1}{T 2 - T 1} \qquad ( III )$$

In this case, the value Δ is in the units of quantity per unit time.

The method comprises a step for comparison of the value Δ, obtained in the preceding step, with a reference value determined from two populations of patients suspected of having an infection, having a SOFA score of less than two, one developing complications and the other not.

The reference value is determined as indicated above. In this context, it also requires two times for taking reference samples.

The method in accordance with this embodiment means that a conclusion can be drawn as to the level of the risk of complications in the patient from whom the biological sample was taken, a value Δ lower than said reference value signifying that the patient being tested is a patient at increased risk of complications, and a value Δ higher than said reference value signifying that the patient being tested is not a patient at increased risk of complications. A “patient at increased risk” is as defined above.

The method of the invention may be improved by also measuring the level of expression of at least one expression product of at least one other gene, in addition to measuring the level of expression of at least one expression product of the VEGFR2 gene. Thus, the combination of at least two markers means that the specificity and sensitivity of the method for assessing the risk of complications can be improved.

Thus, one embodiment of the invention also comprises or consists of measuring the level of expression of at least one expression product of the uPAR gene.

All of the indications and preferences mentioned above concerning the quantification of the selected expression product or products of the VEGFR2 gene apply equally to the uPAR gene, irrespective of whether the quantification is carried out in a test sample or in a reference sample.

As was the case with VEGFR2, the expression product or products of uPAR (SEQ ID No 6) in the context of the invention, may be any biological molecule obtained by expression of one of these genes. The following may be cited by way of example: an RNA transcript, a protein or a polypeptide.

The uPAR gene (urokinase-type plasminogen activator), also termed PLAUR (it shall solely be termed uPAR below), codes for the receptor of the activator of plasminogen urokinase and, taking into account its role in the localization and promotion of the formation of plasmin, probably influences many processes in the healthy patient and sick patient linked to the activation of cell surface plasminogen and to the localized degradation of the extracellular matrix. It is linked to both the pro-protein and to mature forms of the activator of plasminogen urokinase and enables activation of the pro-enzyme bound to the receptor via the plasmin. Several transcription variants result from alternative splicing of this gene (NCBI Reference Sequence Database, July 2008).

The Ensembl (GRCh38.p10) database identifies 16 transcripts obtained from the transcription of the uPAR gene. These transcripts are identified in Table 2 below.

TABLE 4

| Name of | Identification | Theoretical | |
|---|---|---|---|
| transcript | number | protein size | Sequences |
| PLAUR-201 | ENST00000221264.8 | 290 aa | SEQ ID No7 |
| PLAUR-203 | ENST00000340093.7 | 335 aa | SEQ ID No8 |
| PLAUR-202 | ENST00000339082.7 | 281 aa | SEQ ID No9 |
| PLAUR-214 | ENST00000601723.5 | 286 aa | SEQ ID No10 |
| PLAUR-207 | ENST00000593939.5 | 152 aa | — |
| PLAUR-213 | ENST00000599892.5 | 226 aa | — |
| PLAUR-212 | ENST00000599546.1 | 78 aa | — |
| PLAUR-205 | ENST00000593447.5 | 133 aa | — |
| PLAUR-216 | ENST00000602141.5 | 152 aa | — |
| PLAUR-209 | ENST00000595038.5 | 154 aa | — |
| PLAUR-206 | ENST00000593714.5 | 185 aa | — |
| PLAUR-210 | ENST00000597107.1 | 46 aa | — |
| PLAUR-208 | ENST00000594364.1 | No protein | — |
| PLAUR-204 | ENST00000593396.1 | No protein | — |
| PLAUR-215 | ENST00000601876.1 | No protein | — |
| PLAUR-211 | ENST00000598875.1 | No protein | — |

Three isoforms of this receptor have been identified, isoform 1 (SEQ ID No 11), No UniProt Q03405-1, also termed uPAR1 or in fact GPI-anchored, which is the membrane form, and isoforms 2 (SEQ ID No 12), No UniProt Q03405-2, and 3 (SEQ ID No 13), No UniProt Q03405-3, which are the secreted plasma forms, or soluble forms, of the receptor. The soluble forms of uPAR are known as suPAR.

As was the case for the VEGFR2 gene, in the context of the present invention, said at least one transcript of the uPAR gene which is measured is selected from the transcripts mentioned in Table 2 and their variants, the sequence of a variant having at least 99% identity with one of the sequences of said transcripts. The percentage identity is determined by means of sequence alignment software such as CLUSTALW. In particular, a variant will correspond to a polymorphism in the sequence of the selected gene.

In identical manner, in the context of the present invention, all of the isoforms obtained from the uPAR gene, referenced above or not yet identified, may be used as a marker to assess the risk of complications in a patient suspected of having an infection, having a SOFA score of less than two.

The binding partners for the isoforms of this marker are of the same nature as those described for VEGFR2.

Antibodies of this type for the isoforms of uPAR are commercially available such as, for example, the following polyclonal antibody: Human uPAR Biotinylated Antibody ref: BAF807 Bio-Techne®, and the following monoclonal antibodies: Human uPAR Antibody ref: MAB807 Bio-Techne®.

In identical manner to the marker or markers cited in combination with VEGFR2, when there are a plurality of measured expression products, they may be of the same nature but may also be of different natures. In other words, they may be molecular in nature (RNA transcript, mRNA) and/or may be a protein in nature (protein, polypeptide).

In accordance with one embodiment, when the uPAR marker is used, the method of the present invention comprises or consists of carrying out the steps consisting of:
measuring the quantity of at least one expression product of VEGFR2 in said biological sample from the patient,
measuring the quantity of at least one expression product of uPAR in said biological sample from the patient,
comparing the quantity of said at least one expression product of the VEGFR2 gene determined for said biological sample or a value derived from this quantity, with a predetermined reference value S_{VEGFR2}; and
comparing the quantity of said at least one expression product of the uPAR gene determined for said biological sample or a value derived from this quantity, with a predetermined reference value S_{uPAR},
drawing a conclusion regarding the risk of complications from the result of the comparisons.

The various steps for measuring the quantity may be carried out sequentially or simultaneously. Similarly, the various steps for comparisons with the reference values may be carried out sequentially or simultaneously.

The steps for determining the quantities of expression products of these markers and determining the reference value for each marker are carried out as described above.

Similarly, the steps for comparison with the reference values are carried out as described above.

The method in the context of this embodiment can be used to draw a conclusion as to an increased risk of complications in a patient suspected of having an infection, having a SOFA score of less than two, when the value for the quantity of said at least one expression product of the VEGFR2 gene is less than the reference value S_{VEGFR2} and the value for the quantity of said at least one expression product of the uPAR gene is higher than the reference value S_{uPAR}. This same method can be used to conclude that the patient tested, suspected of having an infection and having a SOFA score of less than two, is not a patient at increased risk of complications when the value for the quantity of said at least one expression product of the VEGFR2 gene is higher than the reference value S_{VEGFR2} and the value for the quantity of said at least one expression product of the uPAR gene is lower than the reference value S_{uPAR}.

As indicated above, the determination of the risk of complications may also be carried out by measuring the quantity of the expression products of different markers in a biological sample to be tested at different times. In addition, another embodiment is a method which comprises or consists of carrying out the steps consisting of:
measuring a first quantity of at least one expression product of the VEGFR2 gene in said patient's biological sample obtained by taking a first sample at time T1,
measuring a second quantity of said at least one expression product of the VEGFR2 gene in said patient's biological sample obtained by taking a second sample at time T2,
measuring a first quantity of at least one expression product of the uPAR gene in said patient's biological sample obtained by taking a first sample at time T1,
measuring a second quantity of said at least one expression product of the uPAR gene in said patient's biological sample obtained by taking a second sample at time T2,
calculating the variation between the quantity of said at least one expression product of the VEGFR2 gene at T2 and the quantity of said at least one expression product of the VEGFR2 gene at T1, giving a value Δ_{VEGFR2},
calculating the variation between the quantity of said at least one expression product of the uPAR gene at T2 and the quantity of said at least one expression product of the uPAR gene at T1, giving a value Δ_{uPAR},
comparing the value Δ_{VEGFR2} with a reference value ΔS_{VEGFR2} determined from two populations of patients suspected of having an infection, having a SOFA score of less than two, one developing complications and the other not,
comparing the value Δ_{uPAR} with a reference value ΔS_{uPAR} determined from two populations of patients suspected of having an infection, having a SOFA score of less than two, one developing complications and the other not,
drawing a conclusion regarding the risk of complications from the result of the comparisons.

All of the steps for measuring quantity, calculations of variations and comparisons with reference values are carried out as described above.

The various steps for measuring quantity, the various steps for calculating the variations and the various steps for comparisons with the reference values with respect to the various markers may be carried out sequentially or simultaneously provided that, of course, the chronology of: a measurement at T1, measurement at T2, comparison with a reference value and drawing a conclusion, is adhered to for each marker. As an example, the measurement of the quantity uPAR may be carried out from a first sample taken at the time the patient is identified as being suspected of having an infection and the measurement of the quantities of VEGFR2 from a first sample taken 6 hours after the patient has been identified as being suspected of having an infection.

As before, the first samplings at time T1 may have been taken in the 12 hours following identification of a patient as being suspected of having an infection, and the second samplings at time T2 may have been taken in the 24 hours (T24) following the first samplings at time T1.

The sample times T1 and T2 for the marker uPAR may be identical to those for VEGFR2, but they may also be different.

The method in the context of this embodiment allows a conclusion to be drawn as to an increased risk of complications in a patient suspected of having an infection, having a SOFA score of less than two, when the value Δ_{VEGFR2} is lower than said reference value ΔS_{VEGFR2} and when the value Δ_{uPAR} is higher than the reference value ΔS_{uPAR}. This same method can be used to conclude that the patient suspected of having an infection, having a SOFA score of less than two is not a patient at increased risk of complications when the value Δ_{VEGFR2} is higher than said reference value ΔS_{VEGFR2} and when the reference value ΔS_{uPAR} is lower than the reference value ΔS_{uPAR}.

The determination of the risk of complications may also be carried out by using the calculation of a score linked to the various markers used. In addition, the method in accordance with this particular embodiment comprises or consists of carrying out the steps consisting of:
measuring the quantity of at least one expression product VEGFR2 in said biological sample from the patient,
measuring the quantity of at least one expression product uPAR in said biological sample from the patient,
calculating a combined score from the quantities determined in the preceding steps,
comparing the combined score with a predetermined reference score, and
drawing a conclusion regarding the risk of complications from the result of the comparison.

The steps for measuring the quantities of expression product for these markers are carried out as described above and may be carried out sequentially or simultaneously.

The score may be a multiplication type combination, ratio or threshold with different weighting for the at least two markers.

The various quantified markers may also be combined by means of various mathematical algorithms which are well known to the person skilled in the art.

The reference score, or threshold value, may be a multiplication of the reference values determined for each of the marker markers, a division of these reference values when the combination is a ratio, or in fact an equation of the type y=ax+by incorporating different weightings for each marker.

The method in accordance with this particular embodiment allows a conclusion to be drawn of an increased risk of complications in a patient suspected of having an infection, having a SOFA score of less than two when the combined score is higher than a reference score and allows a conclusion to be drawn that the patient suspected of having an infection, having a SOFA score of less than two is not a patient at increased risk of complications when the combined score is lower than the reference score.

In accordance with another embodiment of the above method, the steps for calculation and comparison of the score may be replaced by drawing up a decision tree.

The decision tree in the method of the invention is a tool to aid in making a decision, representing a set of choices in the graphical form of a tree. The various possible decisions are located at the ends of branches (the “leaves” of the tree) and are reached as a function of decisions taken in each step.

The person skilled in the art will readily be able to construct this type of decision tree. Here is an exemplary construction of a decision tree:

If:
concentration of transcript KDR-201 (VEGFR2)<X picoM, and
concentration of transcript PLAUR-203 (uPAR)>Y picoM.
   then the patient is considered to be at risk of complications.

The invention also concerns a method for the treatment of patients based on the risk assessment provided by the methods described above.

Thus, the invention also concerns a method for the treatment of a patient suspected of having an infection, having a SOFA score of less than two, characterized in that it additionally comprises or consists of the steps consisting of:
identifying the patients presenting a risk of complications by carrying out a method for the in vitro or ex vivo assessment of the risk of complications in a patient suspected of having an infection, having a SOFA score of less than two in accordance with the invention and,
adapting the health care management of said patient identified in the preceding step in order to reduce the risk of complications.

A patient identified as being at increased risk of complications may receive health care management that is adapted with the aim of reducing the risk of complications and, for example, of reducing the risk of developing sepsis, septic shock or in fact the risk of death.

Examples of care management that may be cited are an immunostimulant treatment, or indeed a prophylactic antibiotic treatment, the two treatments possibly being associated and/or oriented towards a continuous care unit or intensive care unit in order to reduce the risk of complications, for example to reduce the risk of developing sepsis, septic shock or even the risk of death in the days following the measurement of the level of expression of the marker or markers.

Non-limiting examples of appropriate immunostimulant treatments for preventing the risk of complications are treatment with GM-C SF, IL7, IFNy or in fact anti-PD1.

Appropriate examples of prophylactic antibiotic treatments for preventing pneumonia are described in particular in Annales Françaises d'Anesthesie et de Reanimation (30; 2011; 168-190).

Conversely, a patient not presenting a risk of complications could rapidly be directed towards an outpatient hospital unit, for example an infectiology unit, rather than remaining in a unit with close monitoring that will not be required.

In order to carry out the methods of the invention, when at least one expression product of the VEGFR2 gene and at least one expression product of the uPAR gene are used as markers, the invention also pertains to a kit for predicting the risk of complications in a patient suspected of having an infection, having a SOFA score of less than two, comprising at least one specific binding partner for at least one expression product of the VEGFR2 gene and at least one specific binding partner for at least one expression product of the uPAR gene.

In particular, the invention concerns kits for measuring the level of expression, in vitro or ex vivo, of at least one expression product of the VEGFR2 gene and at least one expression product of the uPAR gene, in a biological sample, comprising:
specific tools or reagents enabling the quantities of said at least one expression product of the VEGFR2 gene and of said at least one expression product of the uPAR gene in said biological sample to be measured, and
a control calibrated to contain the quantities of said at least one expression product of the VEGFR2 gene which correspond to known quantities of said at least one expression product of the VEGFR2 gene, and
a control calibrated to contain the quantities of said at least one expression product of the uPAR gene which correspond to known quantities of said at least one expression product of the uPAR gene.

A control contains a known quantity of one or more expression products of the marker or markers cited in the present application. Preferably, the control contains a known quantity of one or more expression products of a single one of the markers cited in the present application.

This control may be either a synthetic sample containing a calibrated quantity of expression product(s) of the gene or genes of interest, or a biological sample for which the quantity or quantities of expression product(s) of the gene or genes of interest are known.

In accordance with a particular embodiment, said specific binding partner for an expression product of the kit in accordance with the invention is at least one hybridization probe and/or at least one amplification primer, or at least one antibody, or at least one antibody fragment, or at least one affinity protein, or at least one aptamer.

The invention also encompasses the use of a kit in accordance with the invention for carrying out the method of the invention, and in particular to predict the risk of complications in a patient suspected of having an infection, having a SOFA score of less than two.

All of the embodiments which are mentioned above concerning the methods, the objectives of the present invention, all of the characteristics and their combinations apply to the kit in accordance with the invention and to its use.

In accordance with a particular embodiment, said specific binding partner for an expression product of the kit in accordance with the invention is at least one hybridization probe and/or at least one amplification primer or at least one antibody or at least one antibody fragment, or at least one affinity protein, or at least one aptamer.

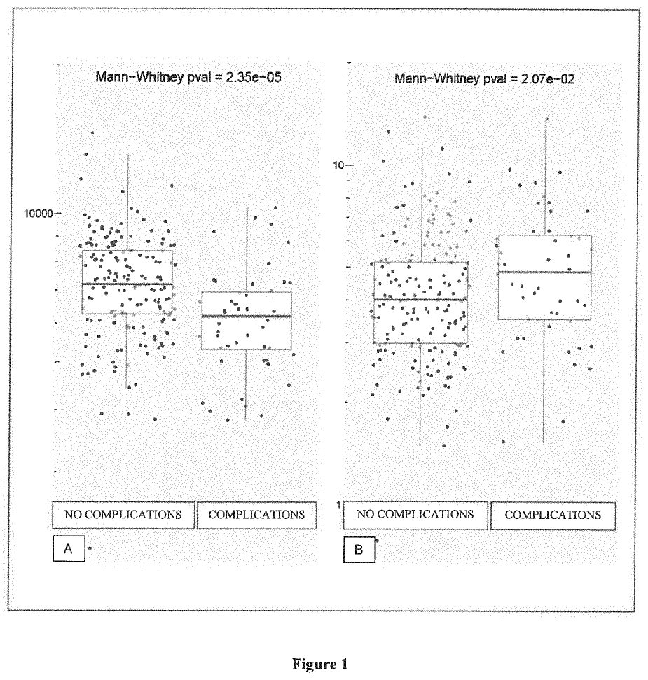

The invention will be better understood with the aid of the following examples which are given by way of non-limiting illustration, as well as with the aid of FIGS. 1 to 4, in which:

FIG. 1 represents box and whisker plots (or box diagrams) which correspond to graphical representations of the levels of expression of sVEGFR2 and suPAR proteins in a sample taken upon admission to the emergency center (T0) as a function of the development or not of complications in the patients: A. Levels of expression for sVEGFR2 at T0; B. Levels of expression for suPAR at T0;

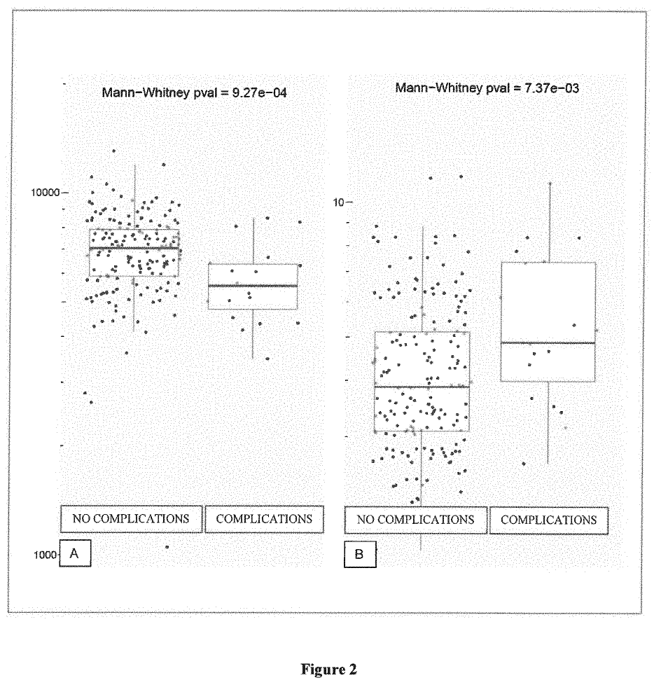

FIG. 2 represents box and whisker plots (or box diagrams) which correspond to graphical representations of the levels of expression of the proteins sVEGFR2 and suPAR in a sample taken six hours after the first sample (T6) as a function of the development or not of complications in patients: A. Levels of expression for sVEGFR2 at T6; B. Levels of expression for suPAR at T6;

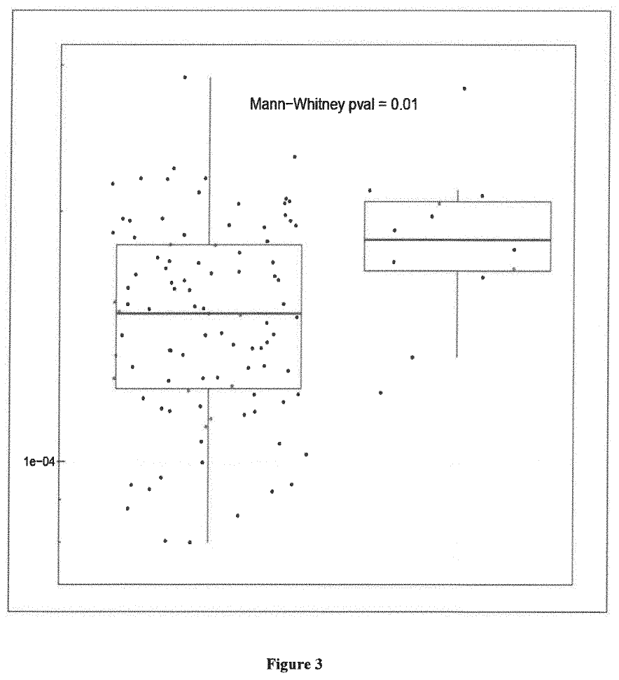

FIG. 3 represents box and whisker plots (or box diagrams) which correspond to graphical representations of the variation in the level of expression of the marker sVEGFR2 between the first blood sample upon admission to the emergency center (T0) and the second blood sample six hours after the first sample (T6) as a function of the development or not of complications in patients.

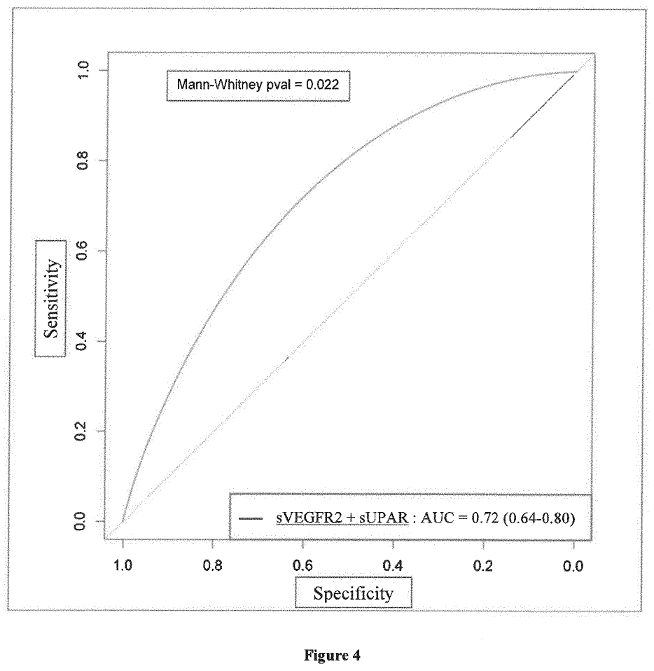

FIG. 4 represents the apparent ROC curve for the association between the combination of sVEGFR2 and suPAR at T0 (first blood sample upon admission to the emergency center) and the probability of complications for patients during the 72 h following the first sample at T0.

### EXAMPLES

### Example 1

### Obtaining and Preparing Blood Samples

This retrospective observational study was carried out on patients from 19 to 101 years of age (111 men, 122 women, median age: 53 years) newly admitted to the emergency center of 14 French and Belgian hospital centers between 2015 and 2017 for care in respect of a suspected infection. These patients were all hospitalized upon suspicion of an infection. The inclusion criteria were as follows:
patients aged 18 years or over;
patients having a single site of acute infection suspected or confirmed by the clinician on clinical or paraclinical signs;
patients admitted to the emergency center presenting at least two criteria from the following:
   temperature higher than 38° C. or lower than 36° C.;
   heart rate more than 90 beats per minute;
   respiratory rate more than 20 breaths per minute or PaCO2<32 mmHg;
   number of leukocytes more than 12000/mm3 or less than 4000/mm3.
patients having a SOFA score of less than 2;
patients having symptoms for less than 72 h from their arrival in the emergency center;
persons affiliated to a social security system or a beneficiary of a system of this type; and
patients consenting to participation in the study.

The clinical criteria for exclusion were as follows:
patients arrived at the emergency center more than 12 hours ago;
patients presenting septic shock upon arrival in the emergency center (organ failure and persistent hypotension despite adequate vascular fluid replacement (up to 20 ml/kg over 1 h) and/or the need to use catecholamines);
patients having acute organ failure upon arrival in the emergency center of an origin other than septic;
patients hospitalized in the week preceding inclusion;
immunosuppressed patients (HIV, transplants, patients undergoing chemotherapy, patients receiving a treatment of >20 mg/jour of prednisolone or equivalent);
patients with a known pathology from among non-infectious pathologies potentially associated with SIRS;
patients with sepsis diagnosed in the 30 days before the date of ‘inclusion;
patients who are already included in the study;
persons refusing to sign the written consent form
adult persons under legal protection;
women who are pregnant, in labor or lactating;
persons with a legal guardian;
person deprived of liberty by a judicial or administrative decision and person hospitalized without consent under articles L.3212-1 and 3213-1 who do not fall within article L.1122-8 of the Public Health Code.

233 patients were included in the study. All of these patients satisfied the following criteria:
the first blood sample was taken at the latest in the first 12 hours following the patient's arrival in the emergency center (T0);
the second blood sample was taken between 4 and 8 hours (T6±2 h) following taking the first blood sample (T0);
the assessment of complications was observed by an adjudication committee in the 72 hours (T72) following taking the first blood sample (T0); and
mortality was assessed 28 days following the patient's arrival in the emergency center.

Complications were determined by an adjudication committee composed of 3 physicians who were independent of the study. This committee determined the complications as a function of several criteria; in particular, the appearance of new organ failures (increase in SOFA score), death or the need to go into intensive care.

Of the 233 patients in this cohort, 36 patients (21%) developed complications in the 72 h following their admission (“COMPLICATIONS”) and 185 patients (79%) did not develop complications (“NO COMPLICATIONS»).

### Example 2

### Assay of the Soluble Form of the VEGF Receptor (sVEGFR2)

Human plasma was collected from patients described above in Example 1 at T0 and T6.

The protein sVEGFR2 was assayed with the aid of antibodies marketed by Bio-Techne® (Ac monoclonal anti Human VEGFR2 (KDR) ref: MAB3573, and Human VEGF R2/KDR/Flk-1 Antibody Antigen Affinity-purified Polyclonal Goat IgG ref: AF357) and an ELISA test using the automated instrument Vidas® (bioMérieux). To this end, the ELISA test was constructed using reagents from the cartridge of the Vidas® B.R.A.H.M.S. PCT™ kit (bioMérieux, Cat. No.30450) without using the antibodies and the control calibrators.

VIDAS® is an automated multi-parametric immunoanalyzer. It is a closed system for unitary tests offering great flexibility. This automated instrument is characterized by its robustness, its flexibility, its ease of use and is intended for small to medium sized laboratories. It can be used to carry out routine tests, confirmations and high value medical tests.

Detection was carried out using the ELFA (Enzyme Linked Fluorescent Assay) technique in serum or plasma. The principle of ELFA assay corresponds to a combination of immunoenzymatic reactions with detection of the end point using fluorescence. The enzyme used is the alkaline phosphatase that catalyzes the hydrolysis reaction of the substrate, 4-méthyl-ombelliferyl phosphate, to a product: 4-méthyl-ombellierone. The product emits at a wavelength of 450 nm after excitation at 370 nm. The results were automatically analyzed by VIDAS® and expressed as the relative fluorescence intensity or RFV (for “Relative Fluorescent Value”). This value for RFV was determined by subtracting the value for the background noise (BKG) from the gross value obtained.

The reagents were used as described in the notes, with the following modifications:
1. The cones were sensitized with MAB3573 monoclonal antibodies in a concentration of 2.5 μg/ml (indirect coating with a first anti-mouse Ac at 10 μg/ml then anti-sVEGFR2 Ac at 2.5 μg/ml);
2. The contents of the fourth well of the cartridge of the Vidas® B.R.A.H.M.S. PCT™ kit was replaced by 400 μl of revealing antibody (ref.: AF357), coupled to biotin, diluted to 1 μg/ml;
3. The plasma samples (200 μl) were used directly, pure;
4. The ELISA reaction was carried out with the aid of the automated Vidas® instrument and application of the Vidas® B.R.A.H.M.S. PCT™ kit protocol;
5. The results were obtained in the form of gross values after subtracting the background noise (reading for substrate before reaction). A calibration curve was produced by assaying a range of concentration for the marker in the form of recombinant protein (Recombinant Human VEGF R2/KDR/Flk-1 Fc Chimera. Bio-Techne® ref: 357-KD-050). The calibration curve was plotted by recording the concentration of marker along the abscissa and the signal read by Vidas® (RFV or Relative Fluorescence Value) up the ordinate. The concentration of marker present in the serum was calculated by recording the concentration corresponding to the RFV signal read by Vidas®.

### Example 3

### Assay of the Soluble Form of the Receptor for uPA (suPAR) by ELISA

Human serum was collected at from patients described in Example 1 T0 and T6.

The suPAR blood counts were measured with the aid of frozen serum (samples stored at −80° C.). The samples were analyzed using the commercial CE/IVD marked ELISA suPARnostic® AUTO Flex kit, in accordance with the instructions of the manufacturer (Virogates, Birkeroed, Denmark). The suPARnostic® ELISA test is based on a simplified double monoclonal antibody sandwich ELISA assay in which serum samples and peroxidase-conjugated anti-suPAR are initially mixed then incubated in anti-suPAR pre-coated micro-wells. The recombinant suPAR standards of the kit were calibrated and enabled a calibration curve to be calculated. The concentrations of suPAR were determined in ng/ml of plasma. The test was validated for measuring suPAR levels between 0.6 and 22 ng/ml.

### Example 5

### Statistical Analyses

The statistical analyses were carried out using R software, version 3.4.0. The differences observed were considered to be significant for values of p, or p-values, of less than 0.05.

Association Between the Level of Expression of Markers sVEGFR2 and suPAR and the Appearance or Otherwise of Complications

The predictive capacity of measuring the level of expression of the markers was studied having regard to the appearance or not or complications in the patients in the 72 hours which followed taking the first blood sample at T0. The Wilcoxon-Mann-Whitney test was used to characterize this association.

The levels of expression of VEGFR2 and suPAR at T0 and T6 were measured as described above in blood samples from 233 patients suspected of having an infection, having a SOFA score of less than two. The results are presented in Table 5 and in FIGS. 1 and 2.

TABLE 5

Mann-Whitney (p-value)

| | sVEGFR2 | suPAR |
|---|---|---|
| T0 | <0.0001 | 0.02 |
| T6 | <0.001 | 0.00737 |

The results shown in FIGS. 1 and 2 provide, up the ordinate, the level of expression of sVEGFR2 (in pg/mL) and suPAR (in ng/mL) at T0 and T6, as a function of the development or not of complications in patients.

These results showed a significant association (p<0.05) between the level of expression of the markers sVEGFR2 and suPAR at T0 and at T6 and complications in the 72 hours following taking the first blood sample at T0. The sVEGFR2 protein exhibited better performances at T0 and T6 than suPAR.

The level of expression of the studied markers can thus be used to distinguish patients who will develop complications in the 72 hours which follow taking the first blood sample at T0 from those who will not develop complications.

More precisely, the patients in whom complications will appear had lower levels of expression of sVEGFR2 than patients who did not suffer from any complications. Concerning suPAR, the patients in whom complications will develop had higher levels of expression than patients who did not suffer from any complications.

Association Between the Variation in the Level of Expression of sVEGFR2 and the Development or Not of Complications

Apart from the association between the level of expression of sVEGFR2 and the development of complications, the association between the difference in the level of expression of sVEGFR2 measured in two successive samples taken between 4 and 8 hours and the appearance of complications in the 72 hours which followed taking the first sample was observed.

The level of expression of sVEGFR2 at T0 and T6 was measured as described above in the plasma samples from 233 patients suspected of having an infection, having a SOFA score of less than two. For each patient, the variation was calculated in accordance with the following formula:

$$Δ = \frac{sVEGFR 2 at T 0 - sVEGFR 2 at T 6}{sVEGFR 2 at T 0}$$

The results are presented in FIG. 3 giving, up the ordinate, the result of a variation between T0 and T6 (Δ in accordance with the above formula), as a function of the development or not of complications in patients, patients for whom clinical monitoring had shown that their condition then deteriorated (“COMPLICATIONS”), and patients for whom clinical monitoring had shown that their condition was not going to deteriorate (“NO COMPLICATIONS”).

These results show that the variation between T0 and T6 of the level of expression of sVEGFR2 is differentially associated (p-value=0.01) with patient complications in the 72 hours which follow the first sample.

More precisely, the patients who saw their condition deteriorate presented a variation in the level of expression of sVEGFR2 between T0 and T6 which was higher than for patients who will not suffer any complications.

Association Between the Level of Expression of sVEGFR2 and suPAR and the Probability of Complications

The association of the variables sVEGFR2 and suPAR with the status of the patient (in the present case «COMPLICATIONS″) was tested by means of a logistic regression. The strength of the association was estimated by calculating Odd Ratios (ORs), which is the ratio of the probability that the patient will develop at least one complication to the probability that the patient will not develop any complications.

For each quantitative variable: level of expression of sVEGFR2 and suPAR, the Odd Ratio was interpreted as follows:
OR=1: no association
OR<1: an increase from the first to the third quartile is associated with a decrease in the risk of complications
OR>1: an increase from the first to the third quartile is associated with an increase in the risk of complications

The IQR is the interquartile range. The IQR is a measure of dispersion that is obtained by taking the difference between the third and the first quartile.

IQR.OR was measured for the blood samples from 233 patients suspected of having an infection, having a SOFA score of less than two.

The logistic regression model was also produced in order to analyze the performances of the ratio of the levels of expression of sVEGFR2 and suPAR. The objective was to demonstrate that the ratio of the markers is significantly associated with the risk of complications in the 72 hours which follow taking the first sample.

The results are given in Table 6 below.

TABLE 6

| Markers | IQR.OR | p-value |
|---|---|---|
| sVEGFR2 | 0.36 (1.7-4.8) | 1.19 · 10⁻⁴ |
| suPAR | 1.49 (1.1-2.1) | 2.09 · 10⁻² |
| sVEGFR2/suPAR | 1.74 (1.3-2.4) | 5.13 · 10⁻⁴ |

The value of IQR.OR for the marker sVEGFR2 over the studied population was 0.36 with a p-value equal to 0.0001. Thus, patients with a low level of expression of sVEGFR2 (first quartile) had a significantly higher probability of complications (2.76 times) than patients with a high level of expression (third quartile).

The value of IQR.OR for the suPAR marker over the studied population was 1.49 with a p-value equal to 0.02. Thus, patients with a high level of expression of suPAR (third quartile) had a significantly higher probability of complications (1.49 times) than patients with a low level of expression (first quartile).

The value of IQR.OR for the ratio between sVEGFR2 and suPAR was 1.74 with a p-value equal to 0.0005. Thus, patients with an expression ratio between sVEGFR2 and suPAR (third quartile) had a significantly higher probability of complications (1.74 times) than patients with a low expression ratio (first quartile).

Analysis of the Predictive Performance of sVEGFR2 and suPAR Alone and in Combination

In order to analyze the predictive performance (sensibility/specificity) of the sVEGFR2 and suPAR markers alone and in combination at T0, the areas under the curve (AUC: Area Under Curve) were calculated; in addition, the maximum specificity, the maximum negative predictive value (NPV) and the maximum positive predictive value were assessed for a minimum imposed sensitivity of 0.90. The results are shown in Table 5 and in FIG. 4.

TABLE 5

| | | | Min imposed sensitivity = 0.90 | | |
|---|---|---|---|---|---|
| | | | Max | NPV | PPV |
| Marker at T0 | AUC | 95% CI | specificity | Max | max |
| sVEGFR2 | 0.70 | 0.614-0.783 | 0.17 | 0.87 | 0.22 |
| suPAR | 0.61 | 0.516-0.701 | 0.18 | 0.87 | 0.22 |
| sVEGFR2 + | 0.72 | 0.641-0.8 | 0.31 | 0.92 | 0.25 |
| sUPAR | | | | | |

The markers exhibited very good overall performances at T0. However, sVEGFR2 had better overall performances at T0 (AUC=0.70). In addition, the results show that the combination of sVEGFR2 and suPAR markers can be used to increase the overall performances of the prognostic test. (cf. FIG. 4).

### BIBLIOGRAPHY

Annales Françaises d'Anesthesie and de Reanimation, 30; 2011; 168-190
Asehnoune K et al., 2014, Hydrocortisone and fludrocortisone for prevention of hospital-acquired pneumonia in patients with severe traumatic brain injury (Corti-TC): a double-blind, multicenter phase 3, randomized placebo-controlled trial, Lancet Respir Med, 2:706-16
Bio-Rad Laboratories, 2006, Real-Time PCR: Applications Guide
Boom R et al., 1990, Rapid and simple method for purification of nucleic acids, J. Clin. Microbiol. 28, 495-503
Bucki R et al., 2005, Inactivation of Endotoxin by Human Plasma Gelsolin, Biochemistry, 44 (28)
Buh Gasparic M et al., 2010, Comparison of nine different real-time PCR chemistries for qualitative and quantitative applications in GMO detection, Anal. Bioanal. Chem. 396, 2023-2029
Bustin S A, 2002, Quantification of mRNA using real-time reverse transcription PCR (RT-PCR): trends and problems, in Journal of molecular endocrinology, 29: 23-39
Chahin A et al., 2015, The novel immunotherapeutic oligodeoxynucleotide IMT504 protects neutropenic animals from fatal Pseudomonas aeruginosa bacteriemia and sepsis, Antimicrob Agents Chemother, 59(2):1225-9
Chee, M et al., 1996, Accessing genetic information with high-density DNA arrays, Science 274, 610-614
Cheng J et al., 1998, Preparation and hybridization analysis of DNA/RNA from E. coli on microfabricated bioelectronic chips, Nat. Biotechnol. 16, 541-546
Cheng J et al., 1996, Mol. Diagn. J. Devoted Underst. Hum. Dis. Clin. Appl. Mol. Biol. 1, 183-200
Clontech, 2003, BD QZyme Assays for Quantitative PCR
Cloonan N. et al., 2008, Stem cell transcriptome profiling via massive-scale mRNA sequencing, Nat. Methods 5, 613-619
Duck P et al., 1990, Probe amplifier system based on chimeric cycling oligonucleotides, BioTechniques 9, 142-148
Eamon P. Raith, et al., Prognostic Accuracy of the SOFA Score, SIRS Criteria, and qSOFA Score for In-Hospital Mortality Among Adults With Suspected Infection Admitted to the Intensive Care Unit. JAMA. 2017; 317(3):290-300. doi: 10.1001/jama.2016.20328
Emrich S J et al., 2007, Gene discovery and annotation using LCM-454 transcriptome sequencing, Genome Res. 17, 69-73
Essader A S et al., 2005, A comparison of immobilized pH gradient isoelectric focusing and strong-cation-exchange chromatography as a first dimension in shotgun, Proteomics, 5, 24-34
Fagon et al., 1993, Characterization of intensive care unit patients using a model based on the presence or absence of organ dysfunctions and/or infection: the ODIN model, Intensive Care Med; 19:137-44
Fortin T et al., 2009, Clinical Quantitation of Prostate-specific Antigen Biomarker in the Low Nanogram/Milliliter Range by Conventional Bore Liquid Chromatography-Tandem Mass Spectrometry (Multiple Reaction Monitoring) Coupling and Correlation with ELISA Tests. Mol. Cell Proteomics, 8(5): 1006-1015
Ginot F, 1997, Oligonucleotide micro-arrays for identification of unknown mutations: how far from reality?, Hum. Mutat. 10, 1-10
Goblet C et al., 1989, One-step amplification of transcripts in total RNA using the polymerase chain reaction, Nucleic Acids Res. 17, 2144
Giulietti A et al., 2001, An overview of real-time quantitative PCR: applications to quantify cytokine gene expression, Methods, 25: 386-401
Hanley J A and McNeil B J, 1982, The meaning and use of the area under a receiver operating characteristic (ROC) curve, Radiology, 143: 29-36
Hellemans J et al., 2007, qBase relative quantification framework and software for management and automated analysis of real-time quantitative PCR data, Genome Biol. 8, R19
Jensen J U et al., 2011, Procalcitonin-guided interventions against infections to increase early appropriate antibiotics and improve survival in the intensive care unit: a randomized trial, Crit Care Med, 39(9):2048-58
Keller G H and Manak M M, 1993. DNA Probes, p. 137-196. Stockton Press, Stockton Press
Kricka L J, 1999, Nucleic acid detection technologies—labels, strategies, and formats, Clin. Chem. 45,453-458
Koch S and Claesson-Welsh L, 2012, Signal transduction by vascular endothelial growth factor receptors. Biochem J., 437(2): 169-183
Lambert M L S C et al., 2011, Clinical outcomes of health-care-associated infections and antimicrobial resistance in patients admitted to European intensive-care units: a cohort study, Lancet Infect Dis, 11:30-8
Levison P R et al., 1998, Recent developments of magnetic beads for use in nucleic acid purification, J. Chromatogr. A 827, 337-344
Levy M M et al., International Sepsis Definitions Conference, Intensive Care Med. April; 29(4):530-8
Livache T et al., 1994, Preparation of a DNA Matrix via an Electrochemically Directed Copolymerization of Pyrrole and Oligonucleotides Bearing a Pyrrole Group, Nucleic Acids Res. 22,2915-2921
Mhlanga M M and Malmberg L, 2001, Using molecular beacons to detect single-nucleotide polymorphisms with real-time PCR, Methods San Diego Calif 25, 463-471
Michel P E et al., 2003, Protein fractionation in a multicompartment device using Off-Gel, isoelectric focusing, Electrophoresis, 24, 3-11
Millauer B et al., High affinity VEGF binding and developmental expression suggest Flk-1 as a major regulator of vasculogenesis and angiogenesis, Cell. 1993; 72:835-846
Mortazavi, A et al., 2008, Mapping and quantifying mammalian transcriptomes by RNA-Seq, Nat. Methods 5, 621-628
Nazarenko I et al., 2002, Multiplex quantitative PCR using self-quenched primers labeled with a single fluorophore, Nucleic Acids Res. 30, e37
Nazarenko I A et al., A closed tube format for amplification and detection of DNA based on energy transfer, 1997, Nucleic Acids Res. 25, 2516-2521
NCBI Reference Sequence Database, July 2008
Oschsner U A et al., 2014, Systematic selection of modified aptamer pairs for diagnostic sandwich assays, BioTechniques, 56: 125-133
Pease A C et al., 1994, Light-generated oligonucleotide arrays for rapid DNA sequence analysis, Proc. Natl. Acad. Sci. U.S.A. 91, 5022-5026
Puisieux F et al., 1993, Prophylactic antibiotherapy using cefapirin in the surgery of duodenal ulcer: a randomized clinical trial, Ann Fr Anesth Reanim, 12(3):289-92
Ramsay G, 1998, DNA chips: condition-of-the art, Nat. Biotechnol. 16, 40-44
Sambrook J and Russell D W, 2017, Purification of Nucleic Acids by Extraction with Phenol:Chloroform, Cold Spring Harb Protoc, doi:10.1101
Shibuya M, 2011, Vascular Endothelial Growth Factor (VEGF) and Its Receptor (VEGFR) Signaling in Angiogenesis, Genes Cancer, 2(12): 1097-1105
Shibuya M et al., 1990, Nucleotide sequence and expression of a novel human receptor-type tyrosine kinase gene (flt) closely related to the fms family, Oncogene. 1990 April; 5(4):519-24
Sigma, 2008, qPCR Technical Guide
Thompson J D et al., 1994, CLUSTAL W: improving the sensitivity of progressive multiple sequence alignment through sequence weighting, position-specific gap penalties and weight matrix choice. Nucleic Acids Res. 11; 22(22):4673-80
Vincent J L & Coll, The SOFA (Sepsis-related Organ Failure Assessment) score to describe organ dysfunction/failure. Intensive Care Med 1996; 22:707-710
Wada T et al., 2013, The role of angiogenic factors and their soluble receptors in acute lung injury (ALI)/acute respiratory distress syndrome (ARDS) associated with critical illness. Journal of Inflammation, 10:6
Zweig M H and Campbell G, 1993, Receiver-Operating Characteristic (ROC) Plots: a fundamental evaluation tool in clinical medicine, Clin. Chem., 39/4: 561-577

1. A method for the assessment, in vitro or ex vivo, of the risk of complications in a patient suspected of having an infection, having a SOFA score of less than two, comprising measuring the level of expression of at least one expression product of the VEGFR2 gene in a biological sample obtained from the patient.

2. The method as claimed in claim 1, wherein the expression product of the gene is an RNA transcript.

3. The method as claimed in claim 1, wherein the expression product is a protein or a polypeptide.

4. The method as claimed in claim 1, comprising:
measuring the quantity of at least one expression product of the VEGFR2 gene in the biological sample from the patient,
comparing the quantity of the at least one expression product determined for the biological sample, or a value derived from this quantity with a predetermined reference value, and
drawing a conclusion regarding the risk of complications from the result of the comparison.

5. The method as claimed in claim 1, comprising:
measuring a first quantity of at least one expression product of the VEGFR2 gene in the patient's biological sample obtained by taking a first sample at time T1,
measuring a second quantity of the at least one expression product of the VEGFR2 gene in the patient's biological sample obtained by taking a second sample at time T2,
calculating the variation between the quantity of the at least one expression product of the VEGFR2 gene at T2 and the quantity of the at least one expression product of the VEGFR2 gene at T1, giving a value Δ,
comparing the value Δ obtained in the preceding step with a reference value determined from two populations of patients suspected of having an infection, having a SOFA score of less than two, one developing complications and the other not,
drawing a conclusion regarding the risk of complications from the result of the comparison.

6. The method as claimed in claim 1, also comprising measuring the level of expression of at least one expression product of the uPAR gene.

7. The method as claimed in claim 6, comprising:
measuring the quantity of the at least one expression product of the VEGFR2 gene in the biological sample from the patient,
measuring the quantity of the at least one expression product of the uPAR gene in the biological sample from the patient,
comparing the quantity of the at least one expression product of the VEGFR2 gene determined for the biological sample or a value derived from this quantity with a predetermined reference value S_{VEGFR2}; and
comparing the quantity of the at least one expression product of the uPAR gene determined for the biological sample, or a value derived from this quantity with a predetermined reference value S_{uPAR};
drawing a conclusion regarding the risk of complications from the result of the comparisons.

8. The method as claimed in claim 6, comprising:
measuring a first quantity of the at least one expression product of the VEGFR2 gene in the patient's biological sample obtained by taking a first sample at time T1,
measuring a second quantity of the at least one expression product of the VEGFR2 gene in the patient's biological sample obtained by taking a second sample at time T2,
measuring a first quantity of the at least one expression product of the uPAR gene in the patient's biological sample obtained by taking a first sample at time T1,
measuring a second quantity of the at least one expression product of the uPAR gene in the patient's biological sample obtained by taking a second sample at time T2,
calculating the variation between the quantity of the at least one expression product of the VEGFR2 gene at T2 and the quantity of at least one expression product of the VEGFR2 gene at T1, giving a value Δ_{VEGFR2},
calculating the variation between the quantity of the at least one expression product of the uPAR gene at T2 and the quantity of at least one expression product of the uPAR gene at T1, giving a value Δ_{uPAR},
comparing the value Δ_{VEGFR2} with a reference value ΔS_{VEGFR2} determined from two populations of patients suspected of having an infection, having a SOFA score of less than two, one developing complications and the other not,
comparing the value Δ_{uPAR} with a reference value ΔS_{uPAR} determined from two populations of patients suspected of having an infection, having a SOFA score of less than two, one developing complications and the other not,
drawing a conclusion regarding the risk of complications from the result of the comparisons.

9. The method as claimed in claim 6, comprising:
measuring the quantity of the at least one expression product of the VEGFR2 gene in the biological sample from the patient,
measuring the quantity of the at least one expression product of the uPAR gene in the biological sample from the patient,
calculating a combined score from the quantities determined in the preceding steps,
comparing the combined score with a predetermined reference score, and
drawing a conclusion regarding the risk of complications from the result of the comparison.

10. A method for the treatment of a patient suspected of having an infection, having a SOFA score of less than two, comprising:
identifying the patients presenting a risk of complications by carrying out a method as claimed in claim 1, and
adapting the health care management of the patient identified in the preceding step in order to reduce the risk of complications.

11. A kit for measuring, in vitro or ex vivo, the level of expression of at least one expression product of the VEGFR2 gene and of at least one expression product of the uPAR gene in a patient suspected of having an infection, having a SOFA score of less than two, comprising at least one specific binding partner for the at least one expression product of the VEGFR2 gene and at least one specific binding partner for the at least one expression product of the uPAR gene.

12. A kit for measuring, in vitro or ex vivo, the level of expression of at least one expression product of the VEGFR2 gene and of at least one expression product of the uPAR gene in a biological sample, comprising:
specific tools or reagents enabling the quantities of the at least one expression product of the VEGFR2 gene and of the at least one expression product of the uPAR gene to be measured in the biological sample, and
a control sample which is a sample calibrated to contain the quantities of the at least one expression product of the VEGFR2 gene and of the at least one expression product of the uPAR gene which correspond to known quantities of the at least one expression product of the VEGFR2 gene, and of the at least one expression product of the uPAR gene.

13. The kit as claimed in claim 11, in which the specific binding partner for an expression product is (i) at least one hybridization probe and/or at least one amplification primer, or (ii) at least one antibody, or at least one antibody fragment, or at least one affinity protein, or at least one aptamer.


SEQUENCE LISTING (13 sequences)

SEQ ID NO: 1 - DNA, 47115 bp, Homo sapiens
actgagtcccgggaccccgggagagcggtcaatgtgtggtcgctgcgtttcctctgcctgcgccgggcatcacttgcgcgccgcagaaagtccgtctggcagcctggatatcctctcctaccggcacccgcagacgcccctgcagccgcggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtaaggagcccactctggaggaggaaggcagacaggtcgggtgagggcggagaggacctgaaagccagatctaactcggaatcgtagagctggagagttggacaggacttgacattttgcgatctttcatttaccagtggggaaactgaggctcagagactggcccaagattacccagcgagtctgtggtcgcctgtgctctagcccagttccttttctaggactctggtttgcgacagggacctcggctggagcatgtcctgagatgccgacacaccctcaggctcttgggaggctggggtgggaaggcgcctggggttggcaggcaggaggtgcctccgcaggcgagaacaggcggtgaaaagttgtctggctgcgcgcaacatcctagtccgggcccggggaagaaaaccttgccggaatctcaggccgggtctcccggatcggacggtacactcggttctgcctctttgcgggacccggcccgttgttgtcttcatgctcgaacacacttgcacaccactgtgtgaagtggggtctggagcggagagaaactttttttccttccttggtgcaggacgccgctctccttgcagagcgaagaagggggggaatagggacttgtcctgggggctttgacagcttccccaagggtctccaagtaacagccaactgtcctgcgtaaagcattgcacatctttcaaagcgctgtggtccttggtgtaagcgcatagtcagaagttcaagctccgaaaacctttcctgtgggccttggtacctagctttagtgccattccttcctctccctgccgcctaaaatttccgtctccttcaattaggaacacacacgttcttcatgcaatagctgtctgtcttttcttcctcactttcctttctctctcaaccccttagataatatttctttcctgcagccagtttgctgatatccagatttccaccctttgcagggtgagaaaggggaaagggtcagagaaagaaaaaaaaaaagtcgaataattcagggaaaaaaatttcttactccctaagacaagaatcacatgtcttagaagacactcacacccacatacagtaccaggatcatctgtccatggttactgaattttctttataatgacttggttcaacgggtccagtccaccatggacactcatttgtcccagacaagccctctctctccccctttctgggcagagaatgaaggtctggaacatgtggttgctctgtattccacaaagaagtgagttgcttttaagcctggggtgtttcctagcgtagtagtaacggcaggccggtcgccctgaatataatggtgaacttgcccttttggagtgcattacttgcttaattggattgggctgtaattggtgccatcaaattctagagacagaggcactgttgtttttccttcccgtctttgagctggaagggtaacagtgcacaaattaattaatattggttatgggatttgaacatagaagggctttttattgagtagtagcatgtgtacctcttacagttatttctttagaactttctgaagagtccagctcaagcttgccaatgaaaacgaatgacatttaatggagcaaaaacaaaaaacaaaaaactatgttggtctacaaatatgaatttgaagttattgagagccttgttgaatagatttttgttgtaaacgtgtctctagaatagtatggcatagtctcagcttcctatgaatgaaggacataccttttcttttttaaaatatttgttacacaggaaagtgtgtctagaatgtgatctgtggcaataaattatgagagaccttcaagagtttctgattttggtagccgagtgggcacagtttattgagaatcatttttactgccatttgttttctcacaagaatgtgcccaaataatggtttttttctcatttggatggcagtgtgaattgtacatcatgttttcagcatctttctcaacctagtgttccccagtcaagtttgaaatctgtgttatccaaatgaattgttttcattttccttttcttagacaaagtgggactccaggtttcattttgcttttaaacattttggttttttgtttgcctgttttgggggcagttatttctttcatattaaaaagtactgtgcaggctgggtgcagtggctcattcctgtaatcccagcacttagggaagcagaggcaggaggatcgcttgagtccaggagttcaagaagtgcctgggcaacatagcgagaccccattctctatttaaaacataaatgtaacccccgttccacgcacaaagtactgtgcaaattaattaaacatgaccacccagaccagcaactgtccaagagtggcccatagaccatctgtggtaggataatttgaaatgcttgttaaaatgcagatttgtagacccagggatattctgacagagtctaaagtcttaagaacaaaactgttctaaacataagtcagtaccaatgccagttaatttctgagatatattgatataacttagtttccagttttttaaaaaccatattattgacttaaaaaccatgatattgaccagttatgtcagtaacttattttgcacatctgtgtggtgtgtgagaacatgtgcagtcacttattcattttgcctgcatttgttcatattgggatcctcagattcaatgcactggatgtttgcactgggtatttacttatactctctctatttattccgtctcatacttcgtcctatttgttcatactctcttatttgcccagcaaggtcaatgccagtttaggcctagggagtcattttttcttagttgatatgacttagaaagcttgggagcctgcccaacatcaattacttttttaaagctggtattttctaggtcttgatatttattaagaccctagcatagtggacaatttttctttctctcatgctttttcaacacctcatagctcttcacatttagttgacagagaattcagttatcttgctgtagagtgacccatggtgaggaatctatgccatggtacttttctggttcttatcccttataggtaaagacaagtttcttatgtctgaagcttgatgtcaggatgagttcagggctttgatgaataagttcagatctcccaattgtaattcattagcattgcacttaaaaaaatttatatacgtttttaaaaaagggtaatgctaatgaattacaatagagagaaaagtacattagtttgcatgtatgtgtgaaactgggaaaatttttcacgaaaatattcatatactttttaaaaaaagggtaatgctaatgaattacagtagacagaaaagtatattaatttgcacatatgtgtaaaattgggaaaattccacacatacataaaagtatattaatatgcatgtatgtgtggaattggggaatgttttctcttcctcagtttctctcccttgcttttaatgtacagtctttatgagccattatttcagctgtggcagtttggttaccaggggaagcgcactagaaaattgataaaggaaaatgagacaaggtcatagattctctcactcccttcagggtacgtagatgaactatataaaaatccgtctaagtgggattcgttaatcagcaatttagtcaaatgtgtacatcctatgttctataagaaatgtcagtgggtcctttcccaagggagtgagatcatcagatgaaggttcatttggtttcaatgtcccgtatccttttgtaagaccttgaagttggcaatgcaggaaaacaggaactccaccctagctccatgaattgcagaactgttgtgttggtttatgaccatctgcccattcttcctgttatgacacagcttgtgaacttttactgagaatggtgaaaagtaaattcccagttttatacaatgaattgctgaagaggccttttaaagtatagagtatgcattgtttatggaaggtgtttcctattaggtctaactcagtggcaactacattcatttatttaatttgtttctaggtttgcctagtgtttctcttgatctgcccaggctcagcatacaaaaagacatacttacaattaaggctaatacaactcttcaaattacttgcaggtaaggattcattctagatctagatttcttgtgttaagtaactgattgtttattgagtggaaataatttccagtagagcagaattataatagagcttgtagtaattgttcataagtggtgaggtttctaagaactgatgtaataatggaaaatgagaagaattttctctcaaaaattctgtacaattttgctggtgtttttatactattctctgccaacatgcatacacacacacacacacacacgcacacaaatacacacccacacccacattccaataaccagtacagccacctggcgtatagtagacatacgctcaataaatatgaatgaataaatgaagttgagggcatacatttaaggaatagagttgaaaaaatttgggactatatttattatgcttggtatgattcttgaacacttattatccctttccaaaaactttgctttataagaaatttattactataattacttaggcagtaatatttaatagcaatttaatatttagtgggtaatattactgagcgcatgatctacataaataatggacttcgggccctgccttgatattctggaatgcatctttccccacttgctagcaagaagtcatgctattgatttttgataactggagaagtagacttctttgtcaagaagaagaggcctttaaattttgcctttcaacccttaccccaggacgaaagatagaagacccttgggtttaacatagtgatcacacacgaaaggcatggagccttcttaggacctgtgtgtttttggtagagactgtgacaagtggaggtgatgttaccctcctggaagagtgctgggggtccacaaaggaccttgggtaggttattgccattgcttcatacttgttgaatactaagcattaaaccgaatgacatacatctattttagactgcagtataaagaataccctagccccttaccaatacccagcccttgggaaaaaacacagtagcaggtgctgtttctctagctttacttgtttaagacacatttcccattagattttccttttaccgaccctcgataacaaggttatttgaaatccccaaggatcccatgctccctttttaaaactctgcataaacatttcttatgttctgaaaaaaaccatggagtgtgttaaaagtaacttcattgatttagctgcaacttcctggaaattttaagttctttgaatgaagggccaataatgttacattcttcttgatgttgactatcttcttatcttccttggggccttgtagagaaatgctgcagtacaagccatctatgttttaatgcgaggtccttacaaggtcctgagggactcttacttgcacctccttccttcctaacctcacttcttactcccctttgctcactcttacctggctgctctggtttcctggctgttcccttaatactccagatatgcacctgctccagggcctttccatgtgctgtttttgctcctgtaatactgctcttcatgatgttcctatggctagctttatcaagaccacctcctgcaaaattctttactcttttctttgtatcttctatatttttctccatagtactaaacactatcttttatacaataaactttccttactttttaattgcctgttttctccagttagactgaggttccataaaggcattgatttttgtctgatttgttcactgctctttctctagtccttaacaagtttggcacatagtagatgcttaatagatatttgttgaaagaaagaatgcattaattaatggaaaactcaggaatctttataagtgacttctgaagctgagtttataacttttcatcatatgtcaatctgacttgttggtagaagactttgtttttttttttttgaggcagggttgccctcttgcccaggctgaagtgcagtggtgtgattttggctcactgcaacctccacctcccgggttcaagcaattctcatgcctcagcctcctgagtagctgggattacaggcatgcgccaccacacctggctcatttttgtatttttagtagagacagggttttaccatgttgcccagcctggtctcgaactcctggcctcaggtgatccatccgccttggcctcccaaagtgctgggattataggcatgagccaccatgcctggccggtagaagactgactgtgtctgttgaagagtttatttaagtttcaaaaccaaattttctcttttcttagaaatagcctcacagtctggcacttcatattaatacctccctgaaattaatttttcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaaggtaagtggtgaaataaaattcatttcccacgtctctttaccagttataaaagacaataggctcaaagaagaattgagtacaacaaagggcttgctctaaaggctgtttgccaagaggaatacacacaattcttctctcctgaggctttctctgagaaataagactcattgattctggagcttgggccgtgttacctcttttttgcccagttagtttgggtctgatctttgtttccaaggtaaatctgtgttcactgttggccattgagacttataaaaagtcttcctatgtttgagaagaaaacctaaaattcttgaaatcgaggaagatttgggggtgaattatggagaaatttctgtggagagataagttatctacagcagagtaggagattttcccaagaatgcataggaaagcattttttgccaagggctctggagttttttgcacataggaacctttttttcttactagtatttcataaaaaacaattcccatactcatgtgcaaataaagacattgcttcagactcttttcaggacaatgtttctttcctttgcttgtttggtctgagatcttggatgatatgctgtatctttctaggatgtgcagtttgggattgatattatgaaggctgacttaacatccatatagtataaaataaatgtcacacatattctgcatttataatgagttatgcattcttttgtgtttcaaaaatcttacactatcttatcttttctgtgaaaacctaacttaactaatgagatccctatgatataaatttaaggaatgtaagggctgcatcatagtttggttggatgtaccaaatatttttcttttcagtgaagataaacagacattttatgtatttacgtatatgcctttttacatcccagagtatttgagacaggtgaagatgacttagacttttttcccagaagcagcttttacagggcaagaatttcatcagctttgggaaacacacttgcatatctctgcttacatttcagtagtgtaatatggtcagtgcaatgaaaaagtggagaccacatcaaaataacctatgccactggattcacaatgtttgagaaatatctttgcccagagtaagcactgtcaaagatagaattctgtgccctcctccttccctccacaagatttgaaagagacaaggctcacatcttggagaatttctggctccttttgacctggcagtcttgagagatgcagctcggtcagaagattgcaaggatttcctgctttcagcctgtctagaaatactacaagatgaacatcccccatatctcattatttacttcttcctaagtcaggaaacttggagacatgtgaaaattcatttcatgagtttcagtaaatattttattttgagaggctgggtggtggtttgggtttcttttgtttatttcctttttttgagataccgaaatagaattgatttactaaataggtttagtcttacgtcaaagggttaatttagcttccaaaggcttgctctgtaagcaagttatgtaatatttcataacatgtggatgaaaggtaggcaatattaagaagtggcaatccctagcactgtttattggtacactgcctgtctttgggtataccattaaattctgcttcctgtctaagcttaaagttctaggagttgggctgtccaagattttggccatgaagttaaacaatgggaaaggaaacactgaagtattctctatggataggtgtttaatgtcccctctggtcgccaccttacttccctagtcttctgaccccattctcttcagcaatggatggagccaggaagtgagccctggcctcataagataatggctatggcatgtggtgggctagattggctgcttttctgtgctttccagctgggaaggaaatcaaacttctgctgttgcagggaattagctgcctttgtcccctgtggtttaattaactctttcttcactttgactgactattatgaagcactctgagaatgcttgatgggatgtgttgggcatagcaatgtgaaatgttatctctctgagatttcaagcatgactccacaccacatcatctctatctctgaggaatggactaggtttccagcagcatgttaacattgtatgagtaatgtttgattggccttgaaatctttttttttttttttttttgagacggagttttgctcttgttgcccaggctaaagtgcagtggtgctatctcagctcactgcaacttctgccccccggttcaaatgattctcctgcctcagcctctgaaatagctgggactacaggtgcgtgccatcatgcctggctaattttttgtatttttcgtagagatggggttttgccacgttggtcaggctggtctcaaactcctgacctcaagtgatccacctgcctcagcctcccaaagtgctgggattacaggcgtgagccaagaacccagtcagaatctcttcagttttcttctcagtctttggagtggtgacttttcaaatgtttgtcattgaagatatcaatgactgctaaatgttaaactaaatgcaaaaacaattaaacatggttttagaaagaatcatatccctagtcttcagaatcttaaaatgctcacatgaatggtcctcttgaataaccaaattcaaaagtgttagctgtttcctgttaatctaaagatcctttgggatccattcatttattttcatggaatttacattatttacctaaagagagagcacatgagtattttaaatattagtaaaacttgtcggtaaagtgtatagatttaactttaaattttaaagtaaatattatccttcattttgaaaaaattataatgattaatcttttaaaatgtgaaatctataaaaatatattctgcttgtcaataaaccttgtgaaaggagtcaatctcaattgggagttttttttcaaaatttttatacacacagatatatacacatgcatgtgcatgcacaaacacacacacacacatacacacacaccctcatgtagcacagatatctatcagcagaataatctgtggatgcctttggttgtgtgaggtgtcccttccagtcattcacttgtctggttagagtttaggaacctgaaaaatgaccaacttttctagtaaatactattaactcattaataaaactaaattttcttctagattacagatctccatttattgcttctgttagtgaccaacatggagtcgtgtacattactgagaacaaaaacaaaactgtggtgattccatgtctcgggtccatttcaaatctcaacgtgtcactttgtgcagtaagttgcatctcctccaatcgtctcttaagtttttataattttaagctaatattaagatgggtaacctgtttataatattcacaatgagttttaaggatcctttaggaagggtcaaatgcaatgaataaaactaattagtattcttaaaaataagatgaattcttcagtgatcattgtacatggctctcatttttggtactggattaaatatttgatatgtctttttattacccagagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtaggtaagaggacatttcctttccatatcattaataacatatccttgtattaagatcttggagataacaacatagagtgaagaaggatattgaaaagtataggaactcaggatatggtgttgggcaattcatctgctcttctctaccaaataaacccatgtgcaattgaggttgtctcttttcttgccaagattaaggaagaaaaagaaaactttttaaaaaaaggatgaaagcgaatggtattactcgagcacattttatgaagaattcaatgttcagagcattgcttgctatcaattatttcaattatgactattttatggaaacttcagcaatttgctaaagctggccctactggcctagggctactgaccactgaaagtttactacttttctgtccactgggttacaacatctttgagatctgtgaaggtagtgctttgtaaacctctgttggccattttcctgggagctaccaagtattggtgaggcctgcagggaaaaacaatgtggcatgttttaaagttgcattactttaaaaaataaatctgtgcaaagttataggcttatttgctctctcatgttctgttttttcaatttacttgctctagggtataggatttatgatgtggttctgagtccgtctcatggaattgaactatctgttggagaaaagcttgtcttaaattgtacagcaagaactgaactaaatgtggggattgacttcaactgggaatacccttcttcgaaggtaacgctaatgattcaaagccagacctccaaatacttagataataagccccagtgaagtttgcttgagagataggggcctctttggccagataaaatgtaagagccttaaacacacacacatacacacccactcacacacacatacacacacacacaatttaagggaattgcagaacagatagcacccaccaaaaggtgaaataccaggaattttgtcctattctgcaatagccaggctatgaatattagttttctctaggtgattacatctttccacattatgtcatttctctgttctccaaagtttttgatctacattccttttaagggaatttctctttaagaggtggcatgagatacactgctccttaaacagtggtcacatttacttgtgtttctgcagtttatatccatctcactttcaccacgtgaggttttaaaaatcctaattcagttggttccatttatttctcctgaaacaaaatatatttgttgtctgcatgaggttaaaagttctggtgtccctgtttttagcattaaataatgtttaccaaagcccagatttaattctgtgtgttactagaagttattgggtaatgttatatgctgtgctttggaagttcagtcaactctttttttcagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatggtaagctatggtcttggaaattattctgtgccttgacaagtgagataatttaaataaatttaggtcacttagtgattcctattttgttcattcagaagatagtttctagtttttcttgttagggaggccacatgacctagaggtcaagagcatagctttgtagtcaggaacttgggttcaaacctcaactttaaagatgagatgtgctgatatacagtaagagttcatttagtattacttattatagttattgctgctattaggattgttactatgataaatagtattagctaaggtagtttttaaattttcattttattgcaaggctgagaggcctacttgaataagcatgagctttgcaaactggggaaacatttagcaatatacagttgacctgtgagcaactcagggattgggggaactcaggggagttcccctaactttccctcctctgcagtcaaaaatccatgtataggccgggcgcggtggctcacgcctgtaatcccaacactttgggagtctgaggtgggtggatcacctgagatcaggagttcgaaaccagcctggtcaacatggtggaaccccatctctactaaaaatccaaaaaattagcctggtgtggtggtgggagcttgtaatcccagctactcaggaggctgaggcaggagaattgcttgaacccaggaggtggaggttgcagtgagccaagatcgtgccattgtaccccagcctgggcaacaagagtgaaactccttctcaaaaaaaaaaaaaaaaaaaaaatcaaggtataacttttgacttccacaaaacataactaatggcctactgttgactggaagccctactgataacataaacagtcaattaacacatattttatatgttatatgtattatatactgtattcttccaataaagctagagaaaagaaaatgttattaagaaaattgtaaggaagagaaaatatatttactattcattaagtgtaagtggatcatcataaaggtcttcatccttgtcttcacgttgagtaggctgaggaaaagggggaagaggagggggtggttttgctgtctcaggggtggcagaggtggaagaaaatctgcttataagtggactcatgtagttcaagtttgtgttatttaagggtcaactgtaattgaactggaattaaattgaactggccttgagaaaatcaccttaattttttgtttattctctttcatttacataaatgtctgagtttacatggtaatttgtgtggcatcctacttataagccttggaaaggattttggagtttatattatgagaatgcatcaatacagtgaaattttaaaaataccttagataatgctatttattagagttgtaatcataaaagtggcaacaactataacaagtatgatttagtgagcacttactttattagctcatctcatctttgaagctgagattggaactcaagttcctgactacaaagctatgctcttgacctctaggtcacgtggcatccctagcaagaacttgaaaatttcttctgaatgaacaaaatagaaatcactaagtgtcctaaatttatttaaattatttcacttgccaagatgcacttgtcaaaatacacagagagagatgtgctctggcttatgtttttatagaattacttttgttttccagaatacttcagggaaataggggcagaaataaggaggtcagttgggaggctaattgcagttatccaagtgagagttgaggggtggcttagacaagggtagttgaggtggaggtagtgagaggtgatctgcttctggatatattttgaaggtagagtcaacagggtccgctgatcaattcattggttgtggagtataagagaaaaagagtggaagatgactcgagcgttagcatgagcaactgagtaaatgatggtgttatttactgagatggcaaagatcgagaaggcagtgagatttagggaaacagtgttagatatgtttatctggagatgcctgttaaacatccaagtggagatatttaacatatcaacccggaacccagaggagtcagggcagaagataacacatttaggaggtacgtgaatgatactttaaacctgaggctagaggaaggtgtaaataaagaggaggtctgaggactgagtcctggggcctcatggtggaagaggtgtgtggaggctgtcatgggagcagaggagaaggagcacccaagcatccctgggggacttagagaaagctgcacagaggagcaagtgtttgagttgagacttgagcaatcactaggcttgtgggagtgcactagcggggagagaaaagcaaatgcaaacacaggaggtgtgggagaaacacgggaggtgtgggagaagctgaaaagtgacccactgaaagatagtacaggaaatcttggaactgcagctactcagaccctcaaggtctttgacgtttcacttgaaatgaaaaactaaatcaaatgaccatttacagtaagttgacctttttttttttttattttcttccagaaaaaccttttgttgcttttggaagtggcatggaatctctggtggaagccacggtgggggagcgtgtcagaatccctgcgaagtaccttggttacccacccccagaaataaaatggtaactactggaaataaatgcaaagcatcatttcgtgtgagagcaaatcctttgactatactaattcctgagaattttttttcataggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatggtgagtccattcaattttcctctctgcccaagatttattatgatacattgtcttccaaatcagccaaaccaccgttcctctgcctcctgctgcttcactcatatcatggctgggcctgcgtacaaaagtcatctggcgtggtgaagctgaagtgaaacgtaggaccatgtgctctggccatgtttgtttaagaggccgtgtaaatgagctttgtggtggacaaatgcaagattaaagtagtgataccctcgatagctaaatgttgtgaaataagaatgcccacagggacagttgtcaagctaagttatactaccatgttcccctctcatggaattgcccacctggtacacagatgtgtaagacccttctccttagattttgtgcaaagcttctagtttgatgttgtagttgatgtatcagagatgtgcaggcacgttccaactctgaaggcttttgaagttgacactgttggcttggttgggagcttttcttttttcctttttgacaggagttcaggatctgattttgagtctgtaaaggaaagatagtaagtttttgatgtaaagataatttgaactttgttttctgaaactgaaaggtacaaataagtgtttggaatggagtggggagaagggtgccatggtcaagtgagtgtgagaggtgctaaggtgatgtgtagatgtgtaacaggtttctttattgcaggacttcgcagaaccttttatatgctaatgtatattggtattctccaggaggagagacatagagtattcaaggtttaacaaacctatttgaccagagcaccttttttcccctgagcaaattcattaatctctcactccaaacagtttgagaaatgcttctctgttgtaattctttgttcccccttctggtacggcatattaaaacttcaggatattttcccatgacattaaggtgcttccctacgtgtcctgatactcttctgtaggccgctgaacttggctttattattttttttcagggaatattttaaagataggctgggtgccgtggtttgcatctgtaatcccagcactttgggaggccgaggcggatggatcacctgaggtcaggagttcgagaccagcctggccaacatgatgaaaacccgtctctactaaaaatataaaaattagccaggcatggtggtgggcacctgtaatcccagctacttgggaggctgaggcaggagaatcacttgaacccaggaggtggaggttgcagatagccgagatcgcaccattgtactccagcctggtgacaagagcaaaactccgtctcaaaaaaaaagttaacaggttccaaaaaggttgtttagaagcagcataggtgtaggggactggggagaggagaaactggaaagtgtataagtaggatgggaggaggaaatgaacaggaaataaaaacaaaacacggacagcaaatagcccatttcatcagttcatgaagccactaaatattttattcactttagcaaattctctgctatatgaaataaacataaaaaagaagtcaagtcttcaaagcataatctgaggctttaggttgacagtaataaggaaatagttttgactttggagtcaaaaaagaaagaaaggaaaaagggagagaagaaagaaggaagtgagagaagggagaaggaagaaaggggaagagggaaagggagtggagagggagggagggaggaagagggagagagaatgaaaaactcagatgatggtggcaggaatgcattctctaaagatttacaccttcctttaacatgaggtggtttacgtgtttgggttcagaagtcagagtgtctaggtttgttccaggttttgccgttcgttaactgagtgaccttgggcgagtcatttttttctgtttcatttttttctcacgtataaagctgtggacagtaatagtggttgtgaggattaagtgaatgaattcatgcaaagcacttcaaacaatgcttggcacataataaatgtatttactgtgctatttcagctgttttctgtagcctttccctgatctcctaaacttgagaggacagagagaactatctctgtaatacagatgagaggcacaggatttcaacacttccataaagtcattcagcttgttagtttattattattattagcttattgtcatttttattttatttcgttactttattccttttttttttttttggtagagatggggtctcaccatgtggcccaggctggtcttgatctcctgggcttaagcgatccacctaccttggcgtcccaaaatactgagattacaggcataagcccccatgcctggctagttgttatttttatgagtatcactagaactcaggtctcttgtttccacatctaggtgttcttcgaaaaagaaagtggaagcaaaatcatatgcttaaagaaagtcagctttagttgctaaaatcctctatttcccattcttcaaagctgactgacaattcaaaagttgtttttcccatcttcagtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccaggtgagtaaggccacatgctctttgctttcctgccatcttgcatttcttacagctgagctatgatatgactccatcctaaatggagaagcctaaaccaaaaaaagttttctctcaagaggtagcctgaatctccatccatctttctctgtgtcttacattttaggggatgtctttgcttggagtatcctcctttggggttagctaagctcagccttgttaggttagccgtgaggtacacttctccaaacacaggctatttgctcagtttgctaattgccagtctttggtttttctcccgataccaatcggctggtgaataccacatccctccttcttgtgtgtgtgaagatccatctctcagaggaaatgctgatagatgagaggcagtgatagacccagccccagtcctcagggtctcaggcccagcttatcatgctctgacacaagtccagacatccttagggaaaaacacaacaacagcagccaacccaccaccaccctaagcagtccacttcctgttgttgtttttgaaatggccactatgagcttcttcctcagctgctgatcatttccttcacagagaccatggtcccagagaaattactttaaggagcccagtggcttctaagtttccttgccttcctttgaactaaattaacttgaattgtcttgtcgatccaatttatgaatgaaggtttattcccagaatagctgcttccctcctgtatcctgaatgaatctacctagaaccttttccttcattgtcaatgcctatttttaattggcgccaagtcttgtaccatggtaggctgcgttggaagttatttctaagaacagaataaccaaagtctgaatcttttccttactcttgactctaattaaagaaaaattaaatcataatatgcgctgttatctctttcttatagccaagctgtctcagtgacaaacccatacccttgtgaagaatggagaagtgtggaggacttccagggaggaaataaaattgaagttaataaaaatcaatttgctctaattgaaggaaaaaacaaagtgagtttgaagttttaaaatttgaaaatctctctctctttaatggaaggatggtacaataatatgtgaggcatattggagattaataatcaaatagtctggatgattaaatagagcgtattaagtcactttgaaaataccattgacttttagcagtaccattaacttattaatagcttatcagagaaaaataaaaacatctatgacattaaatctatgcatctgtgtagggtgattctgattttataaacatgagaatgaaaaaatgtgtatcatatcatattaaaacacatcattagtttcatggcttccaaagccctttttatataatgtgtgagctccacagcagcataattatacaaattgagtaaatatcccaaacctaaaaaccccaaatccaaaatgctccagattctgaacctttttgagtgccgacatggtgctcaaaggaaacgctcgttggagcattttggattttcagattagggatgctcaactggtaagtatacaatgcaaatattccaaaatccaaaaaaaaaaatccaaaatccaaaccacttttggtcccaagcgttttgagtaagggatactcaacctgcaattgcataaatttgagcgtgtccaaccgctgcagaagtgggaatggcataggcaggttggagtgattgtggagactgctggactgagtgcttgtgcacaaacagccgcgttgtttatggcctgggatttgttttttccccgcacagactgtaagtacccttgttatccaagcggcaaatgtgtcagctttgtacaaatgtgaagcggtcaacaaagtcgggagaggagagagggtgatctccttccacgtgaccagtaagtactcttctctggaggtttgggttggatcactcacacagtgggtactaagctatgtaattccctgttgtttttgccattcatgtgagtggcatggcatttaggaaagaggacttggattgatcattgatgctttcattcataaattacaacttctcaggtatctcctgggcttatgtgaagtcagtgcgtctaactacactggagagagaatggtttcacagatgctttaaaccacaagctctgtgtggtatttacatctcagtcttcagagtctggcacagtgcctggcttattgagcttcagtacatattggtgggcttgctgtggaacagttgatgagggtgggctttatggaggcaatcagaaggacataggagcagtgccctcccaatgctgccgattttgcctgtgcatcttagttttatggataagctttagctgattgtgctgaatggaatattatagccagggctaattcattggcataaatgtagctttcatatcattgagtgttagtgttaatgaagacctaattttaaaattctgttagaattagagattttgctttggatttttaatatattaaacattgcgtagagctcatagtggagatgtggtaaatatctgaggaattcgtttacattttcaagtaatgtgtttggccaaataagatattttgggacctgaattgtctagtttgtttgtcaagttgtagtacatcacctggaacggatagagcttcatttcttttggtactttgtagtagtctgaaagcagcaagatgatagtgagctgtaccaagttaaatcaccattcaataactatggcctcttcattttagggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctaggtagggagacaattctggatcattgtgcagaggcagttggaatgccttaaatgtagtgcaattcaggtgctatgcaaagattactgtcctctaggagattatgttgtaaactggtgcacacttcttcaccgaaagtccttgaggaagaaagaagctaataataatgaaatgatatatcgaaaggagaaaataacaaaacctgatgatggagtaattcactagtatatgcaagggatattagcttgaaccagggaaacttctgccttatcttgggcatccatttatttaaatagacaaatatttgtggaatgcctgctatgagctaggagagtgtcagaaattcacagtggtaaacatgaaggaaaggaggagaacataggcaaccactgggaagtcacagcacagtgaggtctctgtgtccatgagaacaggaattgttctctgttttgctccctgctatagctctagtcatagagcatagcagcatatactaactgctcaataaggcacctgctgcatgaagagtgggatgatgggctgcgtttaagacctagaagactccatgggaaggaagctacattcactgtctgtacctctgggtcatcccacatgatccagcgtagcccaaggtcaatgggacgatcacttcagtgagcagatagctctgtaaattcctccatagaggcactgtctaccccttgtctaacctcatgccttgtgcaaaagctgggcagccatggctttgtctgtgggaaaatcaggcaaatttggggagcgtctctttgtgccacttctctccattttctcctcttgtggtgtccctttccaattcctaggatatatgtgccctctgttttttttttactgttaggaaggaaattgcccaagtaaattcatctataccacagttttagagggtaacgtcttcatcagaggccttggcgtatttgaagaggcaccttctgacagacactagcataaagttcgctagttttaagactcaggtgtcataataagagatactttggggtcaagtcatccccagcatccttcaagtcacaccacatagatcacatggattttctgttggcttgtctggcttcaaggttatggcagaattgagaaagagatgtgaagtaggctcctggcctagctgtgcccagaaaatatgtgctcgcagttagctgctttgcttccctaaggactcctaacttgttttcctaaaacctattcttagaaataggctagaatccagtacatttgcttagacttcaatgtagtacgctgttgaggtaatctcattttgctaagtgttgacgtggattttttcagcatgattccttttgatgttcagttggttgggacaagatatttccacagcactttgatgatctgaagaaagaataaatctaaagtgttcttgtacacttaaacaaatactcatgggcttcattttctttaaatccaagacttcccttagggtattgttgttttgtttgtgttttagtggaaatagcactgaactggtcttttagcctcaccagattctgtaaacagttcaactgtttacttagttgcagggacatggacaagtggtttaatgtcgctgaacatcatttatttcatctgtgagataacgctaacagtcctattctgctcattacataagatcactagtgaggaacacaaattgtgtaaacaagttttataagaattgccaaataaatgtaaggcattattggttgaatgatactaaaatttggcacttccaagagaaatttgaagggattctagggtattattgactagaatcttcatgggagggaagttttcacctggggaggctgtgtctaattagaggaaaaatccataaaggtgaccctgaacctttcttttgtgatgggattaccagctagtatcactaatatgaatgttaaaagccattaatctgtttgcagtgtcctgactgacttgtttcatttaactttacccagtgaccagtgtattttcccagaagttaatatatcaacaagttcctttttactaaatttaaactgtttaaaagtttgctgataccagaaccatttcaaaagttataattccatgttctgtgattttctttttgtgtgtctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggtaaatagaatttggctatcactcttgggttgcagaactttcccagggatgttatctaaaaagccatattatttcttgatgtaatgtagaaaaaaagcagtattggtgtccatgacctggctcatttcacagacttagaattggagtatggggccctgttgaattttcatgaaagccatataggagattagtcagcagtagatcccatgtgactctacagagttagataatagaacaagatgaagggcagcatttatattttctaaatttccctgaaaaacttcacagactacatcatcataaatgagaatgatcgttttcttcctctgttaggcattgtattgaaggatgggaaccggaacctcactatccgcagagtgaggaaggaggacgaaggcctctacacctgccaggcatgcagtgttcttggctgtgcaaaagtggaggcatttttcataatagaaggtcagtgggataaaaaaaaatgtggtacatatacaccatggaatgctatgcagccgtaaaaaggaatctgatcatgtcctttgcagctgcatggatggagctggaagccattatcctcagcaaactaacacaggaacagaaaaccaaacgccacacattctcacttataagtgggagctgaacaatgtgaacacatagacacagggaagggaacaacacacactggggcctactgtgggttggggagaaggagagcatcaggaaaaatagctaatgcatgctgggcttaatacctaggagatggattaataggtgcagcaaatcaccatggcacatgtttacctgtgtaacaaacctgagcattctgcacatgtatcccggaacttaaaagaaaaaaagaaggtcagtgggaagtcatagatacatcctgtggtttttgaagattagtttgtatcttatagacacacattcactttgaatagggcaacgacagatgatttttaatattctttgtactttgtaaattttctcagtgagtatgtattcttttaaccagcaaacataattaatgttgttataattctgcttgcatcacatttcctattcctgcagttcttattgtggaaaaattcttaatcaggcaggatgaatagcctcttctccctgattctgtctttgtttgaatggcttgattaacttatagaaatgatgcctttatatttatttggaaaaacattagaattgctgcctaatcatggcagtcaatgctatccagatagtcacaaggattccgagttttaattggactagagataattaagattcacttgtgaacaatagaccattgctcttctgacatggaaaatttttggtttttatctcaatacgtgtgtatgcagaagtgatgtgaaatctgtcattttcttagctaggaaaagtaatttgtggcagaatattttatcttaagaagtatattcctatggcttttttttttatagcccaccagggaaagaataaaactgtgttgtggggtaaaagaatggtatgcaagggtaagaaagaagtatggtgatagaagggatcgatggatttctatgaactcatcctaacttgtctctcaaagtctagattttggtccctttactctgccaaatctatgatgccaagtattgcatcgagatatgttgacatattttcaaatgtataagcttattagcatttcataaactacacttgcaaataaagatttcaaagaccatggcggttttgtcatttccaaagtgattcatgttttagggcaaatccgcagaatgacgtctagattgtctctgatgctctgcattacctcttgttggtggcctgcagctggttacagatgcctaactaggtaacactggcacagagattatagttacttcttacctggagtgaatgctaagaaaggcagagctagatatttaatactcctgctgggttcccaaatgttatgcgagaatattaatatacaaacacatagaaaacagactctttgaactttttatcctctatgttcaactggacttttaaatctgtgtgtataaatagagaattacttccctaggaccaccagagaaacaaaatttactccaagcataattgtgcttgtctctcaatggttaagttaacttttattttgcaaaccaatttgttacttattttgcaaaccagtttcttacttgtcttcttctctcttgaggccgtagtgggccatccgcacagcttgtggcccggtttgattctccttgcactcttctgatgggaggccccaagtgatgactgcttccttatcatctctttgctaatcactcttagtggaaagcctgtttctgtattttgtttcttccactcagagctgtcctctgaagccctgagcatctgcagctttgcttgctgacttctagtttcctcttctctttcctttcatgagtgatttgaaactcccattaccaggccatgcgtgatgtgctcatcttggctcttcctcttctcctcactcagactcctgccacaagggatggggtagtgtatgtaatggttagttcatgttggacaggcctctttatctcttgactgaaccactgactagctgtgtgccctcagtcaagtagcttaagctctctggtcttctgtttcttcatctgaaaactgagagttgttgaggagattaagtggaatggcatatttaaagtgatgagtgcatagtagatacatggtcattagtaactctcaggtcaaaaaattttgtttatttccctacttggtttcttatgtgatccttttgcaaactctgcacagatcaaaatattgactatcagtttaaaagaagacttttgttttcctcaaatagaaatatttttttttctctgtagagaatgatctgttttctttccatcaaagactgctcttcctctaaacactttctatgtttggcttttaagacattactacttctatgcttaattacttaagaattttattgttgtaagtttacatgagcaatgttttgcaagctttaaattttccattaacaattctgtaggccaggtgtggtggcttatgcctgtaatccctgcactttgggaggccaaggcaggggggatggctagaggccaggagttcgagactagcctgggcaatgtagtgagaccctgtctctacagaaaataaaagaaaaattagctgggcttggtggtatgcacctgtagtcccagctactcgggaggctgaggggggagaatcgcttgagcctaggaattggaggctgcaataagctatgattgtgtcatggtactccagcctggaacatagaaagaaaccctgtctctaaaaataaataaataaataaataaataaataaataaataaataaataaattaaattcaaaaaaagaattctgtagactccattcaagttacgggtgtgtaactgttgtcctctaggatttttccaagttggtaagcttgggattttgctttagtgctaaaatttgtcatcttacaaacaaaaagtataagtttccaactgttgatactcattcaattgtgtctttccaggtgcccaggaaaagacgaacttggaaatcattattctagtaggcacggcggtgattgccatgttcttctggctacttcttgtcatcatcctacggaccgttaagcgggtaaaaaaataatttcccttctgcccatgcacattggttttcatgattaatgaaaactgactggggttctttgagttgtttcttcccattgttattggctcaatgggcacatttttatttcaatacaataacgttcctgcccactttcttttggctggatctcagggatttaattgatagaagccactagagaggaaaagggcttggactgtctagtgtaattaagctttaaaaccttaattctgagctcctttgggggacaagggaaactagaagcagggttataataggaccactctcaaactccatgagttttattggaaaatgagacaggaatgaggctccaataaacagcaataacaagcacacaaaacaacagccaaacaacagtgtgtttatgactggaaggattgatgctttccaggccaatggaggggaactgaagacaggctacttgtccatcgtcatggatccagatgaactcccattggatgaacattgtgaacgactgccttatgatgccagcaaatgggaattccccagagaccggctgaagctaggtgcattttcaattgctattaatttgatattgtgtttaccaggccatctcttcctccattagaatgatgacaaatgtggtgtattcagatgttggattctggtttagaaatattaattccatttcttgaatttgtataatcattcatatagccacttagaggtagggtccctatgtaatcatccaaagcaggacatttggagagtgaagggggagttattaaataattaagccaggacaaaggagtaaactggactatccatgttaaattgggatgtatggtcaccctatctagttgatgtctctgcgtatcactttggttgtatagtaatccaagtctgttttcttgttgctgttgttgttgactctaggtaagcctcttggccgtggtgcctttggccaagtgattgaagcagatgcctttggaattgacaagacagcaacttgcaggacagtagcagtcaaaatgttgaaaggtaaaagcaaaattatgtggtgatctatctttctgttttatctagtctttaaatatgttgcaaggcttgtatcagtagctttgtgcttatgtgggcctactagccacacatgcagtcagcctaaataatgcccttgtgcaaattggaaaaaggatcctcctttgtagctttatgccaggatgcatggtctggcaagcaaagttgggaatggctttcaccttcttgcctggttaccctcgtgcagggctcagccaacacagttgtacttagtggttctgggtacagggaaaaaggactgtggttatattaaaattgtttcttaatatattgtggaatcagataattatagaccatctagagacatggaaaggaagatagtgaaatacaaaaatagcatgttctccagaattggaatatgtaaaagatgttcatatgtaaaagataatttgcaaaacaagaatggttgtgttagaaaaaaatataatgggttatattttttaaattaaaagctttataaataattgttaattctaatagtaacggaattctggtctggccattttcattttaggaggttagacagtaaagcttctttcttcaattgtgatgttctttcattgatgaaggcagtgccaatgaccctttgccaataggttttgtgcatttcaaagctatctttctccatctgccttttttctcttgtggccaagggagtgtgtaattttgaggtggctcatcagagccttagatgtggaccatgcctgtgaattagtgggaagtgtagcagtccatacaggatcaaacacatagtcttagtgccatcagcctcatgtgccaactggtctttccagctggccttaattcgcctgcacagatcggcacagattggctggaacattcggtatagcccctaacacgtgaagatatttaatacatggtgttgcttccttatgaggaagtgctgaaatgatcagaccctcagaatcatagtgaacctgaaatgcaaaaatccagttttgcagaagaagagaatctgggcatgattccactgcagatgtattctccgctttgcaaaaggtttcacaatgggttcctttaaatatcaaactttctggctcacttaaaatatgaattttatttcaaattagaaaatagaatttacacttcacttttgaggaaatgcatgtggtctgtaaactaggtcacagctgtgttaccccggagggtaagttgtatagtggcatgcagggagggagggaccccaattattgaaggaaatgtccatacctatgatttccctctttgtactgtatttgtagaaggagcaacacacagtgagcatcgagctctcatgtctgaactcaagatcctcattcatattggtcaccatctcaatgtggtcaaccttctaggtgcctgtaccaagccaggaggtgagtaactgtgggtggttttggtcacccaattttaacatgcctctctgatagtgtttgagggaaagcagtcaactcctctggccttgattttcttagcttagaatactttgcggattcctaggaataaatatatttcatggaggtttaattggcactagaattaaattattgtaaaactttctctgaattaagaaatgtcatgctactatgatacagtttgttacttgtgtaacagatgtccagagaagagtaaacttccctaaaacttgaaagcttaagggtagttacccccaaaatggaatcatatcaggagattgcactgaaaagcaagtagatgggtgggttttcttctgaaattttggttaatcttgtgaaaatgtgttctggaaaaaagaaaagctacaatataaggggattgggaccagctgatttctacactcctgtcccaatgaaaggttgtagccttcttctaaggtgtttttgggttcatcactatattaaacgcttagtgaggaatatgagtgaaaacccattttccttcctggacatgctgcctgcagggccactcatggtgattgtggaattctgcaaatttggaaacctgtccacttacctgaggagcaagagaaatgaatttgtcccctacaaggtatgtcatctcctaatcctgctctggccatgttataaaatgaagggaaactcaaaatggtacaggttagttttttagttgaaattttgtgaagaacttgtgaggaatcttctcatattacctcttggctgttgtaacttcctcttttaccttctgggggccatatgtttctgttttatgtatgtgattttaatctactgacccattacagagtgtggacatgggggagaaggcaggtatgagcgaggaaaggggagggcagagggtaggacatctctgggttattctgtctctcccctagccatatttggccccgtggagtgtaaatccctctgtgaagagcatcctaatgctgaaagtgtgtctgaatgcaactcaaaatgtggcatttgtcactttaagctaaagaaggagctaggctttgtggaagaaaccctattatgcacaaaacttgccccaagtttcagctcagagattgcataatcctgaaattgatgtcctccttgtctgctttttagtagtttcaattatctccatggtttactacattttaaaggttgtaaacttttaaagactcattttgtattcaaggagtttgtttgttcctttgcttttttatagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaaggtactggctagtgcttcctgcatgctatggcatgctcttgtcagagcagacagggtgatagggtgttacaaggaatttgatcatgggaaaagtccaatactacctcataatttgaaagagacctgaatttctataatagactgcctccattctgtctccccaaaagtgaagtgtggaagccctagactgggaagtgaagcagggctagcctgagaaatctgggtagtccaagtgggctaagcagtcggctacaaccacagcagtgttcttaaaatactggttcagcatttattagtgagagaggccacaagttttctggtagttgactagcctctccattgccttggagagccccagagtggtttgccccacgttgcatgctttacctgtgcaaaagtcttttcattatacctaaccttctcaaaggcagtttaggagccatctgttgtttctaccctaccccaagcggcttatcaagtcttccttccaaccatacttcctcaggcgagtcttgataaatatcctggcctttattaagttatgtttccagtgatattttatttatttgtttttatgtttatttttatttttttgaggtggagtctcatgctgttgcccaggctggagtgcaatggtgcgatctcggctcactgaaaccttcgccttttgggttcaagtgattcttgtgcctcagccttccgagtagctgggattacaggtgccttccaccatgcccagctaattttttttttttttgtatttttagtaaagatggggtttcaccatgttggccaggctggtctcgaactcctgatctcaggtgatccgcctgcctcagcctcccaaagtgctgggattataggcgtaagcctccgtgcctggcctgagtgatattttagtgctctttttgggtggagctgtggtcccagcctaacttccaggacttcagccggctccaggacacactgtatttctgcctccttcagaaggagcagagatagcgttgtggatgtagagatgggtgacaggctggctccccttgaggcataagtctagaagaatagtggaagaaacccactctgtttcccttgacatgaggctacagagagaatttgcatttaactccttttccttagaagctgagaaggtagtgtgaggctgggacttggtctagaagcacatggggaggtggtctaggcttcatttagctgggcccacactgagtggtgctgcctctaccctgctctttgtctttcaaaaaacagtggccagtgagccagaaacctaagagattgagttgttgagaaaaaggctcacagccttttaaatacttacgaatttattactacaactaagtttttgtttactctggtatttgtctccaggaaagaagccataagtcttatctgaccaaagagatgattttgaaacacccatttaatatcttagtgtttatttgtaccagttgcactgaagtaaataccaccaatttacgtaaatttatctttccatgtttctgttatctctcaggaaaaaacaccctcccaggccagatttaatgtatttacagcactttttaagtttgaaaatgaattaaatatatttctagtatttttagttatctattgcagattatagtttgacttttggcctttgtcccaggacaaaacctggagagaagagattcaatgaccctgaatattgttgttttatttttagagttcttgatatgaaactattgtttatccctctgggtacatgacaaaaaacagtgtaagtggcaaatttggaaatgtcctctttatttcccagattatctaggtcagtgttaccttattctacctcctggatttactggttcaatttggctaaaatggaaaaaccagtattgttcctaagggggtatgatgaaggctaatgatactgggattcaggagatttacagaagatagaagcattgactctctgcttctatttcctaaaaacttaactcccaagtcttaaaaagattattactctagcaaacttagaaacatcacactaactcatggaaatactgatctccatcctcctgcctctttggacagctcctgaagatctgtataaggacttcctgaccttggagcatctcatctgttacagcttccaagtggctaagggcatggagttcttggcatcgcgaaaggtaagaaaggttgaggggaaatcagctatcttttcagatcacaggtttggaaataagatgtccagtgtcagccattggtgcttgtttgggattgtaattcattcaccacttctacgtcttttagaagagctctactggggaggctctgtttctgctgagtaagagtggttaaggagttcatgaaattaagctgtataataaaggcttgtcaagcatctactaagtgtgaggcagtcttctgagcactgaggatactgtggtgaacaatcaggcaaagctcttcaccttcatggagtttacagttctagtgggtagagcaaacaataagcaatataaacaagtaaaacgtgttgtaggttagatgagagtaaatgctatggggaaataaagcaagaaagggttatagaatacacaggagcaatgcacttgtgtatgtttatgcttctctgtgtgtgtacatctactttaaacaaggtagacgaggaaggctttactaagaacttgacatttgagcaatgacctggaaaggggaggggctgagccttacagatatcttggcatgagaatcatttttaatttattttacattcatcaacatccatcaaaaagtatttgttaggagtataattagaaacgaggaaggacaggcttcagatgagagcgattaaaagagctaaaattagaaaagtaggccaaacaaaggctgagatggggacgtgacaagttacaactattccaaaggttgtaaacaccaagcggggagcaaggctggtggcagtgattcccctggaaaggataaaaggtgtaattttatattaggtaacaatacttcaaattaaggatcaggaagaactatcagttgacagaatgtattcatgcagcttaatgaagaaagaaagacttaagtcatatttttttttgtttttcctaaattagaatgaaatcttcaacccatgttttccccttctcatagcattaaaggcctcaggctctttgatgtttctgctaggtagctcttatgttctctctcccaaggggaaggaggagaactgggaccttatagggttttcccaaagagaaaggccctttacacttcttggagattatgacttattattaccatttttttatggccggaattcgccacttagtcagggttccttttggggactaggaagagaatggaaatgaatgtgggaatgctttaactttccttacatctaccagactatttcttgaatccacttggttgtcgggttaaaaaaggaaactttttgtttggggggaaaagtcaaaaacactgtctgttttttggaattgccagtgttgctcaattgtgctagataatgtgcttctgaatatgccttgttcagaggagagtgccatacagatttgaggtgtgggaaggtcagcaatgcctggcttacatgatcacttctccaatgatttaagaattctccttttggccaggtgtgttggctcatgcctgtaattccagcactttgggaggccaaggtgtgtggatcacctgaggtcaggagtttgagaccagcctggccaccatggtgaaaccccgtctctactaaaaatataataattagctgggcgtggtggcacacctgtggtcccaactacttgggaggcagaggcaggagaatcacttgaacctgggaggtgaaggttgcagtgaactgagattgcaccactgcactccagcctgggcgagagtgagattccttctcaaaaaaaaaaaaaaaaaaaaaaaaagttttcttctaagccattgattcatttcttgtgctccccaagactcattttcttacaaaatatcatgtggagctaaagctgccgagtagtaggaagttagctgaagtttggaggatacagagaaaggagaaactgagaagctaaaaggaagagaaagaagtcaagatgaatctcattgtactattaatgcactagaaaatcaacctgacttgtgataggctgaaattgccttaatagacctttataataacccagcactttgaaatcaggggaagccacattgggaattgtttatcagagccagtctggcttcagcttcatacggaagggggaaaccaacaaagagcactaaaccaatgagagccccttgtttctgatttccgtgcattcattcaaaaaacaaatcccgttctcggacctccttagaataacacgttttaaaccaaatatggggccaggtaaaaggaatgtgtggatgtgaccagaaacacactcttttgtgtcctagaggagcctatttatgattccatcatcatattataacttaattatttaactccaaaggctggggctgtttatggaataagcagatgtgtgtctcagcaaagctcacagacttttttcctgaagtgttgataaaagatactaacccagtccttgttaatcagttggctttctgatgtgggatttttttttgatgcatgaggtcacaacagatgtgaaagagatcagctgtgccgagacctaatgcacacatgattctctttgcagtgtatccacagggacctggcggcacgaaatatcctcttatcggagaagaacgtggttaaaatctgtgactttggcttggcccgggatatttataaagatccagattatgtcagaaaaggagatgtaagtttcaaatatgaacccagtgcttggttaagtaacagaattaaaactcctcgtagagagcttcaggacctgtgttcaggaacagaggaagtttttttcttcagatatttgctaatttgggttctgaatccttgtcttctacccctgtaggctcgcctccctttgaaatggatggccccagaaacaatttttgacagagtgtacacaatccagagtgacgtctggtcttttggtgttttgctgtgggaaatattttccttaggtaagtcatttctttttgtccttccatccagactccaaagaggaagacaaaagttgtcttttcctctcctgtacttcatgtctatcaggcaaaacttctcggaagctttgaaaaaaaaaatagatacataggtgatgaggatgtgcaagattcaggctcagggttttctataagagaaaatcaaatcaaagaatgtctcctccctgttttattctaggtgcttctccatatcctggggtaaagattgatgaagaattttgtaggcgattgaaagaaggaactagaatgagggcccctgattatactacaccagaaatgtaagactttaagaagtattcctgtgttctctttctttgctcgcaaattctccttgcctggaagactttccattatatagaccttcttcattgcccagttagtgtcctgcttttactttggggcctttcttgataatttcaagcatggagtcatcacttcttgaaaagatagtactttattattcaaagcaaccagttagtttttattagatgttgctttaaatgttttctatacacattgagcctctggagtatgggactctgtgtcttacacagttttgtatccttatttagcatctcacctcgtcagctctttacaaatgtgtactcatttaagtgcttattttcagcattcaggaagaaagaggcatttaatgaaatcagtgttttgcttctctaggtaccagaccatgctggactgctggcacggggagcccagtcagagacccacgttttcagagttggtggaacatttgggaaatctcttgcaagctaatgctcagcaggtttgtcacctccatccaagaagcacctacaaagagtacttagatgtcaaggactttcctactgcctgaactgtctcatggctaccatgccatcctctcagccattgaataatctactgtattcttctacatctgagtaataatgcttttctaaaagctgtaattacccttttagacagataggattctaatttataacccgggagcagaccactctgatttctacctacttatctttttgttatattttcaaatcctcttctaaagttaaaacaaagaaaaaatctggttgatccacagaagatcaacaatggaagaaatttcaagaaatttttaataaattctgcaggcaaaaatacatctaagctatgcaaaagagatggtttctgtcttggtatcatcccaggttcttataacttccactggaagattttagagttgtagtgtttactattagaatgttatttaatctctagtcaatgcctcttactacaatggaagtgaatttcctctttcttttcttttgaacagctgggggacgataggtcagctctatttttatcaataaaccttccaaacatttacagatatcaaatagccctttatttctttttcttgatgcaataatattaagttgtgcaaccttttctcaaaagacccattttcctacccatttgttgcttttctttagactgtcatcagtttttccattgccttgaaatgtggtggctaaaactggatgccatgccctttgaagggcttggctcgtgtggttagggctttgtgaatgagtgattttttgttctatgtagctccttgtgttctgttgttacctctctgaccacagcctgctttctcttcattgtaactgcacttccctgtgggctgcttacccatcttgtttttagttctctcctttaatataccttccatttcaacagctttttgtttctgacacatgatttgtattgttgtcttaaagttctatgttcagatatgaaagccacacaccctatgtagccaagaagtccctgtgccctttgtttttaatgaaaaggcacttgaagaactgaagccataacaacagtcttctgtgtttattgtttcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcaggtactgtatatggcctaacatcccccgggggagggtgacttcaaggccatctcgggagggggattggaagtggaaggaagaccttgtctaaggctgttgcatcccacttccacataaccttagccctgaggttaacataatggggaatgctcctggaagagggcctgggtaggtgtgcttcctcccatctgtagcccacgctgctgccacagcattgcctttaagaattccaagccctgcagctgcaatagctggaatgccacagtttgctaatttccagaataaagagacgagttttacaaagacatctgcatttaaattatccccgtgtatgcttttattaatgtgaattaaatggcttaggagagattcagaaaggaagagttctgtgcttgcatgagaacatgcttatggctctctggcaaggatacagaaagccatgggtctgtgtccggaattagactggacactgcatctcagaagcccctcccacgtctgattttcagcattttatttgcataatgggatgtctgggcttatttaaaacacatgcactgcagtcctttcctgatttgcagaggggttctaaaggcagctttcttttttctctctcccagcacctgtgcataaggaaagagttggtgtggttttctacaatatgatattaaaattgccctttactaaggctgggactacttcattttgctttgtttctttcctaacccgtttgggtgttttcctgctttaatggaacccctgacagcatgggtccagcctgccagcccgagtgtgcctgggctgcagggaggggcagggagctctctcatgtccagaacttggccaggttgccacatggcaggggatgctaaggagaaactcgtggacagtttgccctctagagtcgtgtggggcagcagaaacactgatgggaaggaagaaagcttagaagccagcaagacagctgaccgttccattgaagtcaaaagcattaggcatatttttaaagaactttgccgtatattatcagatgttgcccacatcatgacactcagagtcaggcaaggtagaaacaatgatctttttttttgatgtattattgaacatgaggctcagttctattacctgagggcagtacaaacttgtagttaaagatcaggtattagagtcagatagaaatgagtaggacccccaagtctgtcttgtagcagctgtgcaacttggggcaaatcatctaccctctgcctcagtttctttatctgtgaaatgagacaaggtcagtggtgctgtttgaaaatggctgttttgagagttataagatataatctatttctaagcacctggcccttgaaagcactcagtaaaagatacctattaagtgagctgcttaaaatcacatccttgagatgaatccagttcctctgacccctaagtccatgttgtttcctcccatgccaaggagggccctcagagagaaacagtaatgagatgagactacaattccactcctgtgtttacacatttccagttcaagttgagctggccttttagtgtgacagttgttcccacacaccattattgcctccccctttatcagaaagccatttgatcatgaactacattccatgtgttttctgtgaccaagtagagtgatgatccgagtcggcagcctcctggctcaccgggtgctttgcatatggtgctgagcaggagaagaaatcatgtttgtgtaatggaagcaccaaatacgatgttggatatatagaagggctgctaacgtttatccccagaagcgtggacaaatgtgacaccacactcccagcacaggcctggctcctattttctgtctgtgatttttgaattggtttttccagcccagtttctcttttatccagccataatttgaaaaataaaatggaaattggaatcttttgtctgcatctcctctccacctcctccaccttttttcctttctataaaataaaactcacggtcacattttaatcatctggttttgaagaaaagcagatagaggcatttgcacacggcatgcttcattctgttgctctcctggggttctgtttctctggggagaatgagttgaggctggggtacttctcagggagcttgttctatcctcttacgcatttctggccaagtacaaaagctgagcagtctttctccttctaattttcaattctattgcattataaatagagttggacagagatatcactgtgggagctagcttcatgatttgttgcccctttaaaccatttgaaaaatatttacttagcatttatttagagaaaaggctgagaagtgtgtgggggagggaccactcatgtctagacttagctttgcctctaatttcccctgtggaccagctctggcctcaagtttgcatgcttcctgcaagaaaacacatacttgctgggctcatctttctttgagggcagtttggggaccatcggcaattgctctgtcattttccctgggagtttcacctcacacatcaagcagcttatcaaaaatttctttgcagttctctcttagagaaaggttttggtacataccattttcttcattttgtaattgttagggatgattaaatggcccttgtagattgatgcttggggcagcctgctagctaggtattcctgagtttggctctaccattagactgtttgcagtgggactgtcctttctgcactttttgtctgtttcataccccgtacttacacccctgaccctgctactgcatgatcagtgcatgcatgacaagagaacagtgctgtgcacatactgggtgcttaataatggcttgaacaattgtgtctgctgttttcttctttcttttccctcctgatactcttccaagggagtctgtatggagtagagtaaaacaaaacaaaaacttcacatgggctttagtgtctgaaggcctaagtttgagtcccagttctaccttttattagccattttctccctaatccttgactccctcatctccaaaggggaaatagttaaaagacctgtttctccgtcttaggagaaacagatgcaccattgtctgtgaaaatgctttgtcaatcatgagaggatcatgccatttaaaaaattactggattaagaatttaaggagctgtcctttctaaggcagctgaattattgtccaaactcgccaaccctagttgattctatcccctagatatctctagaatgagcccatgtctccaaacctcatgggcattccctttttctagccaagctgcctttctttctcctgaagaagtgcagtatttgtctcttgggtcttatgcctctagtcttattcttttcaatccagagtcaattctctaaagggcatatctgatcttgtcaatcccatgcctaaaatccttcagtggctcttcattgccctcaaaataataatccaaacattccagttatgtgattttggataagttcctcaaattttctatgccttggtttcctcatctgaagagttgggatagtaatactcacccctagagaggtaccgtggtgaacacatcatgagatgctgcttagacagcttctggcacagtgtcaggcttgcggcagattatcagtgagggcttcctgaacaagtgaatgcaggaatgattgactacggtaccagtagtgtttgacaactgttacttttaggggttggacttagaaagtaggctttgcttgcaccctgtgtatcatatcctcttaacttgtggagtttcctgagtgaggatgtcaccggaaaatctcattctctcctctctctatagggaggaaccagcctcttggggtaggggagagagaattaatttccattcttctcctttggcccaaggtctatgcagcatgttccagaagtctgcttgtagtgggaagtaggctggtataggaatgaagaatgtattttctgtctcggtgggcccttccagtgaataggacttcccttccctccacttgggctgtaagtgattttgatagcatcaactagactcacccaaagccacacggccgggaaggagcattctcaagaaggagaggatctgttgttcaacaagtcttattctttggactcctgaaggaagctttggaagtcaaaggagaaaaatgagctttgtttgaagagggcattattcttcctaagagcaataagcccaacattctctatgtcattcatcttcccaacatccctgtgagctggggagggagtgctactgccaacacatcttatagatgggacaagagggtcacagaaatattcatgactttctcaagtttctgcagtcagtggtagactctgaaataggcaaaatatcttgttattctcaaaccactgctctttcctgagacagcaactctgggggcgaaaacgaggggacagtgagactcagcccaccttctctttgcacaccaagcctctgttacatggaggaggaagaggttgtcttcaaatcactgctgggttcagtatcctttaaggagaccttcagatgtttcctctgcctatctttcattgaatggttgctctgtgagcattatccagaaaaactttcccaggagatggccagacagatgtgaaacactcagtaatatatccagagctcgatggaggaatcccatgcaatcaggaagccaagtagaaggcagttgatcactccatctgctgttgttgtctttagtccagaactggacctcagaagtaggattcaaaagaacaggctcatcgagactcctcagttatattatacttttaaatgtactttctcaggaaattaagccttccatgtgtgctagcagagaaagatttttattttgttttgtttttctaaaggatgttttgaaggttgctattaagtttgtggttgaaagataatgaacttaggtagccgatctgcagtcaaatataccaccactaaaatataaatatttgttcttttgcagtcagtatctgcagaacagtaagcgaaagagccggcctgtgagtgtaaaaacatttgaagatatcccgttagaagaaccagaagtaaaagtaatcccagatgtaagtacgtcttttaaaaatagtcttagaaataatacaaaggatgaaacactagctagataaatattagcctaagcattaaagttttggagcctcattagaaggctgccctcgagtgtgtgtatcatggggtcattatggagatggaactttgtttttttcataagtaaagcccttggtccaaggttcaagacagtgtagctttctgaccaatttcactaaagtgcaagtagtgtcatagtgaagacagcgatggtaacaggcattctcagctgctgatttgtaaattttctcttctccctggcctgtgtctactcataggaagcagttgcttccttttgtagcttggacaatttgtggctatgatacctttatgttcttccacaggaccttatttgatagacatgatagatgggttgagaaatcagcttaattaaatagttggtcattttatatgctcaattaactgtgccatctcattgtctcttaaaaaggacaaccagacggacagtggtatggttcttgcctcagaagagctgaaaactttggaagacagaaccaaattatctccatcttttgggtaagactcagccatattaaaaagacaaatttcaataggaatttttggaaggaacttaggactttcagtgtaagtgcagaattttccctatggggtctttgttggttggagaaattagcatcaatttaacaaataaagaatggaaactaaccacacaataaaattaagtgataaatctaaaaataatctgaaataaattagagaatttggtcaatttttatgagaattcatgaatactagggaatttctgtgtatatttactgtggtcagtaatggctaaatgaaaaaggtgattggatgtgatccgtaaagctgtcaatatgattacaatctttgtggactctgaagaatttttaagtctgtatacaaatgggtgcatctgtgcttaagaagtatgatatataaataagccaatatctatttgtttgagacatttaaatattattgtctgaattcgaagtatttcattgtgagaaaagtattaaaattagttttaaatataatctcccttctatggctcagtaggaatttgtaggtgtcttgaatacgtgtacgttctcttaacataacaaatcaatgaaaatctatatttataagaataatagaataagtgtagttatgtatttgctggagtttatttgctagagtattcttacctaaaggtaagaatagaggaggttttgatctgcttataatcttttatataaaatgggaatactcatgggtttttgaataatgctcataccaaaaagaaaacaaacaaaaaaaaccccaacatattaaaaggtgccattgtgctattttattgttttctttaaggcccaaggtaagaaattgtgaaagtcaatgatatgtttcattcattgattcaaaaaatgtttattcggcaagtatcatgtgcagagcaccatgccattgcttgagacacctacattagttttgttggggttgaattgaaagaaaaaattgtatttctcattatttgaagtaacttttaaactatgtataaacacgagttactaaaattcccttttgcagttttaacatgaagaagttggggaaaacacctattaccgggaaaaaacaccttagaatggcttgtgaaagtgtaaatcctgaagttttagatcaacacagcctgcatttctaggctttgacatgattaccgtctgtcaggattccatgccattgaaaacattttctagttgctgctgagtgacaggggttctcagtccttccaaggaatgtggttttgatgagtaaaaagcagcgtttgatatgtctggcttgactgcacacatgcttcaagttattaaagtttaaagttgctcaagagctttattacaaccatacacatgccccgtaattcccaaattgccacaataggaaaagcacaagtgaaatttaagaacatcccaatttccttgaatatcatgcaagtggccctttggcgcctgtcactgtatacaaatttgtcaatctgcgaggccataaacatgttccatcagttggggcctttgcataactcgagagaactgcctttcatctcatttgaggcttgaaagacttggacctgagtaagaggacttatctgcaactactaattcatgcgagtacctgaaaatagaccttgtccctgtaaacctgctatgctgattaacaactgggagagatacggggctgcggtctccagggagatggcagccatatggagttgggaatggggtgagggtaaaaagcaaaagaattgtcttctctctgccaactcctttgtttgccatttcttctgcagtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacacccccaactcctggacatcacatgagaggtgctgctcagattttcaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctctttttcattcatttaaaaagcatttatcatgccccctgctgcgggtctcaccatgggtttagaacaaagacgttcaagaaatggccccatcctcaaagaagtagcagtacctggggagctgacacttctgtaaaactagaagataaaccaggcaatgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttacgtgtctcctggccaagccccaggaaggaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttgaa

SEQ ID NO: 2 - RNA, 4071 nt, Homo sapiens
augcagagcaaggugcugcuggccgucgcccuguggcucugcguggagacccgggccgccucuguggguuugccuaguguuucucuugaucugcccaggcucagcauacaaaaagacauacuuacaauuaaggcuaauacaacucuucaaauuacuugcaggggacagagggacuuggacuggcuuuggcccaauaaucagaguggcagugagcaaaggguggaggugacugagugcagcgauggccucuucuguaagacacucacaauuccaaaagugaucggaaaugacacuggagccuacaagugcuucuaccgggaaacugacuuggccucggucauuuaugucuauguucaagauuacagaucuccauuuauugcuucuguuagugaccaacauggagucguguacauuacugagaacaaaaacaaaacuguggugauuccaugucucggguccauuucaaaucucaacgugucacuuugugcaagauacccagaaaagagauuuguuccugaugguaacagaauuuccugggacagcaagaagggcuuuacuauucccagcuacaugaucagcuaugcuggcauggucuucugugaagcaaaaauuaaugaugaaaguuaccagucuauuauguacauaguugucguuguaggguauaggauuuaugaugugguucugaguccgucucauggaauugaacuaucuguuggagaaaagcuugucuuaaauuguacagcaagaacugaacuaaauguggggauugacuucaacugggaauacccuucuucgaagcaucagcauaagaaacuuguaaaccgagaccuaaaaacccagucugggagugagaugaagaaauuuuugagcaccuuaacuauagaugguguaacccggagugaccaaggauuguacaccugugcagcauccagugggcugaugaccaagaagaacagcacauuugucaggguccaugaaaaaccuuuuguugcuuuuggaaguggcauggaaucucugguggaagccacggugggggagcgugucagaaucccugcgaaguaccuugguuacccacccccagaaauaaaaugguauaaaaauggaauaccccuugaguccaaucacacaauuaaagcggggcauguacugacgauuauggaagugagugaaagagacacaggaaauuacacugucauccuuaccaaucccauuucaaaggagaagcagagccauguggucucucugguuguguaugucccaccccagauuggugagaaaucucuaaucucuccuguggauuccuaccaguacggcaccacucaaacgcugacauguacggucuaugccauuccucccccgcaucacauccacugguauuggcaguuggaggaagagugcgccaacgagcccagccaagcugucucagugacaaacccauacccuugugaagaauggagaaguguggaggacuuccagggaggaaauaaaauugaaguuaauaaaaaucaauuugcucuaauugaaggaaaaaacaaaacuguaaguacccuuguuauccaagcggcaaaugugucagcuuuguacaaaugugaagcggucaacaaagucgggagaggagagagggugaucuccuuccacgugaccagggguccugaaauuacuuugcaaccugacaugcagcccacugagcaggagagcgugucuuuguggugcacugcagacagaucuacguuugagaaccucacaugguacaagcuuggcccacagccucugccaauccaugugggagaguugcccacaccuguuugcaagaacuuggauacucuuuggaaauugaaugccaccauguucucuaauagcacaaaugacauuuugaucauggagcuuaagaaugcauccuugcaggaccaaggagacuaugucugccuugcucaagacaggaagaccaagaaaagacauugcguggucaggcagcucacaguccuagagcguguggcacccacgaucacaggaaaccuggagaaucagacgacaaguauuggggaaagcaucgaagucucaugcacggcaucugggaaucccccuccacagaucaugugguuuaaagauaaugagacccuuguagaagacucaggcauuguauugaaggaugggaaccggaaccucacuauccgcagagugaggaaggaggacgaaggccucuacaccugccaggcaugcaguguucuuggcugugcaaaaguggaggcauuuuucauaauagaaggugcccaggaaaagacgaacuuggaaaucauuauucuaguaggcacggcggugauugccauguucuucuggcuacuucuugucaucauccuacggaccguuaagcgggccaauggaggggaacugaagacaggcuacuuguccaucgucauggauccagaugaacucccauuggaugaacauugugaacgacugccuuaugaugccagcaaaugggaauuccccagagaccggcugaagcuagguaagccucuuggccguggugccuuuggccaagugauugaagcagaugccuuuggaauugacaagacagcaacuugcaggacaguagcagucaaaauguugaaagaaggagcaacacacagugagcaucgagcucucaugucugaacucaagauccucauucauauuggucaccaucucaauguggucaaccuucuaggugccuguaccaagccaggagggccacucauggugauuguggaauucugcaaauuuggaaaccuguccacuuaccugaggagcaagagaaaugaauuuguccccuacaagaccaaaggggcacgauuccgucaagggaaagacuacguuggagcaaucccuguggaucugaaacggcgcuuggacagcaucaccaguagccagagcucagccagcucuggauuuguggaggagaagucccucagugauguagaagaagaggaagcuccugaagaucuguauaaggacuuccugaccuuggagcaucucaucuguuacagcuuccaaguggcuaagggcauggaguucuuggcaucgcgaaaguguauccacagggaccuggcggcacgaaauauccucuuaucggagaagaacgugguuaaaaucugugacuuuggcuuggcccgggauauuuauaaagauccagauuaugucagaaaaggagaugcucgccucccuuugaaauggauggccccagaaacaauuuuugacagaguguacacaauccagagugacgucuggucuuuugguguuuugcugugggaaauauuuuccuuaggugcuucuccauauccugggguaaagauugaugaagaauuuuguaggcgauugaaagaaggaacuagaaugagggccccugauuauacuacaccagaaauguaccagaccaugcuggacugcuggcacggggagcccagucagagacccacguuuucagaguugguggaacauuugggaaaucucuugcaagcuaaugcucagcaggauggcaaagacuacauuguucuuccgauaucagagacuuugagcauggaagaggauucuggacucucucugccuaccucaccuguuuccuguauggaggaggaggaaguaugugaccccaaauuccauuaugacaacacagcaggaaucagucaguaucugcagaacaguaagcgaaagagccggccugugaguguaaaaacauuugaagauaucccguuagaagaaccagaaguaaaaguaaucccagaugacaaccagacggacagugguaugguucuugccucagaagagcugaaaacuuuggaagacagaaccaaauuaucuccaucuuuugguggaauggugcccagcaaaagcagggagucuguggcaucugaaggcucaaaccagacaagcggcuaccaguccggauaucacuccgaugacacagacaccaccguguacuccagugaggaagcagaacuuuuaaagcugauagagauuggagugcaaaccgguagcacagcccagauucuccagccugacucggggaccacacugagcucuccuccuguuuaa

SEQ ID NO: 3 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 4 - protein, 678 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLGRETILDHCAEAVGMP

SEQ ID NO: 5 - protein, 712 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSE

SEQ ID NO: 6 - DNA, 24252 bp, Homo sapiens
gccgagccagccccttcaccaccagccggccgcgccccgggaagggaagtttgtggcggaggaggttcgtacgggaggagggggaggcgcccacgcatctggggctgactcgctctttcgcaaaacgtctgggaggagtccctggggccacaaaactgcctccttcctgaggccagaaggagagaagacgtgcagggaccccgcgcacaggagctgccctcgcgacatgggtcacccgccgctgctgccgctgctgctgctgctccacacctgcgtcccaggtaggggctgggtcccgaacgcctgcgcctggaacagggtctaattgagggggttgggggttgtcagaggatgagttggaggaatgcggttcagtcctcagcatcctccctaatcaaataatagtaattctcgtgctttgtgcaacgccacgcggcgcagtacctggcactcagtagctaaggaaatattagtggagcaaaggcatttagctttacataatttagtgagtgcttttttttttttttttttttttttgacagagtctcactctgtcgcccaggctggagtgcagtggcgcgatctcggctcactgcaacctctgccgcccgggttcaagcgattctcctgcctcagcctcctgggtagctgggattacaggcgcctgccatcgcgcccggctaatttctgtatttttagtggagaccgggtttcactctgttagccaggatggtctcaatctcctgacctcagatgatccacctgcctcggcctcccaaactgctgggattacaggcgtgagccaccgcgcccggcccaagtgttctttttttaaatggagttcttcgaagcctcttccttgcaatttcaaactaggcgatgggactttattaatttcgtttcgcagaggaaactagggcacagagaggttagataactggcctaaaatcacacagccagtgcttgaatacgcaggatctgaccctgcagcgccccccagcgctctccacgctgctgggtctccccctctgagaaacggggggacaggacccccttttacaaaaggcccaaagggaggctgactgagcggcgcagagccagtgctggagacccgggactgtccctcaggacctttccctctcactgaggcgactctcacttacttccccggaaaatgtggggggctctgggtcgaggaattcgagaaggaactgagtcagggcgggtggccacagggtgttggggccgcgatgaataacccggaaagcgctcgagaccgcgggaggccgggaatgagtaacagctccgggatactccgaacgcgcagctggaaagggatgtccgggaaggcccggaggtcggggaccgggcctagggactgggctgcaatctcggggcggagcctggggcggggagagagtgtcggggaggagccagagggcggggctggaacctcaaggaagagctacgggagaggttacagaccgaggaagagctaggagcggggctagaacctcgaggcggagccagagggcggggttataacctcgaggcgtaaccagagggcggagttataacctcgggaggagctagaaggtgggactagactcttgaggggcaggattataacctcggggaggagctagagggcggggctggaacctcaaggaggggctaggggcggggttataagcttggggaggagctacggggctgggctaggacctcaaggggccaaggggcgggggttacgacctcggggaggagctacagggtggggctgaaacctcaagaagggactggggcggagttatgacctcaggggaaaactaggggcggggagagctaaagggcgaagttagaatctcaaggaggagctagagggagggactaaagcctcgaggaggagctaggggcggggttatggcatcggggaggagctagggggcgggaagataaggtcaggaagagctaaagggcggggctagaacctcgacgatgagtctggagcggggccaaaacctagttggagagctggagggcggagacagaacttcgcgggaaactagagtatctaacaatagaaactccggagggctgatggggccgggcctagaatttgggaataaattagtgggccgggagaggctctggagccgggtagaaccagggggagtgagtggagggatagttccattagggcactgggagtgacggtataacataaagatcgacgcgggtggggcaaggctagaacgtccccagcagaactggagaggcgtaatcgaccgagggccggtgcggtgagaaagacctaataggagcaggaatagaacgtactgatagagagggcggggatacgactgtcaggatatacgcctcacgaggacagaatgaaaggaaaaacgggccaaggcaggactttgggaaaggacttgtgggcagggatagaacgttcagttagtggggtggaggtagaacgtggacaacggacagactggaagtaccgccggccggaacccagcagaacatggacacgaatctgaatggacggggcctggagacttggtgttggtattgggacatgcaggggtgagcgggggtcttggagctaagcgtagttaacctctcctctcctctccctccttcccccagcctcttggggcctgcggtgcatgcagtgtaagaccaacggggattgccgtgtggaagagtgcgccctgggacaggacctctgcaggaccacgatcgtgcgcttgtgggaaggtgagcttccccccaacccacacacccctccagcgcagcatggaacgcgagtgattgagaaaccttcctcaccagtaactgggacatgcaaactaaaaacaaacaaacaaggagagcccatttcacacccaccagattggcaagcatttaaaaatctgacaatacccgggttagtaaggatgtggagttgcagaccgtgtctgtggtgggggagtaaattggcacatcaaccacttagagcgattcggcaatatctagtccagatgaagattaacatatgctacagctcaactcctcgccatgcagaaatgccaaagaaccttggccacatatacccagggacacatgtatacgaatgttcttagcagcaatgtttgtgacatggaaaattggaaacaggctgggcactggctcacacctgtatttccagcactttgggaggcagaggcggatgaattgcttgaggtcaggagttcaagaccaggctggccaacatggtgaaacacccgtctccacacacacacaaaaaaggaaaaattagccgagtgtggtggcacgcacctgtaatctcagctacttgggaggctgagacaggagaattgcttgaacctgggaggtggaggttgcagtgagctgagatcatgctactgcactccagcctgggcgacagagcgagatcctgtcaaaaaaatgaacgaaagagagaaaggtgggccgggcatggtggctcacacctgtaatcccagcactttgggaggctgaggcaggtggattacctgaggtcaggagttcaggaccagcctggccaacatggtgaaaccctgtctctactaaaaaatacaaaaattagccgggcttggtggcaggcaccagttatcccagctactcgggaggctgagacaggagaattgtttgaacccaggaggcggaggttgcagtgagctgagattgtgccattgcactccagcctgggcgacaagagcaaaactccatctcaagaaagaaagaaagaaaggaaaggaagagagagagagagaaagagagagaggaagggagggagggacggagagaggcagggagggagggagggaggagggaggaaggaaggggagaaagaaagaaaagaagaaaatggaaacgactttatgtcagtcagcaagtcagcagcagaagagaaacactgtggtaataattacataatggagcgctctactgcagcttaaagaaggaaatagagctgcagataccaatgtggacacatctcagaaacctaaggttgagagggaaaaaaatgttgcagaatatgatacgttttttataagctttaagaacacatgaaacaacagtatatgaagccaagcacagtagctcacatttgtacccctagctactcaggaggctaaggcagaaggattccttgagcccagaagttcgaggttgtggtgagctatgattgccccagctgggacaacagagaccctgtttctaataaataaataaataaaataaagttacatttgaggcatctcagtactgagatcactaagctgagggtgtgggggagggatagatggatccagaaagtcccagttgtagtgctgtcactacaagagcaggagtgggacacagtggctcacacctgtaattccagcactttgggaggccaaggtgggaggatcacttgaggccaggagtttgagatcagcctgggcaacatagtgagatgccatctgtataaaaaatttaaaaattagcagggtgtggtgatgtaagcctgtggttccagctactcgggaggccgaagcaggactgcttgagcctgggaggtcaaggctccagtgagctatgattgcaccactccagcctgggtgacagagcaagaccctgtctcccggaaaaaaaaattaattaattaattaattgaaataaaatagagcaggctccttgttggggtgggggtggaggcaaggttaactctagaaaaagacagagttcgactcaaataacagaagaggcctagcagagatcagccctgggagtggatgaccttgagaccaggagtgtcagaggctggttgagctgagaggagcctctggggtaatgaagatcccctccttgcagaaggagaagagctggagctggtggagaaaagctgtacccactcagagaagaccaacaggaccctgagctatcggactggcttgaagatcaccagccttaccgaggttgtgtgtgggttagacttgtgcaaccagggcaactctggtgagtagggcagcccttgccatccccaaccccaagccatccccagctcaacctcatcatcatcccaatcctattcttaactttacatcagctgaaccccaactccatgccttatccaagtcccaaccccaacccaacccatctctagctcagctccatccccaactcattcttgaccccatatccaaccccaactcaacttcacctccaatcctatgcccaacttcatgttaccctaacactgttcccaactcaaacccagccctgttccaagcacagttccaaccccatccccaaatcaaccccatccttatctcagactacccaaatccccaccccaacgacacaattactttcatccttaacccaatccccatcctgcatgtttgtatgtatggttgttatgtataacttgcaactctgcccttaacaagctcacacgcatgcatgagacatagatgagatgtctcagtgatgtttgtccatctgactgggcaggagatggtcctgcccaatccattgggacgggagatggtccaagcaatagatactgaggggccaggattcaagcaaagacttgacgttcttaagggaagtcgaaagaggggaagattaaatgggccgcagtaggctgggagaatcctctgcaatagatggtctctgacttgggcagtgaaaaaacaaatgtgaggaaatgtccataataggctgggtgtggtggctcatgcctgtaatccaagcactttgggagaccaaggcgggagaaccgcttgagcccaagagtttaagaccagcctgggcaacaaccataagcccctttgtcgaatttcatggcttcaggctctgatcattttaagaaaaagacttagaaaaggtcaaattttcctaaattcccacaggagaggaagggtggggaaggaatgggtccatggtgcatggatccatccgtgaaagagattcctctttctggacctggtgaggaaggaaatagggtaaggccagtctagtcatggttggtgacatcagtgagcagagtgctgcgtttgcactgggttttctttgttcttcattcatcctgattctgcctggttttcatccatcctgacctcaaacactaatccgctggtggggccaaatcagatgtttgttttgaggaagtgacgagaggatgtgggcttcccgtctcggtgagatgtgcttaaggaaagcaccaatacttgggtctaagtagtgcattcaatcagagatgatagcgaaaatatttaacaatattggtcagtgctgggacaaactcctggagtccgctgctgggcctggtgttgcccctttccatgatgggtcacagggacatgaagggtcctggggagagagttggcagtggtcagggatccaccaggagcttagagcagcagctatcaattcaggaatctaataactgagatacctatatcatccctttttcttttcttttttctttctttttttttttgtttccttttgtttttgttttttttttttgtttgtttgttcgtttgagatggagtttcactcttcttgcccaggctggagtgcaatgccacgatctcggctctctgcaacctccgcctcctaggctcgagcgattcttctgcctcagcctcccaagtagctgggattacaggcatgcgccaccacgcccagctaattttgtatttttagtagagatgaggtttctccatgttggtcaagctgatcttgaactcctgacctcaggtgatctgcccgcctcagcctcccaaagtgctgggattacagacgtgagccaccgtgcccggcctcatccccatttctaatgccattgtaccctgcttgtggcaacttgcatagttggttaaactgcactcagtacttacatgatgctgggaacacagcagggaccaagacagccttgtgccctgcctgactccatgggactcacagtcctgtgtgtgtgtgtgtgtgtgtgtgtgtgtgtgtgtgtgtgtgtgtgtgtgtgtaggggtgacagacccatccatcaccagacagtgactgattattaggttaatgcaaaagtaattgtggcttttgccattaaaagcaatggcagaaaccacaattacttttgcaccaacctaatagatttgtctgggctatgaagggggaaacacaggcagaggggtcagggctgggatgagggaagagtaggagctgtgggaccctagaggagatacctgactcaggtcagggaaggtttcctaggggaagcaacagctaagctggatcttgaaggatgagtaggaatcagactattaaacagctaatggaggctgggtgcagtggctcacgcctgtaatcccaacactttgggaggcccaggtgggaggattgtttcagaccagcctaggcaacatgactctacaaaaaaattaaaaaattagccagatgtgatggtacatgcctgtgatcccagctccttgggaggttgaggcgggaggatcactatagcccaggtggtggaggctgcagtaagctatgatcgcaccactgtaccccagcctgggtgacagagcgagatcctgcaaaaaaaaaaaaaaacagtggaaagggttttccagcagagggagcagcagatacaaaggtggccaaaagcagtgtggctggacaaaggaaacagtacagtggggcgagatgagctggaaagcttggcaggggcatgttacacaggggtttctagacattgtcctgggagcactggggcatcacagaagattttaaacagcgaatgtcatgatcgcaattatttatttatttatttttgagatggagtttcgctctttttgcccaggctggagtgcaatggtgcgatctcagctcactgcaacctccgcctcctaggttcaagtgattctcctgcctcagcctcccaagaaactgagattacaggcacccgccaccatgcccggctaatttttttgaatttttagtagagatggggtttcaccatgttggccaggctggtctcgaactcttgacctcgtgatccacccacctcggcctcccaaagtgctgggattacagacgtgagccaccgcgcccggcccccatttatatttttaacaaattattctgacctcaggacagaagatagactgaaaggaagcaagagtagatgaggggaaaccaacttgggggtgggcgaggttgtccaggtggagaagagggcagcaggactagggtgtcaggtgtgcagaggaagaaaagtggatagatttaagagctattcaggctaggcacagtggctcacgcctgtaatcccagcactttggaaggccaaggcaggaggactgcttgatcccaggagttggaggctgcagtgatcacgccactgcactccagcctaggcggcagagcaaaaccctgtctcaaccaaagccgaacaagagctattaagaagcaggccttagtggtggattagatgtgggaggggacctgcaattctggtcatgatggagtcacttgtatcagaccaacccttctgccaatgacagttgcaaatgctggacaaataataaacaattattggaagtcactggagagcagcaaatgaaaggagaaacttggctgggcaaagtggttcatgcctgtaatcccagcactttgggaggccaagacaggtgaattgctcgagctcatgagttcaagaccagcctgggcaacatggcgaaaccccgtctctactaaaaatacaaaagttagccaggcgtggtggcacatgcctgtggtcccagctacttgggtggctgaggcacaagaattgcttgaacccgggaggtagaggttacagtgagccaagatcacacgactgcactccagcctgggcgacagcgcgagactccatttcaaaaaaaaaaaaaagaaaaaagaaagaaaggagaaacttgaggcgctacaatctctgaaataacagaagtgtagaatttgggttccacagacaacctggctttttcccggagcgcacttgccaatctgctgctgtggaagggaaatagagcccagacggaaagagagtcgctagactgagaagacggaggttggagtttggggctgccagaatgtctggaagatgagagttaaaatcctggaatggatgggaccacagaaaagggagttcccaacctgcaaacaaatctccctcaagtcattggcaagctctacatgcacagggtgagagtccaagaaacttggaagagaaaaatagcaactgggaagctacaaaagcgaacaaggcttccagcagacatgtgatgctggggacacaggttgccaagctgaagaggccttggtaagcatctcaggctttccccctaagaccaccccaggggtaagggccaaagtgaaatagaccagccctaaacaagcctaggtccagtccttgacaggaatgaaaacctgactctctgtggaagaagaaaacatcgtttaggcccttgacagatttttatctagaatgtctggcgtctgatcaaaatttacaagatagctaggcatggtggcatgcgtctgtagtcccaactacttggaaggatttcttgagcctgagagttctgggctgtagtgtgctatatcaaccgggtgtctgcactaagtttggcatcaatatggtgatctcccaggagcaggggaccaccaggttgtctaaggaggggttgaagcagcccaggtcagaaatggagcaggtcaaaactcctgtgatgatcagtagcaggattatgcctatgaatagccactgcacaccagcctggacaacatagcaagaccccatctttaaaaaaaaaaaaaaccaggccgggcatggtggctcaagcctgtaatcccagcactttgggaggccgaggtgggaggattacaaggtcaggagttcatgaccacactggccaacatggtgaaaccccttctctactaaagatacaaaaaaaaaaaaaattagccgggtatggtggcacacacctgtaataccagctacttgggagactgaggcaggagaattgcttgaaccagggaggtggaggttgcagtgagccgagatcacaccattgcactccagcctgagcgagagggcaagactctgtctcaaaacaaaacaaaacaaaacaaaacaaaacgcaagactctgtctcaaaacaaaacaaaacaaaaaacaaaaaataaaaaacagtaacaaaaaaccttacaaggtatcctgaagaaaataaaagggaccaattgaccagaaaccaagagaaaaatccaactgtagaaacagaattgtagcaactcagatattggattataggacaggaaacatgaaaattgctacaatcgatatattcaggaaaacagaagaaaggatatgttttagcacctggaatccattttttaaaagagtccaacagaaaatatagaacagtgactcaaatgaagaactcggcagttatgcttgatagaggtcagaaacagcagagcaaaaggattaaggagctagaagacagaacagtagaagcatgtcggttgatgcacagatgtggaagataagggggtctgccaggtttctgccccggggactggatggggccgccttggacgcgtggacttcaagaaaaagagcagttgtgggagaagatggggagttttgtttaggacaggtgtttgaggtatagccatggggcctcgaagagacagttgcgtgtctggagctcagagagaagtctagactagagagagatttgtgagtgatgagcagatagatggaaatgaagtcatgtcaatatttaccccatgagagagtctatactgtgagagataaaaagggcttaaaacagagccctggccaggtgcggtactcacgtctgtaatcccagcactttaggaggccgaagcggcaggatcatttgagtccaggagttcgagactagcctgggtaacatagcaagaccttgtctctacaaaaatacaggaatcagccaggtgaactggcacatgcctgtagtctcagctactcagtaggctgagatgggagaatcacctgagctcagggaggtcgaggtggcagtgagccactgcactccagccttgggacagagcaagaccctatctcaaaaaaaaaaaaaaagaaaagaaaagaaaaaggaaaatagccctaaggatcattggcatttacaagagttcacaccagagacccatgagtgaccacccagagtgggcagaactagaaatggggaattcaaggggcagtgggagctcagagcaggtacccgttccaggctgatgggtcagggagggcttcctagaggaggtgaaattgaagatgacagctgaagaatgaataggagttagagaaagtagggctcaaggaaaaggtgttctaggtatgaacagctatgttggcaatggtgagaatttgtgttttcattttcatggaagtttgtgtgagaagacgtgggtggccttgtagttatttttctttgagggggacgattgggctttggatgtaaatgattcgcctaattacagctagcggcctgccagttgagaaccttggctgacatggtcctctcacttttttaagacttggcctggactcagcttagatgtgtcttgggtcaggacggggagggcatgccaagcatgggaaatggtgtgagccaagggctgggtagaggaatgactctggtgtttccagggaaaagtaacggatccagttggccaacaccacaaggttcgtgcaaaaggcgaagtgatcaggaatcagtcaagtgagtctttgatccctgggacaattaggtagtgtattatctattgcttatctattatctattgctgcattacagattgcagcaataaaataagcatgtgttatttcacaaagtttctgaaggtcaaaaaccctggagtggcttagctaggtggttctggctcaggatccttcatgagcctgcagtcgagatgtcagctggggctgcagtcatctgaaggctcctaaggtagcttcactcacatgaatgctcccctcccatggctttggcacaggcctcagttctgccacgtgaactttcccatggtgctacttgagtgtcctcacaacatggctgctgcttgccccagaaaaagtgatccaagtgagagagccaggcatgtgttctatgatctagccacagaaggcatacaccatcatttccataatatccagttgactacatagggcatccttattcttttcttcttcttttttttttttaatttaaagacagagtcttgctatgttgcccagactagtctcaaactcccagactcaagcaatcctcccacctcggcctcccaaaatgctgggattacaggcatgagctgctgcacccagccagccctattctttaagggcatgaataccagggagcagagattattgggggccattttggagacaggctaccacagggaacaatagaagggttttaagtaattgctcattcattcattcatttaacaaatatgcattgaatatcttatctgtccctggccatgtgccaggtggtgttgggacacagtagtgatcaagacagcccctggccctgccctcctgggggctcacagtccagtagaggagacatacctgtcaccagacagtgaccacccagagtgggatgggggagtacaggggtgtgtgggagcctggtggatcaggaagggcttcctagaggaggtgaaattagagctgacacctgaagaatgagtaggagttagggaaggcagagagcaaaggaaaggtgttctagacagaaacaacggcatatgcaaaggcctggatgccagggaacatgtgtcgtcgcagactgtgggaagtcagtgtggagcagagtctcagggtgtagtggagggagacgaggcaggagaggtgctcaggccccagtatttgataggctttgaatcccatgctgaggatcttggaatttattttcagggcagtagggagctatggaagagtcttgagcagaggagagacagggtcagatatgagtattagaaatatccttctaagggcccgagtgcagtggctcatgcctgtaatcccagcactttgggaggccaaggcatgtagatcacttgaggttaggagttcgagaccagcctggccaacatggtgaaaccctgtctctactaaaaatacaaaaattagcccagcgtgatggtgcacgcctgtagtcccagctattcgggaggctgaggcaggagaatcacttgaacccggggagtggaggttgcagtgagccaagatggtgccactgcgttccagcctgggtgacggagtgagactctgtctcgggaaaaaaaaaaaaaaaaaggaaagaaatgtttctagggccatgtggagatgctgcactggggtgagtagggggtgacaagaatggaaacccagaggctagggaggtagctggagttgaaggaggtctgagctggactggtccatggggctggaaaggagggggtggattctagaaagattcagcaggcagaataggcagggctcaatgactggctgcagggtgggataaggaggaattgattttgagtgagtccttgcagagctggggctccaggactgtccccatggagtctcactcccctccccctcttccaaacaggccgggctgtcacctattcccgaagccgttacctcgaatgcatttcctgtggctcatcagacatgagctgtgagaggggccggcaccagagcctgcagtgccgcagccctgaagaacagtgcctggatgtggtgacccactggatccaggaaggtgaagaaggtgagccccaacctgctggcaactcctcctccctgctctgctcctccctaagactgcacttaacaaacaaccccaaagtaacagggatgtcagcaagggactaagttctctcatacacaagaggtccagtgataggaagtccagggccacaaggacaggtccagagtcaccaggaacccagatgccttctcatttctttttatttatttatttatttattttttgagacagagtcttgctctctgtcacccaggctggagtgcaatggcatgatcttggctggctgcaacctccgcctcccaggttcaagtgattctcctgcctcagcctcctgaatagctgggattacaggtgtgcatcaccacgccttgctaatttttgtatttttagtagagatggggtttcaccatgttggtcaggctggtctcaaactcctgaccttgtgatccacccgcctcaacctcccaaagtgctgggattacaagcgtgagccaccacgcccggccaccctcttgtttcttacacattacttccattctcaaaatgcctcatagtctaaaatggctgccagagctccgggtctcacatctaagttccaggaaggaggaagaaggaaagaagggacaaaaaagggggcactcctccttttgtaagggttctcctttctggaagtcccataaagcacttctatttatatcacattagtccgaacttcatcacatgactacctggagaggctggaatatgtagttttcgacaagatctatgactgttctagataaaatgagggtcttatgatgaagggtgaagtagagaatggttgaatgcctgctatttcgggcaaatcccatcttagtccctcattcacaatctgacatctgacctctgtcccctggcccccatagggcgtccaaaggatgaccgccacctccgtggctgtggctaccttcccggctgcccgggctccaatggtttccacaacaacgacaccttccacttcctgaaatgctgcaacaccaccaaatgcaacgagggcccaagtaaggaacgggagacacaggcaaggcctggggtcgggcaggggcatgcactcaggcagacagctgcgcagtcactctctggcaatcaagtcctctctgggcctctgtttgcttttctgaaaaatgggagtatcaggcctttttttttttttttttttttttgagacggagtctcactctgttgcccaggctggagtgcaatggcgtgatctccgctcactgtaaacttcgcctcacgagttcaagcgattctcctgcttcagcctcctgagtagctgggattacaggtgcccaccatcacccctggctaatttttctatttttagtagagatgggggtttcaccatgttggccaggctggtctcgaactcctgacctcaagtgatctgtctgcctcagcctcacaaagtgctggggtaacaggcatgagccactgcacccagccaggcctttcttcaaagaagaacttcgtaaggattcaatgatactatcagccctcgccaagacctgtgcttcctgtctcttttgtctctctcaaattttcccactcctcccctgctccactgccaccacctgggctgagccacgctgacttcttgcccaggttgttgcaatgacctcctctctggtctccctgcttccactcctgccaccctacagccaaggctcaacccagcagcgagtgattttttggggggtatgggggggacagggtctcgctctgtcacccaggctggagtgcactggcatgatcatggctcactgcagcctcaaactcctgggctcaggaattcttcccacctaagctactggagtagctgcgactacaggtgcatgccaccgtacccagctaattttgtatttttggtagagatggggtcttgctttgttgcccaggctggtcttgaactcctgggcttggcctcccaaagtgttaggattacaggcatgagccaccatgcaccaccaattttttattttttaatgtaaatcagatcatgtcaccctcctttctcaggcacttcaatgacttccccttcctcttagaataagatccaaagaccttattctgttggcctatgtaatctggcacctgtctgcctcacagtctttgtttagattcgtttttggggttttgtgttttctttagacatagggtcttgctctgccacccaggctggggtgcagtggcacaatcacggctcactgccacctcaaactcctgggctccagtgatcctcccacctcagcttcccaagtagctgggactacagatgcgcatcattgcacccagctacattttttttttttttctgagatggagtcttgcgctgttgcccaggctggagtgcagtggcgcgatcttggctcactgcaagctccgcctcccgggttcacgccattcccctgcctcagcctcccgagtggctgggactacaggcacccaccaccatgcctggctaatttttgtatttttagtagagacagggtttcactgtgttagccaggatgatctcgatctcctgacctcatgatccgcctgactcagcctcccaaagtgctgggattacaggcgtgagccaccacgcccggccgcacccagctaaatttttaaaacttttgtagagatggggtctcactatgttgccggggctggtatcaaactcccaggctcaagcaatccttctgccttgacctcccaaagtgctgggattacaggcatgagccgctgcacctggccttcatttagtttctttctttcttcctttctttctttttttagatggagtctcgctctgtcagccaggctggagggcagtggcgtgatcttggctcactgcaacctccgcctcccaggttcaagcgattctcctgcctcggcctcccaaagtgctgggattacaggcatgagccgctgtgctgacgttcatttggtttctataatcaccaaagcccatctggtctcatggcccttgcagatgaatatcctcccttagaacacatcttccccaagagttcaccctgctggcaacttctcatccattaggcctcagctttaatgtatcatcttcagggatgctttcactgtccctcccctccagtgtaatctagatccctgtctctattacccagcactgtcaacagatagaaatgttccctatctgtcctgtccaatattacagccaccactgtatgtggtcagtgagcacttgaaatgtgaactgaattttaagattcgatttaatattaatttatttaaatgtaaacaaccacatgtggccagtggctaccagattgggtagtgtagctctaaattgtaaattctggctgggcacagtggctcacacctgtaatcccagcacttttggaggcggaggcgggagggtcgcctgaggccaggagtttgagacaagcctaggcaacatagcaagactccgtctcaaaaaaaattttttagtaataataatagtaataacttgtaagttctggccaggtacagtggctcacacctgtactccgagaacattgggaggctgaggcaggcagattgcttgatcccaggagtttgagaccagcctgagtaatgtgtcaaaaccccatctctacaaaaaacagaaaaattggctaggcatggtggtgcacgcctgtagtcccagctactcaggaggctgatgtggaagaatctcttgagccggggtggtcgaggctgcagtgagctacaattgcgccactgcactccagcctgggcaacaaactgagaccctgtatcaaaaataaataaataagttgtaacttttgtgagagcaaggacagtttcatcttacatagttttacccagcacccagcacggtgcctggccagaaatgggactcatggggagttgaaagttggggtttctctcaagccctcgaaaaccatccccctccaaatgccattcagacgcctgaccacaaggtggctccatgacatctctggctcggagtggaagtcctggggaggtcattcaaggaagtggagatccaagcactaattttcttggtctctgtgtctctggtcttctcctaagtcctggagcttgaaaatctgccgcagaatggccgccagtgttacagctgcaaggggaacagcacccatggatgctcctctgaagagactttcctcattgactgccgaggccccatgaatcaatgtctggtagccaccggcactcacggtgaggccctctccgaggctgggagggaacacttattgggggtggagagttccgcaagagattgacttccagttaagctggagttaactgtggtgggtctccctgtggaaaaggtgggatttccttcaggtgggtgaatattattgtaaggaattaggacttgtccatcaggatgtggacatttttgccatggaaaagtggaattccggctgggtgcggtggctgaagcttgtactctcagcactttgggaggctgaggccggaggatctcttgaggccaggagtttgagaccagcctgggcaacatagcaagacccccatctctacaaaaagaaaaatggaattctttggtggatttctcgtgggttgtggggatttgctgaagacatcttgggctttcatcctgtggtgtcctggaaaggtctctgtattccagtggttctcaaactttttggtctcaacttcttcatgcttttaaaaattatttttggccaggtgtggtggctcacgcctgtaattgcaacaccttgggagggtgaggaggagaaccacttgaggtcaggagtatgagagcaagcctggccaccatggtgaaaccctgtctctacaaaaaattacaaaaattagctgggtgtggtggcgtgcacctataaccccagctactcagggggctgaggcacgagaatcacttgaacccaggaggaggaggttgcagtgagtggggatcaccccactgcactccagcctggatgacagagggagactttgtctcaaaaaaaaaaaaaaactctttattgtttatatcagtgatttctatagataaatgccacattaaaattaaaactgagaaccaggagtggtggctcacgcctgtaatcctagcactttgggaggccgaggtgggtagatcacctgaggtcgggagttcaagaccagcctgaccaacatggtgaaaccccatctctactaaaaatacaaattagtcgggcatggtggcacatgcctataatcccagctactcgggaggctgaggcaggagaatcacttgaacctgggaggcagaggttgcggtgagccaagattgtgccattgcactccagcctgggcaacaagagtgaaactccgtcccaaaaaataaaataaaataaaattaaattaaaattaaaactgagaaatcttttttttttttttttgaaatggagtcttgctctgtcgccaggctagagtgcagtggcatggtctcagctcactgcaacctccccctcctgggttcaagcaatcctcctgcctcagcttcctgcatagctgggattacaggcatgagccaccatgcccagcttatttttgtaattttagtagagacggggtttcaccatgttggccaggatggtctcgatctcctgacctcatgatctgcctgcctcggcctcccaaagtgctgggattacagatgtgagccaccgtgcccagccaaaactgagaaatctttaaagatttatttatgaattcatcttataacaataaaaactcattacacattaagataaataacattttaatgaaaaataagtatagtttccaaagaaaaaatgtaatgatagccaggcatggtggctcatgcctataatcccaatactttgggaggccaaggtgggcgggtcacttaagcctagggatttgagaccagcctgggcaacatagcaagatcctgtctctacagaaaatacaaaagttagctgggcatggtggtgcatgcctgtagtcccagctgcttgggaggctgaggtgggagggtcacttgagcctgggaggtggaggttgcagtgaacggagattgcaccactgcactgcggcctgggcaacagagcgagaccctgtcttaaaaaaacaaacaaagaaacaacaaaaaaaaaaccagccaggcgcggtggctcacgcctgtaataccagcactttgggaggccaaggcgggcagatcacaaggtcaggagttcgagaccagcctggccaatatggtgaaaccccgtctctactagaaatacaaaaattagctgggcatggtggtcgcgcctgtagtcccagctacttgggaggctgaggcagaagaattgcttgaacccgggaggcagaggttgcagtgagccgagatcgtgccactgcactccagcctgggtgacagagtgagactccatctcaaaaaaaaaaacaaaaaaaaagcgtaatgagaaaactggcattattttgctgtgttgcacatctctttaatgtccagcttaatagaagacagctgggtcctcatggaagcttctgctttcagtctcttgcaatatcacatgtcctggagtttttggaaaagcccattgtacacttatgagagtgaaaaagccatgtgaaatcacagatctcctgaaagggtctctggggtccctgagccacactttaagaaccgctgctcgcttcctttctttctttttttttctttccttttctttctctgtctctctttccctttctttctttctttcttccttccttcctcccttccttccttctttcccttctccttccttccttcctccctctctctctctttctttcttttctttcccttcttcccttcctcccttccttcttccctccctccctccctcccttccttcctttctttctttttatttgttttttgaaacagggtctcactctgttacccaggctggagtgcagtggcgtgatcataactcaccacagcctccatctcctgagctcaagtgatcctctcatctcagcctcccaagtagctgggactacagctgtttttttcttttttatgtttgtagaaacagagttttgctatgttgtccaggctggtctggaactcctgggctcaagctatcctctctccttggcttcccaaggtgttgagattacaggcatgagccaccacacctggccctgagaactgctgctttctctaggtggtagtgaaggtggcacccactgcaaggtggcaggtcacctactggagaattccagtcctgggcccaggagctggaagtctcactccgtcttctctttcctcagaaccgaaaaaccaaagctatatggtaagaggctgtgcaaccgcctcaatgtgccaacatgcccacctgggtgacgccttcagcatgaaccacattgatgtctcctgctgtactaaaagtggctgtaaccacccagacctggatgtccagtaccgcagtggggctgctcctcagcctggccctgcccatctcagcctcaccatcaccctgctaatgactgccagactgtggggaggcactctcctctggacctaaacctgaaatccccctctctgccctggctggatccgggggacccctttgcccttccctcggctcccagccctacagacttgctgtgtgacctcaggccagtgtgccgacctctctgggcctcagttttcccagctatgaaaacagctatctcacaaagttgtgtgaagcagaagagaaaagctggaggaaggccgtgggccaatgggagagctcttgttattattaatattgttgccgctgttgtgttgttgttattaattaatattcatattatttattttatacttacataaagattttgtaccagtggacaaggccaggtatgcccttctttgtagtgtctatctttgggaggataaatggtgggggacaccttttcacaggctcctggaatcaggggtcatgtttgaggcccctgcctaggttggaagtcctcctgggggctgcctccacatctatttctggactccaaatgctgacagagcaggagcactgccatcttgaacaaacaccaccattcttagttcctcttgattaaaaatcggctaaatccggacgtggtggcgggtgcctgtagtcccagctattcgggaggctgaggcaggagaatggcgtcaatccgggaggtggagcttgcagtgagccgagattgcaccactgcactccagcctgggtgacagagcaagactctgtctcaaaaaaaaatcagctaaatccagccccaaaacatcagcctaatggctaccatcagcataaccagaaacattccaaccctaagataaacccctctctgaccagaaacatgccaacccccagatagcctcccttccgaccagagactttccaaccccacaataaacttttcctcacatgaaaacattccgaacctagataagcacccccttccaaaactcttaaatatccttagtctgtaaaaaaaaaaaaaaaaaagggctctctaacctaactcagccaaaagcccccctcaggttggttttctctaaaataaacctgtccttcaccgtcaagccacatttcatgtttctttccttttcctttttcttttcttttcttttctttttttgagatggagtttcactcttgttgcccaggctggagtgcaatgacacaatctcagctcaccacaacctccacctcctagttcaagggattctcctgcctcagcctcccaagtagctgggattacaggcatgcaccaccacacccggctaattttgtatttttagtagagacagggtttctccatgttgttcaggctggtctcaaactcccaacctcaggtgatctgcccaccttggcctcccaaagtgctaggattacaggcatgagccaccgtgcctggcctcctctttctttaattcttacaaatgcaagcttccagatcacacactgaacactaaccttctagtcttctcctaaacttcaaaggttgcaactattttgttgttctaaggagacatccccacatttcccagcatgctttgggaaaggagacggatagaagataacctgatggccagaactccctgcatgcactgcagtgaggaataaggacaggagtgcacttcccctgatgtgctggggaccggggataaaggctgggctctatttcccgggatgcctcaggactgtcagaaaaagagacagaactaccatttcaatggcactctaggagcacaatattgaaaaattcatgttcttctcatcatttgctaaggcaacccatggagacaaaaattacattccccatcaaatcttgtggctaatgaagaaaaaataagatatcttcggctggctgtggtggttcacgcctgtaatcccagcactttgggaggccgaggtgggcagatcacttgaggcaggagtttgagaccagcctggccaatttggcaaaaccccgtctctactaaaattacaaaaattaaccgggtaaggtggtgggtgcctgtaactccagctactcggaaggctgaggcaggagaatcgcttgaacccaggaggaggaggttgtagtgagccaagatcacaccactgcatgacacagtgagactatccccaaaaaaaaaaaaaaaaaaaaaaagacatcttccatggtatttgggggcaggtgagggacaaggttcttgtttagggctttctctaatgacatatcaccacctgtcactgtagtggcttaaagaagtgaggtctgtacttctgcctcttgaatatgagtgactctgtgactgctttgaccagtagaatgtggtggaagttttgctgtgctatgttctggaaccaagttttaagaaactttcatttttcactttctgaatcttagaacgctcactctggggaagctggttgccatgtaaaagtactactgccctgagaccaccatgctgtgaggaagcccaagctactcatgtataaatgccatgtggagatagagccccagatgtttcagccatctcagcccaggcaccagacaagtgggtgaagaagccaccttggacatgtagccccagcagatgtgatatagagaagaaacaggaaacttggctatattagtttcctagggctgcctgtgataaattattacaaactttataaactaacacattgtgtgcctatatcaaaacatcatggaaggacaggcacagtggctcatgcctgtagtcctagcactttgggagggtgagaaaggaagatctcttgagctcaggagttcaagatcagcctgggcaacacagtgagacctcatctccactaaaaataaaaaaaaattggctgga

SEQ ID NO: 7 - RNA, 873 nt, Homo sapiens
augggucacccgccgcugcugccgcugcugcugcugcuccacaccugcgucccagccucuuggggccugcggugcaugcaguguaagaccaacggggauugccguguggaagagugcgcccugggacaggaccucugcaggaccacgaucgugcgcuugugggaagaaggagaagagcuggagcugguggagaaaagcuguacccacucagagaagaccaacaggacccugagcuaucggacuggcuugaagaucaccagccuuaccgagguuguguguggguuagacuugugcaaccagggcaacucuggccgggcugucaccuauucccgaagccguuaccucgaaugcauuuccuguggcucaucagacaugagcugugagaggggccggcaccagagccugcagugccgcagcccugaagaacagugccuggauguggugacccacuggauccaggaaggugaagaaguccuggagcuugaaaaucugccgcagaauggccgccaguguuacagcugcaaggggaacagcacccauggaugcuccucugaagagacuuuccucauugacugccgaggccccaugaaucaaugucugguagccaccggcacucacgaaccgaaaaaccaaagcuauaugguaagaggcugugcaaccgccucaaugugccaacaugcccaccugggugacgccuucagcaugaaccacauugaugucuccugcuguacuaaaaguggcuguaaccacccagaccuggauguccaguaccgcaguggggcugcuccucagccuggcccugcccaucucagccucaccaucacccugcuaaugacugccagacuguggggaggcacucuccucuggaccuaa

SEQ ID NO: 8 - RNA, 1008 nt, Homo sapiens
augggucacccgccgcugcugccgcugcugcugcugcuccacaccugcgucccagccucuuggggccugcggugcaugcaguguaagaccaacggggauugccguguggaagagugcgcccugggacaggaccucugcaggaccacgaucgugcgcuugugggaagaaggagaagagcuggagcugguggagaaaagcuguacccacucagagaagaccaacaggacccugagcuaucggacuggcuugaagaucaccagccuuaccgagguuguguguggguuagacuugugcaaccagggcaacucuggccgggcugucaccuauucccgaagccguuaccucgaaugcauuuccuguggcucaucagacaugagcugugagaggggccggcaccagagccugcagugccgcagcccugaagaacagugccuggauguggugacccacuggauccaggaaggugaagaagggcguccaaaggaugaccgccaccuccguggcuguggcuaccuucccggcugcccgggcuccaaugguuuccacaacaacgacaccuuccacuuccugaaaugcugcaacaccaccaaaugcaacgagggcccaauccuggagcuugaaaaucugccgcagaauggccgccaguguuacagcugcaaggggaacagcacccauggaugcuccucugaagagacuuuccucauugacugccgaggccccaugaaucaaugucugguagccaccggcacucacgaaccgaaaaaccaaagcuauaugguaagaggcugugcaaccgccucaaugugccaacaugcccaccugggugacgccuucagcaugaaccacauugaugucuccugcuguacuaaaaguggcuguaaccacccagaccuggauguccaguaccgcaguggggcugcuccucagccuggcccugcccaucucagccucaccaucacccugcuaaugacugccagacuguggggaggcacucuccucuggaccuaa

SEQ ID NO: 9 - RNA, 846 nt, Homo sapiens
augggucacccgccgcugcugccgcugcugcugcugcuccacaccugcgucccagccucuuggggccugcggugcaugcaguguaagaccaacggggauugccguguggaagagugcgcccugggacaggaccucugcaggaccacgaucgugcgcuugugggaagaaggagaagagcuggagcugguggagaaaagcuguacccacucagagaagaccaacaggacccugagcuaucggacuggcuugaagaucaccagccuuaccgagguuguguguggguuagacuugugcaaccagggcaacucuggccgggcugucaccuauucccgaagccguuaccucgaaugcauuuccuguggcucaucagacaugagcugugagaggggccggcaccagagccugcagugccgcagcccugaagaacagugccuggauguggugacccacuggauccaggaaggugaagaagggcguccaaaggaugaccgccaccuccguggcuguggcuaccuucccggcugcccgggcuccaaugguuuccacaacaacgacaccuuccacuuccugaaaugcugcaacaccaccaaaugcaacgagggcccaauccuggagcuugaaaaucugccgcagaauggccgccaguguuacagcugcaaggggaacagcacccauggaugcuccucugaagagacuuuccucauugacugccgaggccccaugaaucaaugucugguagccaccggcacucacgaacgcucacucuggggaagcugguugccauguaaaaguacuacugcccugagaccaccaugcugugaggaagcccaagcuacucauguauaa

SEQ ID NO: 10 - RNA, 861 nt, Homo sapiens
augggucacccgccgcugcugccgcugcugcugcugcuccacaccugcgucccagccucuuggggccugcggugcaugcaguguaagaccaacggggauugccguguggaagagugcgcccugggacaggaccucugcaggaccacgaucgugcgcuugugggaagaaggagaagagcuggagcugguggagaaaagcuguacccacucagagaagaccaacaggacccugagcuaucggacuggcuugaagaucaccagccuuaccgagguuguguguggguuagacuugugcaaccagggcaacucuggccgggcugucaccuauucccgaagccguuaccucgaaugcauuuccuguggcucaucagacaugagcugugagaggggccggcaccagagccugcagugccgcagcccugaagaacagugccuggauguggugacccacuggauccaggaaggugaagaagggcguccaaaggaugaccgccaccuccguggcuguggcuaccuucccggcugcccgggcuccaaugguuuccacaacaacgacaccuuccacuuccugaaaugcugcaacaccaccaaaugcaacgagggcccaaaaccgaaaaaccaaagcuauaugguaagaggcugugcaaccgccucaaugugccaacaugcccaccugggugacgccuucagcaugaaccacauugaugucuccugcuguacuaaaaguggcuguaaccacccagaccuggauguccaguaccgcaguggggcugcuccucagccuggcccugcccaucucagccucaccaucacccugcuaaugacugccagacuguggggaggcacucuccucuggaccuaa

SEQ ID NO: 11 - protein, 335 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 12 - protein, 281 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHERSLWGSWLPCKSTTALRPPCCEEAQATHV

SEQ ID NO: 13 - protein, 290 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEVLELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT